(19) United States
(12) Patent Application Publication
(10) Pub. No.: US 2026/0241068 A1
(43) Pub. Date: Aug. 20, 2026

(54) MIXED-SURFACE POLYAMIDOAMINE POLYMER VARIANTS AND METHODS OF USE THEREOF

(71) Applicant: Duke University, Durham, NC (US)

(72) Inventors: Bruce SULLENGER, Durham, NC (US); Lyra OLSON, Durham, NC (US); Rachel REMPEL, Durham, NC (US); Nicole HUNTER, Durham, NC (US)

(21) Appl. No.: 19/127,034

(22) PCT Filed: Nov. 6, 2023
(86) PCT No.: PCT/US2023/078845

§ 371 Date: May 2, 2025

Related U.S. Application Data
(60) Provisional application No. 63/422,769, filed on Nov. 4, 2022.

(51) Int. Cl. A61L 27/18; A61L 27/34

(52) U.S. Cl. CPC A61L 27/18; A61L 27/34; A61L 2400/12

(57) ABSTRACT

The present disclosure provides mixed-surface polyamidoamine (PAMAM) polymers comprising cationic surface groups and neutral surface groups. Also provided are scavenging apparatuses, microfibers, filters, and other devices comprising the mixed-surface polymers. The disclosure further provides methods of using the mixed-surface polymers for treatment, detection, and prevention of diseases and conditions characterized by high levels of inflammatory mediators.

### CROSS-REFERENCE TO RELATED APPLICATIONS

This application claims priority to U.S. Provisional Application No. 63/422,769 filed on Nov. 4, 2022, the content of which is incorporated herein by reference in its entirety.

### STATEMENT REGARDING FEDERALLY SPONSORED RESEARCH

This invention was made with government support under R01AR073935 awarded by the National Institutes of Health. The government has certain rights in the invention.

### BACKGROUND

Inflammation is a crucial part of the immune response to injury and illness, but excessive or prolonged inflammation can drive disease by inducing tissue injury or perpetuating the inflammation to a pathologic extent. Nucleic acids and other cellular molecules released from dead or injured cells contribute to inflammatory signaling through interaction with pattern-recognition receptors (PRRs) by acting as damage associated molecular patterns (DAMPs). PRRs for nucleic acids are usually on the inside of cells and include the Toll-like Receptors (TLRs). Activation of these receptors requires uptake of DNA and RNA into cells, thus making extracellular nucleic acids a target for therapy. While necessary for host defense and injury response, DAMPs and related pathogen associated molecular patterns (PAMPs) can also drive maladaptive immune responses and can lead to pathologic inflammation in allergy, malignancy, and autoimmune diseases like systemic lupus erythematosus (SLE) (Anders and Schaefer 2014, Wu, Tang et al. 2015, Gao, Xiong et al. 2017).

Dendritic polymers like polyamidoamine (PAMAM) have emerged as promising therapeutic candidates to mitigate such TLR-mediated inflammation (Tomalia, Baker et al. 1985, Lee, Sohn et al. 2011). Unlike specific TLR antagonists, such as the anti-TLR3 antibody PRV-300 and the TLR4 small molecule antagonist JKB121 (Anwar, Shah et al. 2019), these highly branched, monodispersed, cationic molecules can bind and sequester a wide range of negatively charged cellular molecules, like RNAs, DNAs, and nucleic acid-containing complexes, away from their respective TLRs and other internal nucleic acid sensors. The binding and neutralization of nucleic acid-containing DAMPs/PAMPs by these molecules has been termed scavenging. As data from our laboratory has shown, treatment with PAMAM G3, the generation 3 sized dendrimer, displays beneficial anti-inflammatory effects in many murine models of disease and injury, including cutaneous lupus and lupus nephritis (Holl, Bond et al. 2014, Holl, Shumansky et al. 2016, Naqvi, Gunaratne et al. 2018, Eteshola, Landa et al. 2021, Holl, Frazier et al. 2021, Kelly, Olson et al. 2022). Mechanistically, PAMAM G3 is able to prevent the binding of DNA to anti-DNA antibodies, thereby blocking immune complex formation (Lee, Sohn et al. 2011, Stearns, Lee et al. 2012). Immune complexes in SLE can deposit in the kidney and induce the production of pro-inflammatory cytokines such as type 1 interferon by cells of the innate immune system. Reducing the burden of immune complexes can thereby help slow the progression of lupus nephritis (Holl, Shumansky et al. 2016).

Despite these promising findings in cell culture and in vivo models, therapeutic doses of PAMAM G3 can cause local and systemic toxicity in mice, limiting the nucleic acid scavenger's translational potential (Kelly, Olson et al. 2022). This toxicity increases with polymer generation due to the increased density of positive amine surface charges which double with each generation (Roberts, Bhalgat et al. 1996, Malik, Wiwattanapatapee et al. 2000). In contrast, the anionic and neutral surface group variants of PAMAM with carboxyl and hydroxyl surface end groups respectively are tolerated with minimal toxicity at much higher doses (Heiden, Dengler et al. 2007, Chauhan, Diwan et al. 2009, Tang, Han et al. 2015). However, these molecules lack the charge profile to effectively bind and scavenge nucleic acid DAMPs. Therefore, improved PAMAM dendritic polymers having scavenging efficacy and reduced toxicity are needed.

### SUMMARY

Mixed-surface polyamidoamine (PAMAM) polymers and methods of using the same are provided herein. In an aspect, provided herein is a mixed-surface PAMAM polymer comprising a plurality of surface groups, wherein the surface groups comprise cationic surface groups and neutral surface groups, wherein between about 30% and about 70% of the surface groups are neutral. In embodiments, the surface groups consist of cationic surface groups and neutral surface groups, wherein between about 30 and about 70% of the surface groups are neutral.

In embodiments, the PAMAM polymer is a G3 polymer or a G4 polymer. In embodiments, about 50% of the surface groups are neutral. In embodiments, the neutral surface groups comprise hydroxyl groups.

In another aspect, provided herein is a scavenging apparatus comprising a plurality of the mixed-surface PAMAM polymer disclosed herein, and a substrate, wherein the plurality of PAMAM polymers are immobilized on the substrate. In embodiments, the plurality of PAMAM polymers is covalently bound to the substrate. In embodiments, the PAMAM polymer further comprises a crosslinker. In embodiments, the crosslinker links the PAMAM polymer to a binding label or a detectable label. In embodiments, the crosslinker links the PAMAM polymer to the substrate. In embodiments, the substrate is a glass, a silicon, a silicone polymer, a metal, a plastic, a magnetic substrate, or an electrospun fiber. In embodiments, the substrate is a microfiber, wherein the microfiber comprises a high-aspect-ratio polymeric core, the polymeric core comprising a blend of a first core polymer and a second core polymer, and the PAMAM polymer immobilized on the surface of the polymeric core. In embodiments, the polymeric core has a mean diameter of greater than 2 microns. In embodiments, the first core polymer is a copolymer comprising residues of a first monomer and a second monomer, wherein the second core polymer comprises residues of a first monomer, and wherein the PAMAM polymer is immobilized preferentially to the residues of the second monomer of the first core polymer. In embodiments, the first core polymer comprises poly(styrene-alt-maleic anhydride). In embodiments, the second core polymer comprises polystyrene. In embodiments, the PAMAM polymer is immobilized by chemisorption or physiosorption.

In another aspect, provided herein is a method of treating cancer in a subject comprising administering a therapeutically effective amount of the PAMAM polymer or the scavenging apparatus disclosed herein the subject.

In another aspect, provided herein is a method of treating cancer in a subject comprising contacting a bodily fluid from a subject with the PAMAM polymer or the scavenging apparatus disclosed herein, wherein the contacting removes invasion-promoting or metastatic molecules or particles from the bodily fluid of the subject.

In embodiments, the cancer is selected from the group consisting of pancreatic, ovarian, colon, liver, brain, breast, and lung cancer.

In another aspect, provided herein is a method for reducing inflammatory mediators in a bodily fluid of a subject, the method comprising: contacting the bodily fluid with the PAMAM polymer or the scavenging apparatus disclosed herein, wherein the contacting step reduces the concentration of the inflammatory mediators in the bodily fluid. In embodiments, the subject suffers from a condition associated with an abnormally high concentration of inflammatory mediators. In embodiments, the condition is an autoimmune disease, an infectious disease, or an inflammatory effect of radiation therapy or exposure to radiation. In embodiments, the contacting step is performed within the subject. In embodiments, the contacting step is performed outside the subject.

In embodiments, the method further comprises obtaining the bodily fluid from the subject before the contacting step. In embodiments, the method further comprises returning the bodily fluid to the subject. In embodiments, the contacting step is performed during dialysis or in conjunction with extracorporeal membrane oxygenation. In embodiments, the bodily fluid is blood, lymph, plasma, serum, urine or cerebral spinal fluid. In embodiments, the PAMAM polymer or scavenging apparatus is administered to the subject via a route selected from the group consisting of oral, topical, intranasal, intraperitoneal, parenteral, intravenous, intramuscular, subcutaneous, intrathecal, transcutaneous, nasopharyngeal, intratumoral, and transmucosal. In embodiments, the method further comprises administering a therapeutic agent or radiation to the subject. In embodiments, the therapeutic agent or radiation is administered before, concurrently with, or after the administration of the PAMAM polymer or the scavenging apparatus or the contacting the bodily fluid with the PAMAM polymer or the scavenging apparatus.

In another aspect, provided herein is a method of detecting cancer comprising obtaining a sample from a subject; contacting the sample with the PAMAM polymer or the scavenging apparatus disclosed herein; and determining a level of circulating free DNA (cfDNA), cfRNA, inorganic polyphosphates, exosomes or nucleosomes in the sample, wherein an increase in the level of cfDNA, cfRNA, inorganic polyphosphates, exosomes or nucleosomes in the subject as compared to the level in a control subject without cancer is indicative of cancer in the subject.

In another aspect, provided herein is a method of detecting metastasis of a cancer comprising obtaining more than one sample from a subject diagnosed with cancer over a period of time; contacting the sample with the PAMAM polymer or the scavenging apparatus disclosed herein when the sample is obtained; determining a level of circulating free DNA (cfDNA), cfRNA, inorganic polyphosphates, exosomes or nucleosomes in each of the samples; and comparing the levels of cfDNA, cfRNA, inorganic polyphosphates, exosomes or nucleosomes in the samples from the subject over time, wherein increasing cfDNA, cfRNA, inorganic polyphosphates, exosomes or nucleosomes over time is indicative of metastasis or advancing cancer.

In embodiments, the subject is administered a PAMAM polymer prior to obtaining the sample. In embodiments, the method further comprises administering a PAMAM polymer to the subject if the cfDNA or nucleosomes are increased.

In another aspect, provided herein is a filter, the filter comprising the PAMAM polymer or the scavenging apparatus disclosed herein, wherein the microfiber forms a porous mesh. In embodiments, the filter is configured to bind or sequester a nucleic acid, a protein, a biomolecular complex, an exosome, or a microparticle from a solution or a sample. In embodiments, the filter is configured to allow for diffusion or flow of the sample through the porous mesh.

In another aspect, provided herein is an extracorporeal filtration apparatus for sequestering a nucleic acid, a protein, a polysaccharide, a glycosaminoglycan, a biomolecular complex, an exosome, or a microparticle from a subject in need of a treatment for a disease or a condition or for the prevention of the onset of a disease or condition, the filtration apparatus comprising a first end of a blood line configured to interface with a first blood vessel of the subject for the removal of blood from the subject; a second end of the blood line configured to interface with a second blood vessel of the subject for the return of blood to the subject; a blood pump positioned along the blood line between the first end and the second end configured for the extracorporeal circulation of blood from the first end to the second end; and the filter disclosed herein, wherein the microfiber is formed into a porous mesh positioned along the blood line between the first end and the second end, wherein the microfiber is configured to bind or sequester the nucleic acid, the protein, the polysaccharide, the glycosaminoglycan, the biomolecular complex, the exosome, or the microparticle from the subject.

In another aspect, provided herein is an ex vivo perfusion or filtration machine, the machine comprising: a housing configured to host a tissue, a graft, or an organ; a first end of a line configured to interface with the tissue, the graft, or the organ and allow for the introduction of a perfusate to the tissue, the graft, or the organ; a second end of the line configured to interface with the tissue, the graft, or the organ and allow for the removal of the perfusate from the tissue, the graft, or the organ; a pump positioned along the line between the first end and the second end configured for the circulation of the perfusate from the second end to the first end; and the filter of disclosed herein, wherein the microfiber is formed into a porous mesh positioned along the line between the first end and the second end, wherein the microfiber is configured to bind or sequester a nucleic acid, a protein, a polysaccharide, a glycosaminoglycan, a biomolecular complex, a exosome, or a microparticle in the perfusate.

In another aspect, provided herein is a method of sequestering a nucleic acid, a protein, a biomolecular complex, an exosome, or a microparticle from a solution or a sample, the method comprising contacting the solution or the sample comprising the nucleic acid, the protein, the biomolecular complex, the exosome, or the microparticle with the scavenging apparatus of any or the filter disclosed herein, the extracorporeal filtration apparatus disclosed herein, or the machine disclosed herein.

In another aspect, provided herein is a method of treating a subject suffering from a disease or condition, the method comprising administering a therapeutically effective amount of a solute-cleared fluid to the subject, wherein the solute-cleared fluid is prepared from a bodily fluid, and wherein the solute-cleared fluid is prepared by contacting the bodily fluid with the scavenging apparatus, the filter, the extracorporeal filtration apparatus, or the machine disclosed herein. In embodiments, the bodily fluid is blood, lymph, plasma, serum, cerebral spinal fluid, urine or any other bodily fluid. In embodiments, the bodily fluid is removed from the subject. In embodiments, the disease or the condition is selected from the group consisting of an organ transplant donor, an organ transplant recipient, thrombosis, sepsis, infectious disease, inflammatory disease, autoimmune disease, cardiovascular disease, cancer and trauma.

In another aspect, provided herein is a method for the prevention of a disease or a condition in a subject, the method comprising administering an effective amount of a solute-cleared fluid to the subject following organ transplantation, wherein the solute-cleared fluid is prepared from a bodily fluid, and wherein the solute-cleared fluid is prepared by contacting the bodily fluid with the scavenging apparatus, the filter, or the extracorporeal filtration apparatus disclosed herein. In embodiments, the bodily fluid is blood, lymph, plasma, serum, cerebral spinal fluid, urine or any other bodily fluid. In embodiments, the disease or the condition is selected from the group consisting of an organ transplant donor, an organ transplant recipient, thrombosis, sepsis, infectious disease, inflammatory disease, autoimmune disease, cardiovascular disease, cancer and trauma. In embodiments, the subject is an organ recipient. In embodiments, the disease or the condition is thrombosis.

In another aspect, provided herein is a PAMAM composition comprising a plurality of nanofibers, the nanofibers comprising a plurality of mixed-surface polyamidoamine (PAMAM) polymers, wherein the nanofibers are less than 2 μm in diameter, and wherein each of the PAMAM polymers comprises cationic surface groups and neutral surface groups, wherein between about 30% and about 70% of the surface groups are neutral. In embodiments, each nanofiber is between 0.1 μm and 2 μm in diameter. In embodiments, the plurality of PAMAM polymers are G3 polymers or G4 polymers. In embodiments, the neutral surface groups of the PAMAM polymers comprise hydroxyl groups. In embodiments, the nanofibers form a mesh.

In another aspect, provided herein is a method of using the composition disclosed herein, comprising applying the composition to a site of inflammation or infection, wherein the PAMAM nanofibers reduce the inflammation or infection at the site.

In another aspect, provided herein is a method of using the composition disclosed herein, comprising contacting the PAMAM nanofibers with a solution comprising an anion, wherein the anion is adsorbed onto the PAMAM nanofibers, wherein the anion comprises a biofilm or microbes capable of forming a biofilm; and applying the composition in an amount effective to inhibit formation of a biofilm or prevent infectious wound formation. In embodiments, the solution is blood, serum, a biologic fluid, water, synovial fluid, or cell culture media. In embodiments, the anion is selected from a nucleic acid, heparin, enoxaparin.

In another aspect, provided herein is method of using the composition disclosed herein, comprising administering the PAMAM nanofibers to a subject in an amount effective to inhibit formation of a biofilm or inhibit growth of a microbe capable of forming a biofilm or infectious wound. In embodiments, the composition is incorporated into a medical device, filter, bandage, or dressing. In embodiments, the composition is in a medical device or as part of a graft. In embodiments, the subject has an inflammatory disease or condition, an autoimmune disease, a chronic wound or has been treated with an anticoagulant.

### BRIEF DESCRIPTION OF THE DRAWINGS

FIGS. 1A-1D All PAMAM variants prevent activation of TLR3 (FIG. 1A) and TLR9 (FIG. 1B) by effectively scavenging the nucleic acid agonists polyI:C and CpG, but do not effectively scavenge LPS, the canonical agonist of TLR4 (FIG. 1C). Each point is the mean of three replicates. Calculated IC50 values for inhibition of TLR3 and TLR9 signaling by the polymers (FIG. 1D).

FIGS. 2A-2B Mixed surface PAMAM variants capture double stranded ctDNA (calf thymus DNA) at least as, if not more, effectively than the fully cationic PAMAM G3 molecule in “sandwich-type” ELISA assays. FIG. 2A shows a plot of binding of ctDNA with data shown as mean±standard error of the mean. FIG. 2B shows a two-way ANOVA with post hoc analysis via Tukey's multiple comparison test demonstrates that the mixed polymers bind ctDNA more efficiently than G3 at 1 ng/ml and 3 ng/mL ctDNA concentrations.

FIGS. 3A-3D PAMAM G3 and mixed surface variants G3 50:50 and G4 50:50 inhibit anti-DNA antibody binding to DNA in competition ELISAs. FIG. 3A shows blocking of antibody binding. FIG. 3B shows competition with antibody binding. FIG. 3C shows displacement of antibody binding. For FIGS. 3A, 3B, and 3C, data are mean±standard error of the mean. FIG. 3D shows calculated IC50 values for inhibition of antibody binding by the polymers.

FIGS. 4A-4B When assessed after 24-hour treatment of HEK293 cells, mixed surface PAMAM variants have reduced toxicity compared to fully cationic PAMAMs. FIG. 4A shows a plot of percent viability with data shown as mean with±standard error of the mean. FIG. 4B shows a two-way mixed ANOVA with post hoc analysis via Tukey's multiple comparison test demonstrates that the mixed polymers are significantly less toxic than their same size fully cationic polymers at several matched concentrations. Selected comparisons shown.

FIGS. 5A-5C The mixed surface PAMAM variant G3 50:50 has reduced toxicity compared to fully cationic G3 when mouse macrophage RAW 264.7 cells are treated for 24 hours (FIG. 5A), 48 hours (FIG. 5B), and 72 hours (FIG. 5C). Data in graphs are mean±standard error of the mean. Below each graph are results from a two-way mixed ANOVA with post hoc analysis via Sidak's multiple comparison test identifying the concentrations at which there is a significant difference between G3 and G3 50:50 treatments.

FIGS. 6A-6C Mixed surface scavengers are associated with reduced weight loss compared to treatment with fully cationic scavengers. Scavengers were administered to C57BL/6J mice by intraperitoneal injection on days 0, 3, 6, and 9. Mixed-effects analysis of normalized body weights followed by post hoc analysis via Dunnett's multiple comparisons test identified fewer significant differences at the 20 mg/kg dosing than at the 40 mg/kg dosing. Data in graphs are mean±standard error of the mean. FIG. 6A shows body weight of mice dosed at 20 mg/kg. FIG. 6B shows body weight of mice dosed at 40 mg/kg. Mice treated with G4 at 40 mg/kg were sacrificed 24 hours after first dose due to drug toxicity. FIG. 6C shows a table comparing each scavenger treatment (dosed at 40 mg/kg) to saline treatment at day 10.

FIGS. 7A-7B Serum liver and kidney function tests indicate liver and kidney injury when treating with fully cationic PAMAM polymers. Scavengers were administered to C57BL/6J mice by intraperitoneal injection on days 0, 3, 6, and 9, with sacrifice on day 10 with blood collection for serum. FIG. 7A shows elevated AST (aspartate aminotransferase) and ALT (alanine transaminase) suggest liver injury at 40 mg/kg dose of PAMAM G4. FIG. 7B shows elevated BUN (blood urea nitrogen) and creatinine indicate kidney injury with the fully cationic PAMAMs at 40 mg/kg. No such injury is seen after treatment with the mixed surface scavenger variants. Ordinary one-way ANOVAs followed by Tukey's multiple comparisons tests were run for each of the markers; comparisons between generation matched fully cationic and mixed surface scavengers are specified. Significance: **** represents P<0.0001.

FIGS. 8A-8C The acute inflammatory response to treatment with fully cationic PAMAMs at 40 mg/kg is demonstrated by decreased lymphocyte percentage (FIG. 8A) and increased granulocyte percentage (FIG. 8B) in blood, and elevation of the inflammatory cytokine IP-10 (FIG. 8C) in serum. No such response is seen with mixed surface scavenger treatments. Scavengers were administered to C57BL/6J mice by intraperitoneal injection on days 0, 3, 6, and 9, with sacrifice on day 10 with blood collection for complete blood counts and for serum. Ordinary one-way ANOVAs followed by Tukey's multiple comparisons tests were run for each of the markers; for FIGS. 8A and B, comparisons between generation matched fully cationic and mixed surface scavengers, and between G2 40 mg/kg to saline, are specified. Significance: ** represents P=0.0017, *** represents P=0.0009 and ** represents P≤0.0001.

FIGS. 9A-9E Short term treatment with fully cationic PAMAM G3 at 40 mg/kg leads to persistent liver inflammation that is substantially less upon treatment with the mixed surface scavengers. FIG. 9A shows histological scoring for liver inflammation in C57BL/6 mice upon treatment with four doses of indicated polymer at 40 mg/kg and sacrifice at day 10 or at day 24 after 14 days of recovery. Treatments were compared using the Kruskal-Wallis test followed by post hoc analysis via Dunn's multiple comparisons test. Significance: ** represents P=0.0032 and **** represents P≤0.0001. FIG. 9B shows liver of saline-treated mouse. FIG. 9C shows liver of G3-treated mouse (40 mg/kg) with capsular inflammation (arrow). FIG. 9D shows liver of G3 50:50-treated mouse (40 mg/kg). FIG. 9E shows liver of G4 50:50-treated mouse (40 mg/kg). Bar indicates 50 μM.

FIGS. 10A-10E Short term treatment with fully cationic PAMAM G3 at 40 mg/kg leads to persistent inflammation of the diaphragm surface and muscle that is less severe upon treatment with the mixed surface scavengers. FIG. 10A shows histological scoring for inflammation of the diaphragm in C57BL/6 mice after treatment with four doses of indicated scavenger at 40 mg/kg, with sacrifice at day 10 or at day 24 after 14 days of recovery. Treatments were compared using the Kruskal-Wallis test followed by post hoc analysis via Dunn's multiple comparisons test. Significance: ** represents P=0.0066, *** represents P=0.0004. FIG. 10B shows diaphragm of saline-treated mouse. FIG. 10C shows diaphragm of G3-treated mouse (40 mg/kg). FIG. 10D shows diaphragm of G3 50:50-treated mouse (40 mg/kg). FIG. 10E shows diaphragm of G4 50:50-treated mouse (40 mg/kg). Bar indicates 100 μM.

FIGS. 11A-11B Protein droplets and nephropathic changes are evident in the kidneys of mice treated short term at 40 mg/kg with cationic and with mixed surface PAMAM scavengers. FIG. 11A shows protein droplet scores across treatment groups. FIG. 11B shows nephropathy scores across treatment groups. Treatments were compared using the Kruskal-Wallis test followed by post hoc analysis via the Dunn's multiple comparisons test. Significance: * represents P=0.0277, *** represents P=0.00030, and * represents P≤0.0001.

FIGS. 12A-12D Photomicrographs of eosinophilic protein droplets and tubular nephropathic changes in the renal cortices of mice treated short term at 40 mg/kg with cationic and with mixed surface PAMAM scavengers. FIG. 12A shows normal renal proximal tubules in saline-treated mouse. FIG. 12B shows kidney of G3-treated mouse. FIG. 12C shows kidney of G3 50:50-treated mouse. FIG. 12D shows kidney of G4 50:50-treated mouse. Bar indicates 100 μM.

FIGS. 13A-13D Photomicrographs of eosinophilic protein droplets and tubular nephropathic changes in the renal cortices of mice treated short term at 40 mg/kg with cationic and with mixed surface PAMAM scavengers. FIG. 13A shows high magnification image of normal renal proximal tubules in saline-treated mouse. FIG. 13B shows high magnification image of kidney of G3-treated mouse. FIG. 13C shows high magnification image of kidney of G3 50:50-treated mouse. FIG. 13D shows high magnification image of kidney of G4 50:50-treated mouse. Arrows identify eosinophilic protein droplets. Bar indicates 100 μM.

FIGS. 14A-14B Treatment with G3 and the mixed surface scavenger G3 50:50 reduce glomerular nephritis in lupus-prone MRL-1pr mice compared to saline or G4 50:50 treatments. FIG. 14A: female mice and FIG. 14B: male mice. For each sex the Kruskal-Wallis nonparametric test was applied followed by post hoc evaluation via Dunn's multiple comparisons test with each polymer treatment compared to the saline control. Significance: for female mice * represents P=0.0441, ** represents P=0.0014; for male mice * represents P=0.0376 and ** represents P=0.0100.

FIGS. 15A-15D Photomicrographs showing reduction of glomerulonephritis in lupus prone MRL-1pr mice after treatment with G3 and G3 50:50. FIG. 15A shows kidney of female saline-treated mouse. FIG. 15B shows kidney of female G3-treated mouse. FIG. 15C shows kidney of female G3 50:50-treated mouse. FIG. 15D shows kidney of female G4 50:50-treated mouse. Arrows denote glomeruli. Bar indicates 100 μM.

FIGS. 16A-16C Structures of the fully cationic polymer PAMAM G3 and mixed surface variants in which half available protonable amines (—NH2) have been replaced by neutrally charged hydroxyl groups (—OH). G3 (FIG. 16A) and G3 50:50 (FIG. 16B) are of the same generation, while G4 50:50 (FIG. 16C) is one generation larger. The modified structures are based on images from Dendritech Inc.

FIGS. 17A-17C ITC thermograms for PAMAM G3 and the mixed surface variants are similar. PAMAM G3 (FIG. 17A), PAMAM G3 50:50 (FIG. 17B), and PAMAM G4 50:50 (FIG. 17C). The top window for each shows the raw isothermal calorimetry data for the injection of the PAMAM polymer into a solution of CpG 1668 oligonucleotide. The bottom window for each shows the dilution-corrected and integrated heat data (dots) with a nonlinear regression fit to a two-site binding model (line).

FIG. 18 Cessation of treatment with fully cationic PAMAMs is associated with recovery of body weight. Scavengers were administered to C57BL/6J mice by intraperitoneal injection on days 0, 3, 6, and 9, with either sacrifice at day 10 or recovered for 14 additional days with sacrifice at day 24 (n=5 for each group). Data in graphs are mean±standard error of the mean.

FIG. 19 PAMAM G3, when dosed at either 20 mg/kg or 40 mg/kg, leads to liver capsule inflammation, while the mixed surface polymers elicit little to no inflammation at either dose. A multiple Mann-Whitney test with a false discovery rate set to 1% indicates that liver inflammation scores are not significantly different at 20 mg/kg versus 40 mg/kg polymer dosages. q values (false discovery rate adjusted P values) are indicated above the pairwise comparisons and are not significant. Group numbers: G3 20 mg/kg n=8, G3 40 mg/kg n=11, G3 50:50 20 mg/kg n=4, G3 50:50 40 mg/kg n=13, G4 50:50 20 mg/kg n=4, and G4 50:50 40 mg/kg n=9.

FIGS. 20A-20B Lower treatment dose of all the scavengers is associated with reduced protein droplet deposition (FIG. 20A) and reduced nephropathy (FIG. 20B). For dose comparisons of droplet aggregates and nephropathy, multiple Mann-Whitney tests with the false discovery rate set to 1% were run. q values (false discovery rate adjusted P values) are indicated above the pairwise comparisons and are all significant. Group numbers: G3 20 mg/kg n=8, G3 40 mg/kg n=11, G3 50:50 20 mg/kg n=4, G3 50:50 40 mg/kg n=13, G4 50:50 20 mg/kg n=4, and G4 50:50 40 mg/kg n=9. For both droplets and nephropathy, all saline-treated mice were scored as 0.

FIGS. 21A-21B Discontinuation of 40 mg/kg scavenger treatments trends with reduced protein droplet deposition (FIG. 21A) and reduced nephropathy (FIG. 21B). For comparing acute and recovery scores of droplet aggregates and nephropathy, multiple Mann-Whitney tests with the false discovery rate set to 1% were run. q values (false discovery rate adjusted P values) are indicated above the pairwise comparisons. For protein droplet deposition, discontinuation of G3 50:50 and G4 50:50 led to statistically significant reduction of droplet scores. For nephropathy, the multiple Mann-Whitney tests did not indicate significance. Group numbers: G3 acute n=11, G3 recovery n=5, G3 50:50 acute n=13, G3 50:50 recovery n=5, G4 50:50 n=9, and G4 50:50 recovery n=5. For both droplets and nephropathy, all saline-treated mice were scored as 0.

FIG. 22 shows the degree of activation of TLR receptors by canonic TLR agonists in the presence or absence of polymers. Positive control: “positive agonist only,” Negative control: “media only,” Polymer control with no TLR agonist: “20 PAMAM only.” Experimental conditions positive agonist+100 ug/mL->0.1 ug/mL polymer. Within each condition, L most=read 45 minutes after start of QuantiBlue readout assay, R most=3 hours after start of QuantiBlue readout assay.

FIGS. 23A-23B HEK293 cells with overexpression of TLR9 receptor were activated in the presence of canonic TLR9 agonist CpG in the presence of increasing concentrations of PAMAM G3 (32 amines), G3 50:50 (16 amines, 16 hydroxyls), G4 50:50 (32 amines, 32 hydroxyls), and G4 33:67 (21 amines, 43 hydroxyls). Intensity of TLR activation and cellular toxicity were assessed. FIG. 23A shows PAMAM-G4 with mixed surface groups (G4 50:50 and G4 33:67) outperform fully cationic PAMAM-G3 in scavenging assays (compare blue and green lines (mixed surface) to black line (pure cationic)) despite having the same or fewer cationic surface charges per molecule. FIG. 23B shows PAMAM with mixed surface groups are less toxic in vitro than PAMAM-G3 (compare black line (pure cationic G3) to all colored lines (mixed surface)).

### DETAILED DESCRIPTION

Nucleic acid-binding polymers can have anti-inflammatory properties and beneficial effects in animal models of infection, trauma, cancer, and autoimmunity. PAMAM G3, a polyamidoamine dendrimer, is fully cationic bearing 32 protonable surface amines. However, while PAMAM G3 treatment leads to improved outcomes for mice infected with influenza, at risk of cancer metastasis, or genetically prone to lupus, its administration can lead to serosal inflammation and elevation of biomarkers of liver and kidney damage. Therefore, variants with reduced density of cationic charge through the interspersal of hydroxyl groups were developed and evaluated as potentially better tolerated alternatives.

### Mixed-Surface Polymers

Provided herein is a mixed-surface polyamidoamine (PAMAM) polymer, also referred to as a PAMAM polymer or a mixed-surface polymer. The PAMAM polymer comprises a mixture of cationic surface groups and neutral surface groups. In preferred embodiments, the surface groups consist of cationic and neutral surface groups. The neutral surface groups may comprise between about 30% and about 70% of the surface groups, and any percentage or range in between (e.g. 35%, 40%, 45%, 50%, 55%, 60%, 65%, 70%, etc.). For example, the neutral surface groups may comprise between about 30% and about 50%, between about 40% and about 60%, between about 45% and about 55%, etc. of the surface groups. In preferred embodiments, the neutral surface groups comprise hydroxyl groups. However, the polymer may include other neutral surface groups including, without limitation amino acids with uncharged sidechains (i.e. serine, threonine), polymers (i.e. polyethylene glycol), and other end groups uncharged at physiological pH (mercapto-thiols, aromatics).

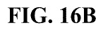

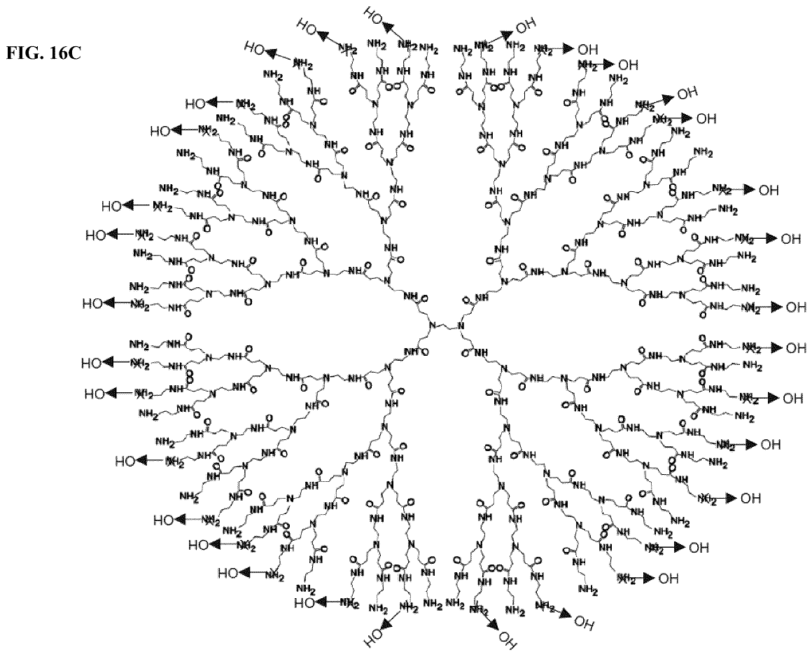

The mixed-surface PAMAM polymer may be a dendrimer or a dendron. Dendrimers and dendrons may be characterized by the generation number Gn. The generation number details the number of successive additions of the polymers base monomer. The generation number (Gn) may characterize the dendrimer's properties depending on the choice of the polymer. Properties characterizable by knowledge of the generation number and the polymer include, without limitation, the number of branch points, the size of the dendrimer, the electronic charge, and terminal moieties. In certain embodiments, the dendrimer is a G2 dendrimer, a G3 dendrimer, a G4 dendrimer, a G5 dendrimer, G6 dendrimer, or any Gn suitable for use as a scavenger. In preferred embodiments, the PAMAM polymers comprise G3 dendrimers or G4 dendrimers, and are therefore referred to as G3 polymers and G4 polymers. Preferred mixed-surface polymers are illustrated in FIG. 16.

The surface groups of the mixed-surface polymers may be any surface groups that allow for the binding of a variety of molecules and/or supramolecular assemblies, including nucleic acids, proteins, polysaccharide, glycosaminoglycan, biomolecular complexes, exosome, and/or microparticles. The polymers bind to a wide array of different nucleic acids including ssRNA, ssDNA, dsRNA and dsDNA, which may be presented in a complex with protein such as viral proteins, histones, HMGB1, anti-nuclear antibody, RNA-sensing pattern recognition receptors (e.g. MDAS, RIG-I, TLR3, TLR7, TLR8, PKR) and DNA-sensing pattern recognition receptors (e.g., TLR9, AIM2 and STING). The PAMAM polymer also binds DAMPs (damage associated molecular pattern) and PAMPS (pathogen-associated molecular pattern) as well as other inflammatory mediators. Under certain conditions, the surface groups may assist to effectively bind the nucleic acids irreversibly. Under certain under condition, the surface groups may assist to effectively bind the nucleic acids reversibly. Mixed-surface polymers may include biocompatible polymers (that is, polymers that do not cause significant undesired physiological reactions) that can be either biodegradable or non-biodegradable polymers or blends or copolymers thereof.

### Scavenging Apparatus

Provided herein is a scavenging apparatus comprising a plurality of the mixed-surface polyamidoamine (PAMAM) polymers immobilized on a substrate. In embodiments, the mixed-surface polymer includes a crosslinker. The crosslinker may link the polymer to a binding label or a detectable label. In embodiments, the substrate comprises a binding moiety and the detectable label binds with the binding moiety to immobilize the PAMAM polymer on the substrate. The binding moiety may be avidin, an antibody, or any other binding protein. When avidin is used as a binding moiety, the detectable label is an avidin-binding label. In particular embodiments when avidin is used as a binding moiety, the detectable label is biotin. When an antibody is used as a binding moiety, the detectable label is an antibody-binding label. In particular embodiments when an antibody is used as a binding moiety, the detectable label may be an antigen.

In an embodiment, the plurality of PAMAM polymers is covalently bound to the substrate. The polymers comprise a dendrimer, the dendrimer comprising a focal point, a plurality of termini, and a branched polymer between the focal point and the plurality of termini, and a crosslinker, wherein the crosslinker links the substrate and the focal point of the dendrimer. In an embodiment, the dendrimer further comprises a first crosslinkable moiety, the substrate comprises a second crosslinkable moiety, the second crosslinkable moiety capable of crosslinking with the first crosslinkable moiety; and the crosslinker is prepared by contacting the first crosslinkable moiety with the second crosslinkable moiety. In certain embodiments, the first crosslinkable moiety or the second crosslinkable moiety comprises a member selected from the group consisting of sulfhydryl, carbonyl, carboxyl, amine maleimide, haloacetyl, pyridyl disulfide, thiosulfonate, vinylsulfone, hydrazide, alkoxyamine, carbodiimide, isothiocyanates, isocyanates, acyl azides, N-Hydroxysuccinimide ester, sulfonyl chloride, glyoxal, epoxide, oxirane, carbonate, aryl halide, imidoester, carbodiimide, anhydride, or fluorophenyl ester.

The substrate may be any substrate suitable for binding the mixed-surface polymer. The substrate may be a glass, silicon, a silicon polymer, a metal, a plastic, magnetic, or an electrospun fiber. Glasses may include silica, a borosilicate, soda lime, or any other glass suitable for binding the polymer. Silicone polymers may include polydimethylsiloxane or any other silicone polymer suitable for binding the mixed-surface polymer. Metals may include gold, silver, platinum, or any other metal suitable for binding the mixed-surface polymer. Plastics may include a poly(methyl methacrylate), a poly(styrene), cyclic olefin copolymer, or any other plastic suitable for binding the mixed-surface polymer. Magnetic substrates may include any magnetic material suitable for binding the mixed-surface polymer, including, magnetic beads. The electrospun fiber may be any electrospun fiber suitable for binding the mixed-surface polymer, including those described in International Application Ser. No. PCT/US2015/026201 to Sullenger et al., published as WO2015/161094, which is incorporated by reference herein in its entirety. Those skilled in the art will appreciate that there may be many ways to immobilize the mixed-surface polymer to the substrate depending on the choice of substrate. Scavenging apparatuses may include those described in International Application Ser. No. PCT/US2016/060652 to Sullenger et al., published as WO2017079638, which is incorporated by reference herein in its entirety.

### Microfibers

In embodiments, the substrate is a microfiber. The microfiber may comprise a high-aspect-ratio polymeric core, the polymeric core comprising a blend of a first core polymer and a second core polymer, and the mixed-surface PAMAM polymer immobilized on the surface of the polymeric core. The microfibers are capable of sequestering or clearing certain solutes or particles from solution or components from multicomponent fluids. Examples of solutes and particles that can be sequestered or cleared include, but are not limited to, nucleic acids, proteins, polysaccharide, glycosaminoglycan, supramolecular assemblies, e.g., biomolecular complexes, microparticles, or exosomes. For example pathogen associated molecular patterns (PAMPs) and damage-associated molecular patterns (DAMPs) are biomolecules that may result in inflammation which can be cleared using the methods and compositions provided herein. As a result, these mixed-surface PAMAM microfibers may be used as filters or in filtration apparatuses. Moreover, these microfibers may be useful for preparing solute-cleared and/or particulate-cleared fluids and solutions that, in turn, may be useful for the treatment of certain diseases or conditions or for the prevention of certain diseases or conditions.

In embodiments, the polymeric core has a cross-sectional diameter between about 1.0 micron and about 1.0 millimeter. In particular embodiments, the polymeric cores have a cross-sectional diameter between about 2.0 microns to about 10.0 microns. The polymeric core may be electrically neutral. The polymeric core may comprise a blend of a first core polymer and a second core polymer, where the first core polymer and the second core polymer are different polymers. The first core polymer and/or the second core polymer may be a homopolymer or a copolymer. In some embodiments, the first core polymer is a copolymer and the second core polymer is a homopolymer. Copolymers may be an alternating copolymer, a block copolymer, or a random copolymer comprising a residue of a first monomer and a residue of a second monomer. In some embodiments, the first core polymer, the second core polymer, or both the first core polymer and the second core polymer have polar and/or hydrophilic residues. In some embodiments, the first core polymer, the second core polymer, or both the first core polymer and the second core polymer have apolar and/or hydrophobic residues. In certain embodiments where a core polymer is a copolymer, the first core polymer, the second core polymer, or both the first core polymer and the second core polymer have both (i) polar and/or hydrophilic residues and (ii) apolar and/or hydrophobic residues. In certain embodiments where a core polymer is a copolymer, the mixed surface polymers may be preferentially immobilized on the residue of the first monomer or the second monomer. In some embodiments, a residue of the first core polymer and a residue of the second core polymer are the same residue. In some embodiments, a residue of the first core polymer and a residue of the second core polymer are different residues.

In some embodiments, polar and/or hydrophilic monomers may be any polar and/or hydrophilic monomers capable of being polymerized. In particular embodiments, polar and/or hydrophilic monomers may be any of the following monomers: acrylic acid, acrylate, acrylamide, and maleic anhydride, allylamine, ethyleneimine, 2-ethyl-2-oxazoline, 2-methyl-2-oxazoline, vinyl alcohol, vinylpyrrolidone, ethylene glycol, propylene glycol, ethylene oxide, methacrylate, methacrylic acid, N-acryloylmorpholine, beta-carboxyethyl acrylate, diethylene glycol diacrylate, diethylene glycol dimethacrylate, ethylene Glycol Dimethacrylate, hydroxypolyethoxy (10) allyl ether, (HEMA 10) poly ethoxy (10) ethyl methacrylate, or sulfone.

In some embodiments, apolar and/or hydrophobic monomers may be any apolar and/or hydrophobic monomers capable of being polymerized. In particular embodiments, apolar and/or hydrophobic monomers may be any of the following monomers: styrene, stearyl acrylate, N-(n-Octadecyl) acrylamide, t-amyl methacrylate, butyl methacrylate, benzyl acrylate, decyl methacrylate, decyl acrylate, cyclohexyl acrylate, cyclohexyl methacrylate, bis(2-methacryloxyethyl)-N,N′-1,9-nonylene biscarbamate, 2,2-bis(4-methacryloxyphenyl) propane, or propylene.

A polymer blend may have any weight ratio of the first core polymer to the second core polymer that allows for the formation of a high-aspect-ratio microfiber. In certain embodiments, the weight ratio of the first core polymer to the second core polymer is about 5:1 to about 1:5. In particular embodiments, the weight ratio of the first core polymer to the second core polymer is about 4:1 to about 1:4, about 3:1 to about 1:3, about 2:1 to about 1:2, or about 1.5:1 to about 1:1.5.

The first core polymer and second core polymer may each be characterized by a weight-average molecular weight (Mw) or any other suitable weight, and the ratio of the Mw of the first core polymer to the Mw of the second core polymer may be any suitable ratio that allows for the formation of a high-aspect ratio microfiber. In certain embodiments, the ratio is about 3:1 to about 1:3. In particular embodiments, the ratio is about 2:1 to about 1:2 or about 1.5:1 to about 1:1.5. The microfiber may comprise a poly(styrene-alt-maleic anhydride) (PSMA) core polymer. The microfiber may comprise a polystyrene core polymer. The microfiber may comprise a first core polymer of PSMA and a second core polymer of polystyrene. Microfibers may include those described in International Application Ser. No. PCT/US2017/068262 to Lee et al., published as WO2018119422.

### Filter

Provided herein filters comprising the scavenger apparatus having a microfiber described herein, wherein the microfiber is formed into a porous mesh. The filter may be configured to bind or sequester a nucleic acid, a protein, a polysaccharide, a glycosaminoglycan, a biomolecular complex, an exosome, or a microparticle from a solution or a sample. In particular embodiments, the mesh is ordered, i.e., has a regular pattern. Ordered meshes may be prepared in a number of different ways, including by methods known in the 3-D printing arts and electrospinning. In other embodiment, the mesh is disordered, i.e., is amorphous in form. Filters may include those described in International Application Ser. No. PCT/US2017/068262 to Lee et al., published as WO2018119422.

### Extracorporeal Filtration Apparatus

Another aspect of the invention is extracorporeal filtration apparatuses. The extracorporeal filtration apparatuses allow for the establishment of an extracorporeal circuit for continuously removing a bodily fluid from a subject, sequestering some or all of a particular solute or particle from a volume of the bodily fluid to clear the solute or particle from the fluid, and to return the solute- or particle-cleared fluid to subject. The extracorporeal filtration apparatus may be useful for treating a subject suffering from a disease or a condition, for the prevention of the onset of a disease or a condition or for the prevention of graft injury, graft dysfunction, transplantation-associated inflammation and thrombosis and/or graft rejection. Extracorporeal filtration apparatuses include a first end of a line configured to interface with a subject configured for the removal of a bodily fluid, a second end of a line configured to interface with the subject configured for the return of a fluid to the subject, a pump positioned along the line between the first end and the second end configured for the extracorporeal circulation of the bodily fluid from the first end to the second and, and a filter positioned along the line between the first end and the second end configured to bind or sequester a solute present in the bodily fluid removed from the subject. The extracorporeal filtration apparatuses may comprise a first end of a blood line configured to interface with a first blood vessel of the subject for the removal of blood from the subject; a second end of the blood line configured to interface with a second blood vessel of the subject for the return of blood to the subject; a blood pump positioned along the blood line between the first end and the second end configured for the extracorporeal circulation of blood from the first end to the second end; and any of the filters described above positioned along the blood line between the first end and the second end configured. The extracorporeal filtration apparatus may further comprise a substitution solution pump for the introduction of a substitution solution to the line; an anticoagulant pump for the introduction of an anticoagulant to the line; a filtration pump for the removal of a filtrate from the line; a pressure monitor; a gas monitor; or any combination thereof.

Extracorporeal membrane oxygenation (ECMO) and hemofilters are often used in critically ill patients who have cardiac and pulmonary dysfunctions and who have a high risk of acute lung and kidney injuries. In a dog model, early treatments with continuous veno-venous hemofiltration (CVVH) significantly decreased mortality and acute lung injury in animals with severe drowning accident. The CVVH reduced the levels of circulating proinflammatory cytokines and oxidative stress in these animals. Moreover, blood purification by hemofilters is broadly used to remove circulating pathologic mediators from the patients with critical illness, such as severe sepsis and acute respiratory distress syndrome. Depending on surface modifications, hemofilters can remove specific molecules from patient's blood. Cytokine-absorbing hemofilters decreased circulating pro-inflammatory cytokines, heart rate, blood lactate level, intra-abdominal pressure and mortality rate in patients with severe acute pancreatitis. Fibers coated with endotoxin-binding polymyxin B or opsonin have been shown to remove endotoxin and pathogens from patient's blood and ameliorated sepsis and acute respiratory distress syndrome. However, no such hemofilters have been developed to remove DAMPs from the blood of patients with sterile inflammatory and thrombotic complications. Extracorporeal membrane oxygenation (ECMO) may be used for controlling coagulation, as described in International Application Ser. No. PCT/US2020/063389 to Sullenger et al., published as WO2021113696.

Mixed-surface PAMAM polymer-immobilized microfiber meshes may be used during ECMO, CVVH and continuous renal replacement therapy (CRRT) in intensive care units. Removing pro-inflammatory and pro-coagulative mediators from circulation is an unmet need in the treatment of critically ill patients. Polymer-immobilized microfiber meshes can be developed as safe and effective anti-inflammatory and anti-thrombotic therapeutics for the treatment of patients with traumatic injuries and the enhancement of grafts of organs derived from patients with traumatic injuries. DAMPs are potential biomarkers and attractive therapeutic targets for critically ill patients. Cellular disruption by trauma releases DAMPs that are one of the key linkers between tissue damage, inflammation and systemic inflammatory response syndrome (SIRS). DAMPs influence not only disease progress in primary injured sites but also facilitate dysfunction of other organs and systemic complications. Furthermore, negatively charged DAMPs, including hyaluronic acid, cell-free nucleic acids and heparan sulfate, released after allograft reperfusion induced inflammation and thrombosis, which have a negative impact on transplant outcomes and facilitate pulmonary dysfunction and graft-versus-host disease after allogeneic transplantation. Moreover, elevated circulating DAMPs were shown to correlate with the onset of septic shock and organ failure in patients with sepsis.

Certain types of DAMPs, including exDNAs and DNA-binding proteins (e.g., histone and HMGB1), are known to be potent pro-coagulants. Moreover, DAMP-stimulated TLRs on platelets and polymorphonuclear cells indirectly promote thrombosis.

Activation of TLR4 on platelets enhanced microvascular thrombosis in endotoxemia and trauma animal models. Patients with severe traumatic brain injury (TBI) have increased risk of disseminated intravascular coagulation. It has been demonstrated that TBI elevated the levels of circulating HMGB1 that caused systemic hypoxia, acute lung injury and pulmonary neutrophilia. Lung transplantation from donor mouse with TBI induced acute pulmonary dysfunction, and treatments with HMGB1 neutralizing antibody ameliorated this pulmonary dysfunction. Interestingly, in this study, a normal donor heart perfused with DAMPs developed acute thrombotic complications after transplantation, and treatment with NABP-immobilized microfiber meshes captured and removed DAMPs and prevented the development of thrombotic complications after transplantation. Therefore, treatment with NABP-immobilized microfiber meshes can reduce systemic DAMP levels and ameliorate inflammatory and thrombotic complications in trauma and transplantation. Extracorporeal filtration apparatuses may include those described in International Application Ser. No. PCT/US2017/068262 to Lee et al., published as WO2018119422.

### Ex Vivo Perfusion or Filtration Machine

Another aspect of the invention is an ex vivo perfusion and/or filtration machine. The machine may be used to reduce or prevent injury to a tissue, graft, or organ, avoiding dysfunction. This allows the tissue, graft, or organ to be available for transplantation into a subject. The machine may also be able to reduce inflammation, thrombosis, or rejection of a tissue, graft, or organ resulting from transplantation of the tissue, graft, or organ into a subject. In some embodiments, the machine is a hypothermic machine, a normothermic machine, or a subnormothermic machine.

An ex vivo perfusion or filtration machine comprises a housing configured to host the tissue, the graft, or the organ. The tissue, graft, or organ may be any tissue, graft, or organ suitable for perfusion. In some embodiments, the organ hosted by the machine is a liver, kidney, heart, lung, or any other organ suitable for perfusion. In some embodiments, the graft is a vascular composite allograft, including, but not limited to skin, muscle, bone, face, hand, leg, or any other vascular composite allograft suitable for perfusion. The machine further comprises a first end of a line configured to interface with a tissue, a graft, or a organ and allow for the introduction of a perfusate to the tissue, the graft, or the organ and a second end of the line configured to interface with the tissue, the graft, or the organ and allow for the removal of the perfusate from the tissue, the graft, or the organ. The first end may comprise a first cannula configured to introduce the perfusate to arterial vasculature. The second end may comprise a second cannula configured to remove the perfusate from venous vasculature. The machine further comprises a pump positioned along the line between the first end and the second end configured for the circulation of the perfusate from the second end to the first end. The machine further comprises a filter comprising a microfiber formed into a porous mesh positioned along the line between the first end and the second end, wherein the microfiber is configured to bind or sequester a nucleic acid, a protein, a polysaccharide, a glycosaminoglycan, a biomolecular complex, an exosome, or a microparticle in the perfusate. The microfiber may be any of the microfibers disclosed herein. The filter may be any of the filters described herein.

The perfusate may be a bodily fluid, preservation solution, or any other fluid suitable for perfusion of tissue, grafts, or organs. The bodily fluid may be blood, lymph, plasma, serum, cerebral spinal fluid, urine or any other bodily fluid. The preservation solution may be any organ preservation solution, including, but not limited to, UW solution, saline, or machine perfusion solution.

The machine may further comprise one or more components. For example, the machine may further comprise a perfusate pump for the introduction of perfusate to the line, an oxygenator to elevate the amount of dissolved oxygen in the perfusate, a filtration pump for the removal of a filtrate from the line, a pressure monitor, a gas monitor, a perfusate reservoir, or any combination of the components. Filtration machines may include those described in International Application Ser. No. PCT/US2017/068262 to Lee et al., published as WO2018119422.

Methods of Sequestering Molecules, Complexes, and/or Supramolecular Assemblies with Mixed-Surface PAMAM Polymer Microfibers

Another aspect of the invention is methods for scavenging solutes from a solution, a biological sample, a preservation solution, or a bodily fluid. The fluids following the sequestration of the solute may be referred to as a solute-cleared fluid. The solute may be any solute of interest capable of being sequestered by a mixed-surface polymer microfiber. In some embodiments, the solute is a nucleic acid, a protein, a polysaccharide, a glycosaminoglycan, a biomolecular complex, an exosome, or a microparticle.

The method comprises contacting the solution comprising a solute capable of being bound or sequestered by any of the scavenging apparatuses, microfibers, or filters described above. The solution may be artificially created by human intervention or a biological sample obtained from a subject or a patient. When the solution is a biological sample obtained from a subject or a patient, the solution may be blood, lymph, plasma, serum, cerebral spinal fluid, urine or any other bodily fluid. In certain embodiments, the solution may be organ preservation solution (e.g., UW solution, saline or machine perfusion solution). In certain embodiments, the solution or biological sample comprises cell-free nucleic acids, DAMPs, PAMPs, biomolecular complexes, exosomes, or microparticles.

In an alternative embodiment, the polymer is deposited into the solution or biological sample and the solution or sample comprising the polymer is contacted with the polymeric core. By depositing the polymer into the solution or biological sample, you allow for the formation of adjuncts between the polymer and solutes present. When the adjuncts are later contacted with the polymeric core, the adjuncts may bind to the polymeric core through either chemisorption or physiosorption. This, in turn, sequesters the solutes.

The solute-cleared fluid may have any amount of the solute of interest cleared from the fluid depending on the method for removing the solute and/or the intended use of the solute-cleared fluid. In some embodiments, more than 1% of the solute of interest is removed from the fluid. In certain embodiments, more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%), more than 85%, more than 90%, or more than 95% of the solute of interest is removed from the fluid.

### Methods of Using the Mixed-Surface Polymers for Therapeutic Applications

Another aspect of the invention includes methods of treating a subject suffering from a disease or a condition. The methods comprise administering a therapeutically effective amount of a solute-cleared fluid to the subject, wherein the solute-cleared fluid is prepared from a bodily fluid. In some embodiments, the solute-cleared fluid is administered continuously. This may be accomplished through the use of an extracorporeal filtration apparatus that removes a small portion of the subject's blood, clears some portion of a solute of interest, and then returns the solute-cleared fluid to the subject. Because the solute-cleared fluid is administered continuously, the amount of solute cleared from the bodily fluid may be small. In other embodiments, the solute-cleared fluid is administered by transfusion. Because transfusion delivers a quantum of solute-cleared fluid to the subject, it may be preferable for the amount of solute cleared from the fluid to be high.

The bodily fluid may be any bodily fluid comprising a solute capable of being sequestered by a microfiber. In some embodiments, the bodily fluid is blood, lymph, plasma, serum, cerebral spinal fluid, urine or any other bodily fluid. The bodily fluid may be removed from the subject. In other embodiments, the bodily fluid may originate from a donor.

The disease or condition may be any disease or condition that may be effectively treated by the administration of a solute-cleared fluid. In some embodiments, the disease or the condition is selected from the group consisting of an organ transplant donor, an organ transplant recipient, thrombosis, sepsis, inflammatory disease, autoimmune disease, cardiovascular disease, cancer, and patients with infection or trauma.

The solute-cleared fluid may be prepared by any suitable method. In some embodiments, the solute-cleared fluid is prepared by contacting the bodily fluid with a microfiber as described above. In some embodiments, the solute-cleared fluid is prepared by contacting the bodily fluid with a filter as described above. In some embodiments, the solute-cleared fluid is prepared by contacting the bodily fluid with an extracorporeal filtration apparatus as described above.

Methods of treating cancer in a subject by administering a therapeutically effective amount of a polymer to the subject are provided herein. The polymer may be administered by any means known to those of skill in the art and may be administered in combination with other cancer therapeutic agents or in conjunction with or after surgical resection of the cancer. The polymer and cancer therapeutic agents may be combined in a single dosage form or may be administered in any order or concurrently via separate means. The polymer may be administered intraperitoneally, intratumorally or intravenously.

Methods of treating cancer in a subject using the mixed-surface PAMAM polymers and scavenging apparatuses described herein to remove invasion-promoting or metastatic molecules or particles from a bodily fluid of the subject are also provided. The invasion-promoting or metastatic molecules or particles include cell free DNA (cfDNA), cfRNA, microRNA, exosomes, microparticles or other particles such as nucleosomes or mitochondria, that may be inflammatory, may aid spread of the cancer cells to other locations or organs within the body or may be immunomodulatory to allow cancer cells to evade the immune response. The polymer may be immobilized on a substrate to allow for binding and removal of the invasion-promoting or metastatic molecules or particles from the body of the subject. Alternatively, the polymer may be made into electrospun nanofibers as described in International Publication Number WO/2015/161094. In one embodiment these molecules or particles may be found in a bodily fluid of the subject such as the blood or circulation of the subject and the polymer may inhibit the function of these or may bind to and allow removal of these molecules or particles from the bodily fluid. The bodily fluid may be selected from the blood, plasma, serum, cerebral spinal fluid, urine, saliva and lymph. The bodily fluid may be contacted with the polymer in vivo, such as on a surgical or other mesh or by soluble administration of the polymer to the subject. Alternatively, the bodily fluid may contact the polymer ex vivo, such as via a dialysis style procedure in which the subject's blood or sera from the patient is brought into contact with the polymer and then returned to the subject.

The polymer may be immobilized on a substrate. The bodily fluid may be brought into contact with the substrate. This contact may occur in vitro, in vivo or ex vivo. For in vivo contact the substrate may be placed near the tumor site or in a position within the body of the subject where the substrate and polymer are allowed to come into contact with bodily fluids. Alternatively, the bodily fluid may be carried from the body through an apparatus comprising the substrate to allow contact between the bodily fluid and the substrate similar to a dialysis machine. Finally the bodily fluid may be removed from the body, incubated for a period of time with the substrate and then returned to the subject. Those of skill in the art may envision other methods for contacting the bodily fluid with the substrate. The substrate may include a glass, silicon, a silicon polymer, a metal, a plastic, magnetic, or an electrospun fiber. Glasses may include silica, a borosilicate, or soda lime. Silicone polymers may include polydimethylsiloxane. Metals may include gold, silver, or platinum. Plastics may include a poly(methyl methacrylate), a poly(styrene), or cyclic olefin copolymer.

Contacting encompasses administration to a cell, tissue, mammal, patient, or human. Further, contacting a cell includes adding the polymer or substrate to a cell culture. Other suitable methods may include introducing or administering the polymer or substrate to a cell, tissue, mammal, or patient using appropriate procedures and routes of administration.

Treating cancer includes, but is not limited to, reducing the number of cancer cells or the size of a tumor in the subject, reducing progression of a cancer thereby making it a less aggressive form, reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc.

The cancer being treated is suitably a cancer prone to metastasis. The cancer may be selected from the group consisting of pancreatic, ovarian, colon, liver, brain, breast, prostate, bladder, melanoma, head, neck and lung cancers. Administration of PAMAM was able to reduce the mortality of pancreatic cancer in mice in the Examples. In particular this reduction in mortality was associated with a decrease in liver invasion of the tumor cells. In one embodiment the metastatic growth of the cancer is inhibited by administration of a polymer. Without being limited by theory the polymer may be blocking invasiveness of the cancer cells by binding cfDNA, binding microparticles or binding exosomes and blocking the activity of these particles or molecules in promoting invasiveness of the cancer cells.

Another aspect of the invention provides methods for the reduction of cell-free nucleic acid or other inflammatory mediators in a bodily fluid of a subject or patient having an abnormally high concentration of the cell-free nucleic acid or other mediator of inflammation in the bodily fluid. The method comprises contacting the bodily fluid with the mixed-surface PAMAM polymer or scavenging apparatus, wherein the contacting step reduces the concentration of the cell-free nucleic acid, DAMPS, PAMPS or other inflammatory mediators in the bodily fluid. The contacting step may be performed within the subject or the patient. The contacting step may be performed outside the subject or the patient, and the method may further comprise obtaining the bodily fluid from the patient and/or returning the bodily fluid to the subject or the patient. The methods may be performed using a procedure or machine such as hemodialysis or extracorporeal membrane oxygenation (ECMO). The bodily fluid may be blood, plasma, serum, cerebral spinal fluid, lymph, or any other bodily fluid having cell-free nucleic acids or other inflammatory mediators.

The subject or patient suffers from a condition associated with the abnormally high concentration of the cell-free nucleic acid or other inflammatory mediators. The condition may be a cancer, an effect associated with radiation therapy, an autoimmune disease, an infectious disease, or any other condition associated with abnormally high concentrations of cell-free nucleic acid or other inflammatory mediators in a bodily fluid. Practicing the methods described herein may provide therapeutic benefit for the subject or patient suffering from the condition.

Pharmaceutical compositions comprising the polymers described above and a pharmaceutically acceptable carrier are provided. A pharmaceutically acceptable carrier is any carrier suitable for in vivo administration. Examples of pharmaceutically acceptable carriers suitable for use in the composition include, but are not limited to, water, buffered solutions, glucose solutions, or oil-based carriers. Additional components of the compositions may suitably include, for example, excipients such as stabilizers, preservatives, diluents, emulsifiers and lubricants. Examples of pharmaceutically acceptable carriers or diluents include stabilizers such as carbohydrates (e.g., sorbitol, mannitol, starch, sucrose, glucose, dextran), proteins such as albumin or casein, protein-containing agents such as bovine serum or skimmed milk and buffers (e.g., phosphate buffer). Especially when such stabilizers are added to the compositions, the composition is suitable for freeze-drying or spray-drying. The composition may also be emulsified.

The polymer may be administered with a cancer therapeutic or radiation. The polymer and cancer therapeutics may be administered in any order, at the same time or as part of a unitary composition. The two may be administered such that one is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more. The polymer may be administered or used to contact a bodily fluid of the subject in conjunction with or after surgery to remove at least a portion of the cancer, such as tumor resection or tumor biopsy. Any cancer therapeutic known to those of skill in the art may be used in combination with the polymers. Suitable cancer therapeutics include, but are not limited to 5-fluorouracil, gemcitabine, cisplatin, capecitabine, paclitaxel, irinotecan, oxaliplatin, docetaxel, and erlotinib.

An effective amount or a therapeutically effective amount as used herein means the amount of the polymer that, when administered to a subject for treating cancer is sufficient to effect a treatment (as defined above). The therapeutically effective amount will vary depending on the compositions or formulations, the disease and its severity and the age, weight, physical condition and responsiveness of the subject to be treated.

The polymers, apparati, and compositions described herein may be administered by any means known to those skilled in the art, including, but not limited to, oral, topical, intranasal, intraperitoneal, parenteral, intravenous, intramuscular, subcutaneous, intrathecal, transcutaneous, nasopharyngeal, intratumoral, intrathecal, or transmucosal absorption. Thus the compounds may be formulated as an ingestible, injectable, topical or suppository formulation. The compositions may also be delivered within a liposomal or time-release vehicle. Administration to a subject in accordance with the invention appears to exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage administered in any given case will be adjusted in accordance with the compositions being administered, the disease to be treated or inhibited, the condition of the subject, and other relevant medical factors that may modify the activity of the compound or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compound of the invention and of a known agent such as tocopherol, such as by means of an appropriate conventional pharmacological or prophylactic protocol. The subject may be a human subject, a human suffering from cancer or a non-human animal subject. For example, the subject may be a domesticated animal such as a cow, pig, chicken, horse, goat, sheep, dog or cat.

The maximal dosage for a subject is the highest dosage that does not cause undesirable or intolerable side effects. The number of variables in regard to an individual prophylactic or treatment regimen is large, and a considerable range of doses is expected. The route of administration will also impact the dosage requirements. It is anticipated that dosages of the compositions will reduce symptoms of the condition at least 10%, 20%, 30%, 40%, 50%, 60%, 70%, 80%, 90% or 100% compared to pre-treatment symptoms or symptoms is left untreated. It is specifically contemplated that pharmaceutical preparations and compositions may palliate or alleviate symptoms of the disease without providing a cure, or, in some embodiments, may be used to cure the disease or disorder.

Suitable effective dosage amounts for administering the compositions may be determined by those of skill in the art, but typically range from about 1 microgram to about 500 milligrams per kilogram of body weight weekly, although they are typically about 50,000 micrograms or less per kilogram of body weight weekly. Large doses may be required for therapeutic effect and toxicity of the compositions is likely low. In some embodiments, the effective dosage amount ranges from about 10 to about 50,000 micrograms per kilogram of body weight weekly. In another embodiment, the effective dosage amount ranges from about 100 to about 25,000 micrograms per kilogram of body weight weekly. In another embodiment, the effective dosage amount ranges from about 1000 to about 20,000 micrograms per kilogram of body weight weekly. The effective dosage amounts described herein refer to total amounts administered, that is, if more than one compound is administered, the effective dosage amounts correspond to the total amount administered. The compositions can be administered as a single dose or as divided doses. For example, the composition may be administered two or more times separated by 4 hours, 6 hours, 8 hours, 12 hours, a day, two days, three days, four days, one week, two weeks, or by three or more weeks.

Methods of detecting cancer or detecting metastasis of cancer are also provided herein. The methods of detecting cancer include obtaining a sample from a subject and determining a level of circulating free DNA (cfDNA), cfRNA, inorganic polyphosphates, exosomes or nucleosomes in the sample. An increase in the level or number of any of these markers in the subject as compared to the level of the corresponding marker in a control subject without cancer is indicative of cancer in the subject. The control may be an individual without cancer or may be a sample obtained from the subject being tested at a point when they were cancer free. For example, a subject may routinely be screened for the level or number of these markers present in their serum each year at an annual physical. The subject's normal levels of these markers in the serum then can become their own control level and increased levels may be detected relative to the same subject. Alternatively, a standard level may be generated such that a subject is compared to a standard control level or number of the markers found in a healthy (non-cancer) set of individuals. When the level of cfDNA, cfRNA, inorganic polyphosphates, exosomes or nucleosomes is increased 20%, 25%, 30%, 35%, 40%, 45%, 50%, 55%, 60%, 70%, 75%, 80%, 90%, or more than 100% compared to the control, the subject may be diagnosed as having a high likelihood of having cancer and can then be subjected to further more invasive or more expensive testing to determine the type and location of the cancer. The number of nucleosomes may be indicative of cancer if the number increases 0.25 log, 0.5 log, 0.75 or 1 log as compared to the control.

Methods of detecting metastasis or recurrence of a cancer in a subject may include obtaining more than one sample from a subject diagnosed with cancer over a period of time. The period of time may extend from the point of diagnosis of the cancer throughout treatment and may extend beyond treatment of the cancer. For example, samples may be collected and analyzed prior to diagnosis of the cancer, at or shortly after diagnosis of the cancer, intermittently during treatment of the cancer, during or after surgery to remove the cancer, after treatment is completed and at intermittent points thereafter to monitor for recurrence. The level of circulating free DNA (cfDNA), cfRNA, inorganic polyphosphates, exosomes or number of nucleosomes in each of the samples is determined and compared over time in the samples. If the level of any of these markers in the samples increases over time, it is indicative of metastasis or advancing cancer. In each of these methods, if cancer is detected or metastasis is determined to be likely based on increased levels or numbers of any of the markers, then a polymer may be administered to the subject as described above. The samples for use in these methods of detecting cancer or metastasis may be any bodily fluid including but not limited to blood, serum, plasma, saliva, cerebral spinal fluid and urine samples. In one embodiment of these methods of detecting cancer or metastasis, the polymer is administered to the subject prior to obtaining the sample or is added to the sample when collected form the subject. By administering a polymer or contacting the sample with the polymer, we can protect cfDNA from degradation during isolation and characterization as well as expand the analysis to cfRNA and inorganic polyphosphates using this protection approach. This may offer more sensitive detection of these markers in a subject and be useful for increasing the sensitivity and specificity of such methods.

Methods of using the mixed-surface PAMAM polymers, fibers, apparati, and compositions disclosed herein for inhibiting formation of a biofilm or microbial growth are also provided. The polymers may bind to microbes and inhibit microorganism proliferation, wound infection, and/or biofilm formation. The polymers and compositions may be incorporated into a medical device, filter, bandage, dressing, graft, etc. Anticoagulants may be applied with the polymers.

### Methods for the Prevention of a Disease or a Condition

Another aspect of the invention is methods of prevention of a disease or a condition in a subject. The methods comprise administering a prophylactically effective amount of a solute-cleared fluid to the subject, wherein the solute-cleared fluid is prepared from a bodily fluid. In some embodiments, the solute-cleared fluid is administered continuously. This may be accomplished through the use of an extracorporeal filtration apparatus that removes a small portion of the subjects blood, clears some portion of a solute of interest, and then returns the solute-cleared fluid to the subject. Because the solute-cleared fluid is administered continuously, the amount of solute cleared from the bodily fluid may be small. In other embodiments, the solute-cleared fluid is administered by transfusion. Because transfusion delivers a quantum of solute-cleared fluid to the subject, it may be preferable for the amount of solute cleared fluid to be high. The bodily fluid may be any bodily fluid comprising a solute capable of being sequestered by a fiber. In some embodiments, the bodily fluid is blood, lymph, plasma, serum, cerebral spinal fluid, urine or any other bodily fluid. The bodily fluid may be removed from the subject. In other embodiments, the bodily fluid may originate from a donor.

The disease or condition may be any disease or condition that may be effectively treated by the administration of a solute-cleared fluid. In some embodiments, the disease or the condition is selected from the group consisting of an organ transplant donor, an organ transplant recipient, thrombosis, sepsis, inflammatory disease, autoimmune disease, cardiovascular disease, cancer and patients with infection or trauma. In a particular embodiment, the subject is an organ recipient and the disease or the condition is thrombosis. The solute-cleared fluid may be prepared by any suitable method. In some embodiments, the solute-cleared fluid is prepared by contacting the bodily fluid with a microfiber as described above. In some embodiments, the solute-cleared fluid is prepared by contacting the bodily fluid with a filter as described above. In some embodiments, the solute-cleared fluid is prepared by contacting the bodily fluid with an extracorporeal filtration apparatus as described above.

The microfibers described herein may be contacted with cells or tissues directly or indirectly in vivo, in vitro, or ex vivo. Contacting encompasses administration to a cell, tissue, mammal, patient, or human. Further, contacting includes adding the microfibers to a cell culture to a wound site or site of inflammation or to a solution. Other suitable methods may include introducing or administering the microfibers to a solution, cell, tissue, mammal, or patient using appropriate procedures and routes of administration as defined below. The microfibers may be administered to a subject.

Administration includes topical, subcutaneous, transcutaneous or any other means of bringing the microfibers in contact with the subject and the site of inflammation, infection or other site at which anionic compounds need to be adsorbed. The microfibers described herein may be administered in an amount and way such that the microfibers are in an effective amount to treat a condition, such as inflammation, infection or reversal of the effects of an anionic compound. An effective amount or a therapeutically effective amount as used herein means the amount of the nanofibers that, when administered to a subject for treating a state, disorder or condition is sufficient to effect a treatment. The therapeutically effective amount will vary depending on the compound, formulation or composition, the disease and its severity and the age, weight, physical condition and responsiveness of the subject to be treated. Treating a subject as used herein refers to any type of treatment that imparts a benefit to a subject afflicted with a disease or a condition or at risk of developing the disease or condition, including improvement in the condition of the subject (e.g., in one or more symptoms), delay in the progression of the disease or condition, delay the onset of symptoms or slow the progression of symptoms, etc.

### Miscellaneous

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

We hypothesized that we could retain the scavenging properties of PAMAM and mitigate its toxicity by diluting the density of the positive surface charge of cationic PAMAM with neutral moieties. To investigate this possibility, we designed PAMAM generation 3 and 4 variants with a 50:50 mix of cationic and neutral surface groups and examined their scavenging function as well as their cellular and in vivo toxicity. Understanding the contribution of size and charge density to the scavenging function and toxicity profile of PAMAM will enable rational design of biomaterials which retain anti-inflammatory properties but have less toxicity. The design of such agents will help realize the goal of safe and effective biomaterial-based therapy for lupus and other inflammatory diseases.

### Results:

### Effect of Polycationic Polymers on Activation of TLR Receptors

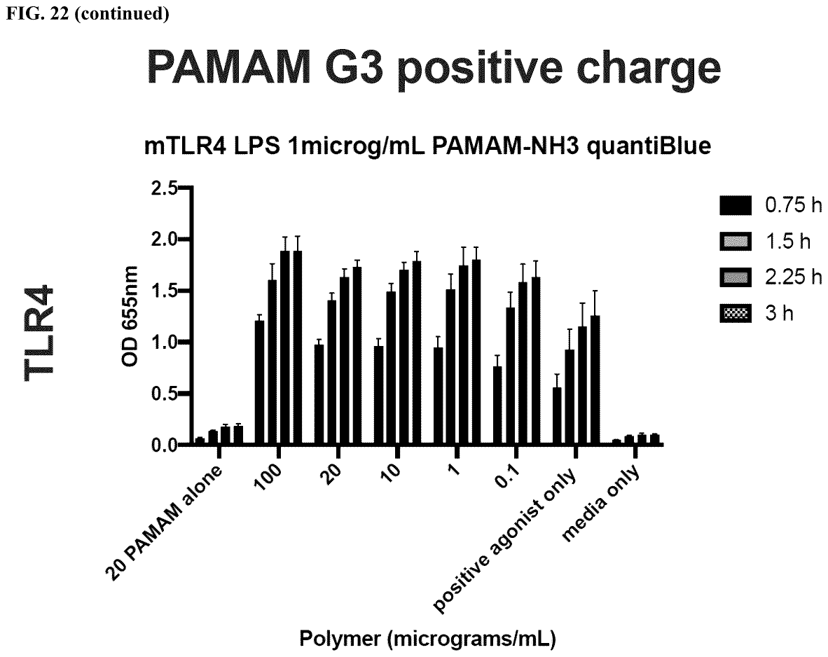

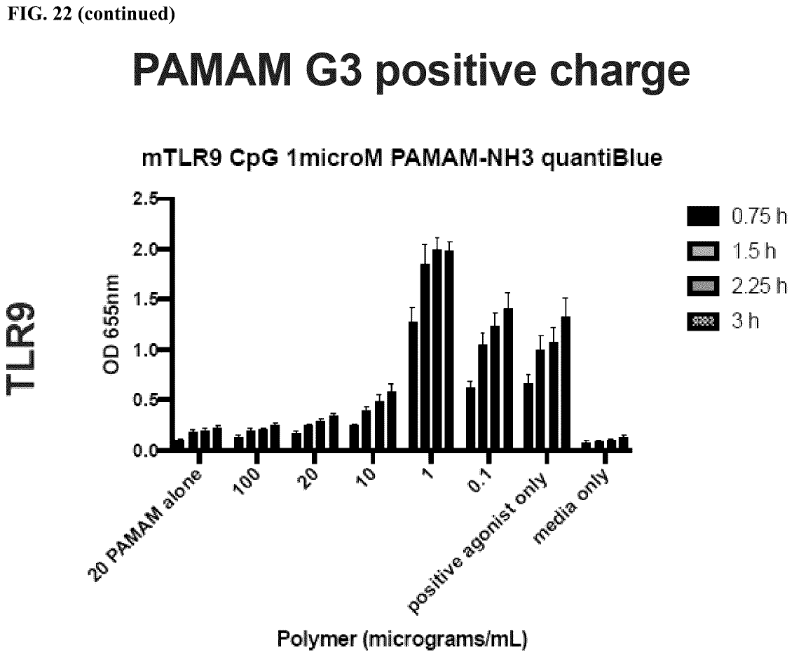

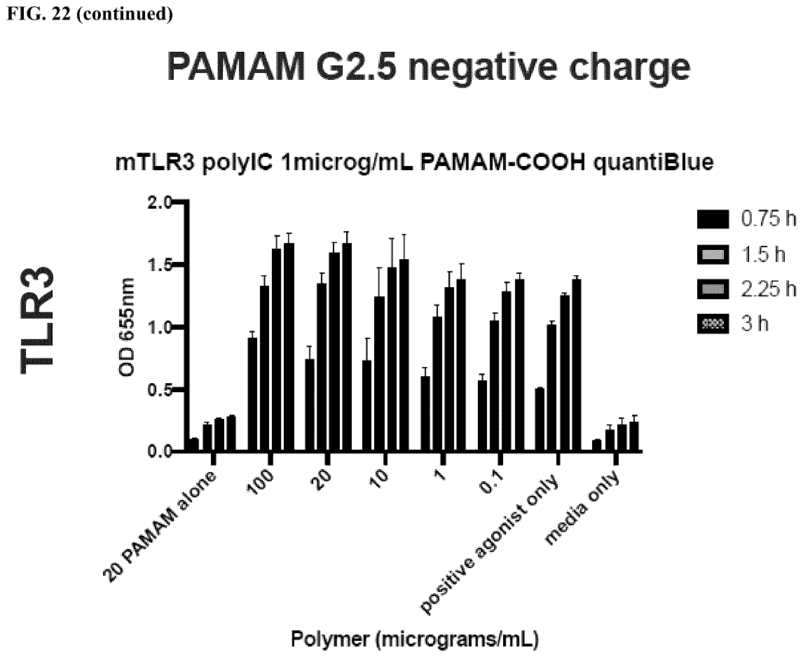

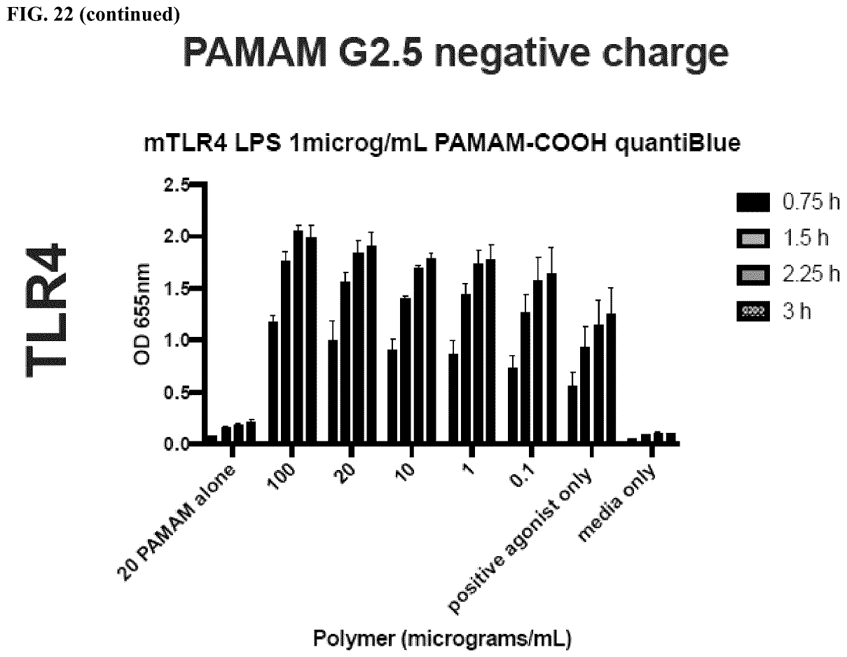

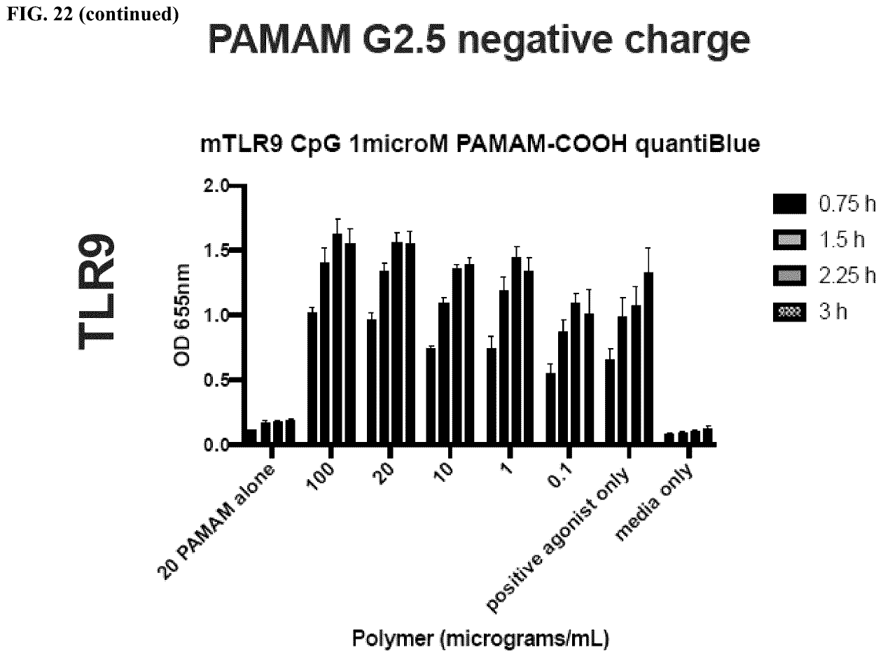

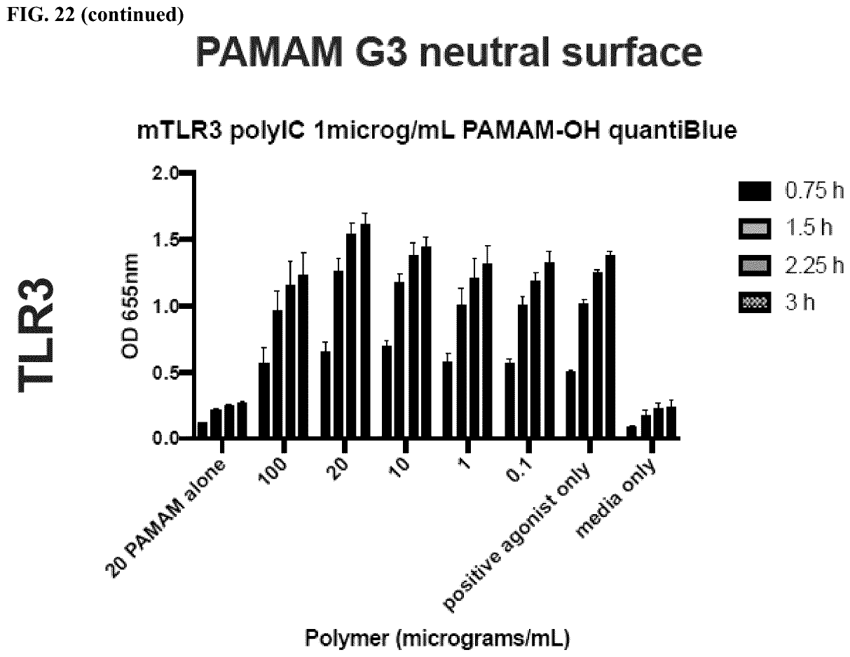

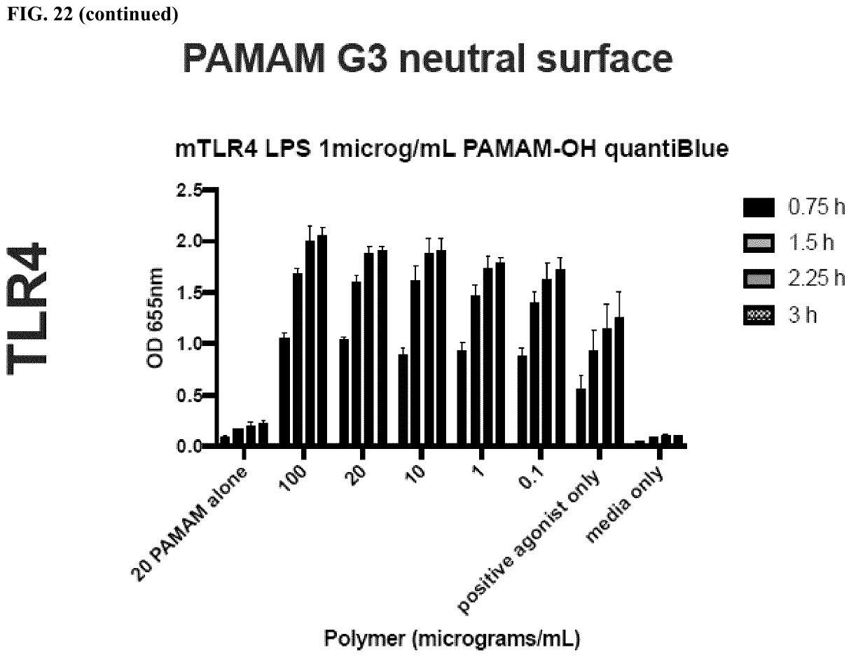

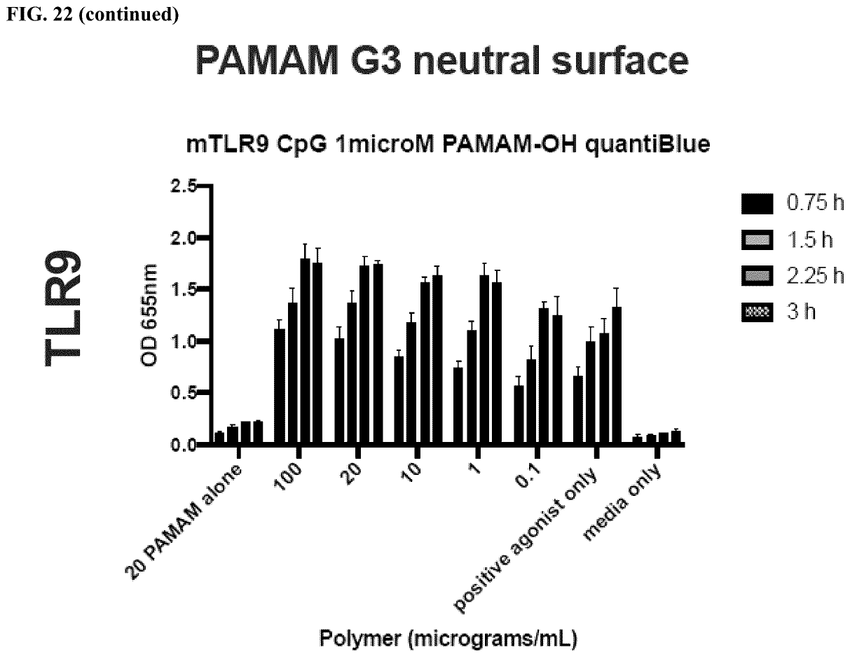

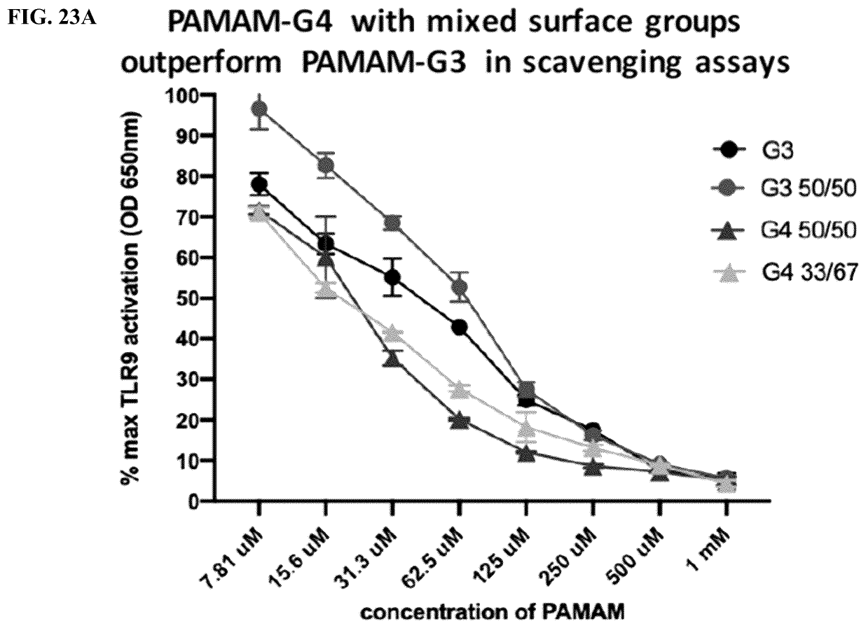

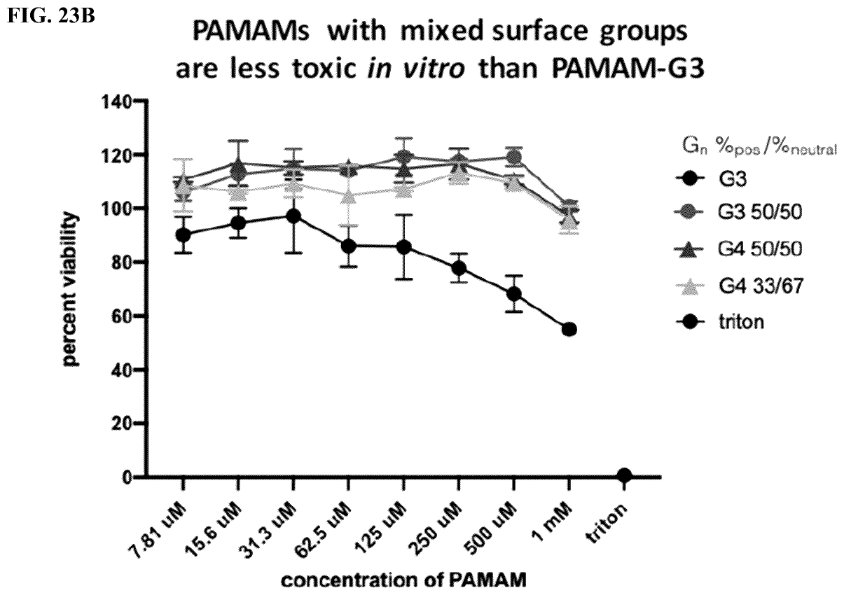

As shown in FIG. 22, PAMAM G3 positive charge is able to prevent activation of TLR3 and TLR9, but not TLR4 receptors, presumably by scavenging of negatively charged TLR3 and 9 agonists. (Left-most column of graphs, comparing polymer+agonist (columns 2-5/6) to positive agonist only columns (column 7)). In contrast, PAMAM G2.5 negative (middle column) and G3 neutral (Right column) do not limit activation of TLR receptors in the presence of canonic agonists.

Mixed Surface PAMAM Polymers Feature Neutral Hydroxyl Groups that Disperse the Cationic Moieties Arrayed on their Surface

The design of the mixed surface polymers, in comparison to PAMAM G3, is shown in FIG. 16. The mixed polymer PAMAM G3 50:50 features 16 amine groups and 16 hydroxyl groups compared to the 32 amine groups on PAMAM G3's surface. The higher generation mixed polymer PAMAM G4 50:50 features 32 surface amine groups, as PAMAM G3 does, but they are interspersed with neutral hydroxy groups over a larger surface area. Other fully cationic PAMAMs besides PAMAM G3 were included for comparison in certain studies; these compounds include the smaller PAMAM G2 with 16 amine groups and the larger PAMAM G4 with 64 amine groups.

### Mixed Surface PAMAM Variants Scavenge Nucleic Acid-Based TLR Agonists

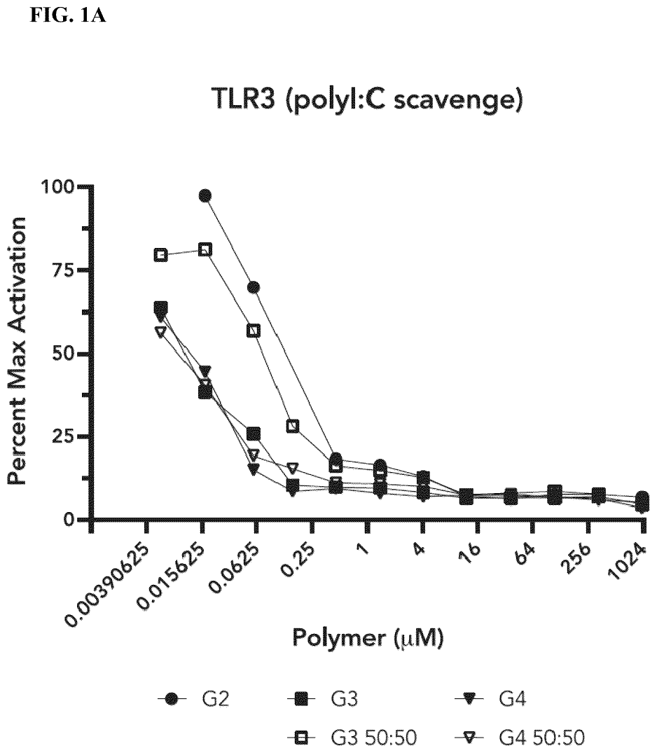

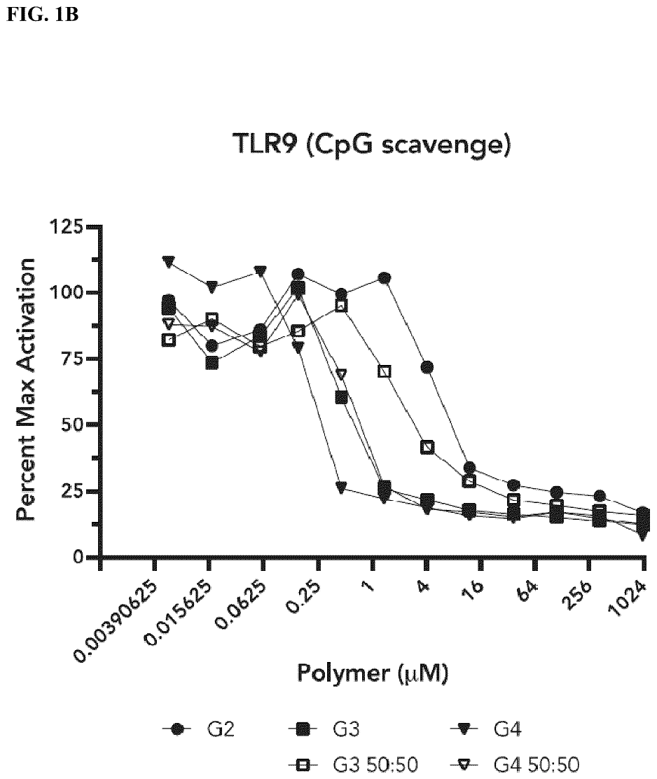

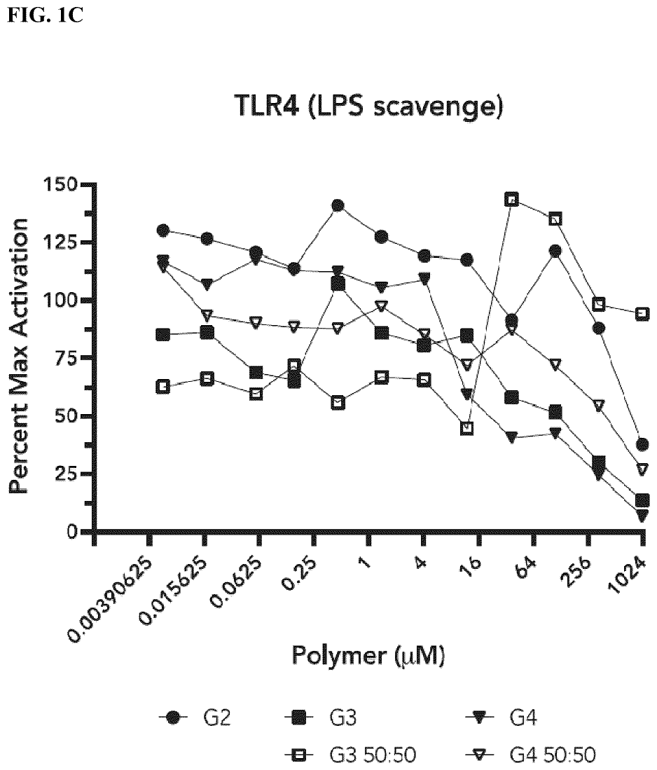

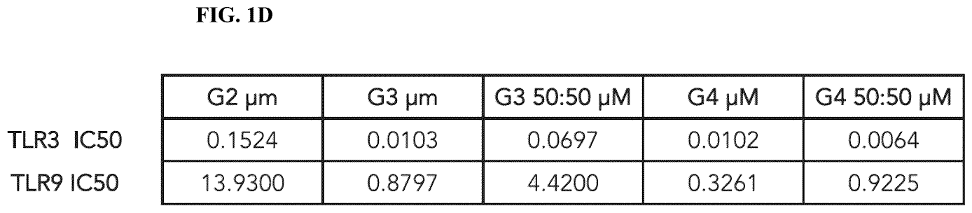

To justify further characterization, the mixed surface polymers should demonstrate a similar capacity for scavenging nucleic acid-based DAMPs/PAMPs as their fully cationic precursors. Therefore, the nucleic acid scavenging capacity of the mixed surface polymers was tested alongside the fully cationic PAMAMs. The prototypic DAMPs used were polyI: C, a synthetic analog of double-stranded RNA that mimics viral infection by stimulating TLR3, and CpG 1668, a modified DNA oligonucleotide containing an unmethylated CpG dinucleotide motif found enriched in bacterial genomes and mitochondrial DNA, that acts through TLR9. PAMAM G3 50:50 and G4 50:50 prevented TLR3 activation by polyI: C (FIG. 1A) and TLR9 activation by CpG in a dose responsive manner (FIG. 1B) as well as or better than the fully cationic PAMAM variant with the same number of cationic end groups (e.g. compare PAMAM G3 50:50 to PAMAM G2). The concentrations at which the respective polymers inhibited TLR3 and TLR9 activation by 50% (IC50 values) are shown in FIG. 1D. For TLR3 signaling inhibition, for instance, about 7 times more G3 50:50, 15 times more G2, and similar amounts of G4 and G4 50:50 were required compared to the benchmark polymer G3. As expected, none of the cationic or mixed-surface polymers was able to prevent TLR4 activation by the non-nucleic acid agonist, lipopolysaccharide LPS-B5 except at extremely high concentrations, demonstrating the nucleic acid-DAMP/PAMP selectivity of the inhibition (FIG. 1C).

PAMAM G3 and Mixed Surface PAMAM Variants Bind Nucleic Acids with Similar Thermodynamic Parameters

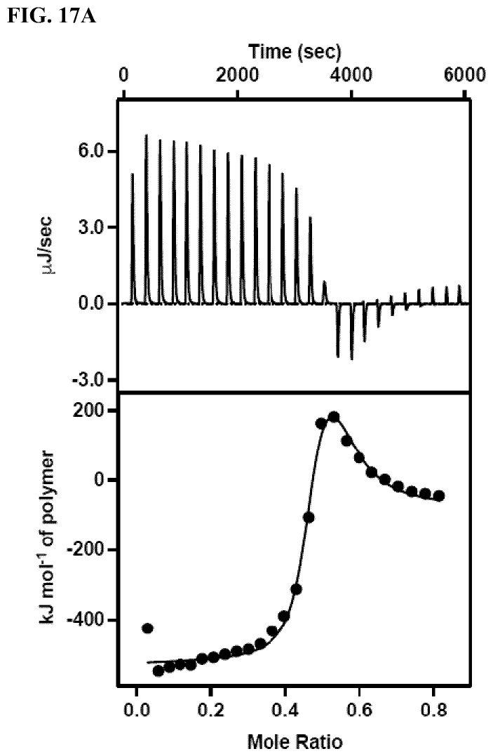

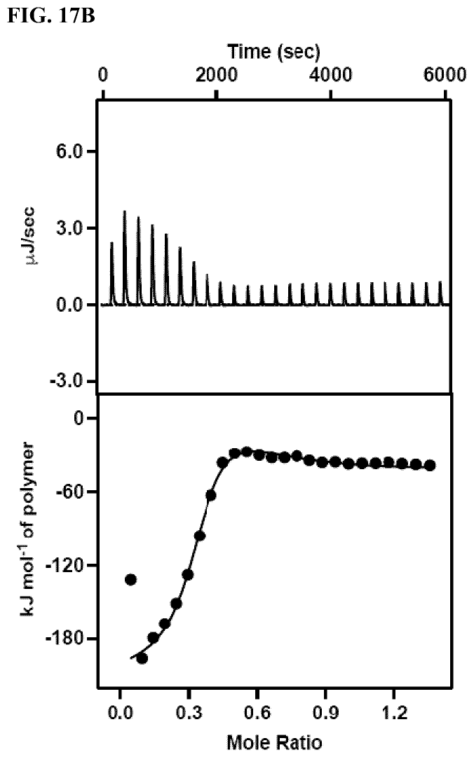

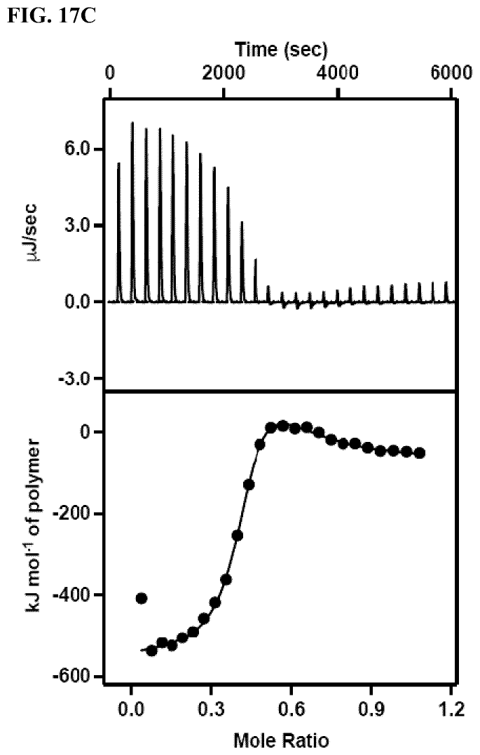

Isothermal Titration calorimetry (ITC) was employed to assess the nucleic acid binding thermodynamics and stoichiometry of the PAMAM polymers bearing different surface cation numbers and densities. Specifically, PAMAM G3, G3 50:50, and G4 50:50 dendrimers were titrated into a solution of CpG 1668 oligonucleotide in PBS, and a separate titration of each polymer to PBS was used to correct the dissolution heat. The raw heat data, corrected for dilution and integrated, yielded thermograms with a biphasic pattern suggestive of a two-site binding mechanism between PAMAM and CpG (FIG. 17). The thermograms were therefore fitted using an independent two-site binding model. The best-fit values for the associated thermodynamic parameters are shown in Table 1. The result showed that all polymers have similar binding stoichiometries and binding mechanisms; the first set of binding sites can bind 2-3 CpGs per polymer. This ratio is expected because the 16-32 amine groups on each polymer can simultaneously interact with negatively charged phosphate groups on different CpG molecules. The binding stoichiometry is not a whole number, possibly related to a non-specific binding mechanism wherein the capacity of each polymer molecule cannot be well-defined. As expected, this interaction between polymers and CpG is enthalpy favored (ΔH<0) due to the electrostatic interaction formed during binding. At the same time, entropy is unfavored due to the limitation of free movements of the molecules upon binding. Interestingly, despite different sizes, cation numbers, and cation densities, the three polymers did not significantly differ in CpG binding capacity. On the other hand, the dissociation constants varied over a forty-fold range. PAMAM G3, with a higher cation density, demonstrated the highest binding affinity for the DNA oligonucleotide (K_{D}=4.32 nM), while PAMAM G3 50:50 possessing both lower cation number and density showed the lowest binding affinity (K_{D}=168 nM). Nonetheless, all three polymers have nanomolar affinities to the CpG oligonucleotide which is consistent with their common ability to scavenge DNA and RNA (FIG. 1). The second set of binding sites for each polymer showed an unrealistically high binding capacity (≥10 CpG per polymer) and is unexpectedly entropy-driven. We surmise that this binding process represents the non-specific adsorption of CpG to impurities in the polymer preparation or the formation of higher order complexes. Elimination of such impurities or complex formation at high concentrations is difficult to accomplish due to the heterogeneous nature of polymer synthesis and spontaneous aggregation of the polymers in storage. We, therefore, omit further analysis of this binding site.

TABLE 1
Thermodynamic parameters of first site binding and second site binding between each
polymer and the CpG 1668 oligonucleotide. For each polymer, isothermal titration
calorimetry results were corrected for the heat of dilution and the integrated heat
data were fitted with a two-site binding model in the program Nano Analyze. The best
fit parameters resulting from the nonlinear regression fit of the data are shown.
(n: stoichiometric ratio of polymer to the CpG oligonucleotide, Kd (M): dissociation
constant, ΔH (kJ/mol): enthalpy change, ΔS (J/mol · K): entropy change)

| First Site: | n1 | Kd1 | ΔH1 (kJ/mol) | ΔS1 (J/mol · K) | CpG/Polymer |
|---|---|---|---|---|---|
| PAMAM G3 | 0.449 | 4.32E−9 | −428 | −1280 | 2.23 |
| PAMAM G3 50:50 | 0.340 | 1.68E−7 | −149 | −371 | 2.94 |
| PAMAM G4 50:50 | 0.411 | 4.602E−8 | −444 | −1350 | 2.43 |
| Second Site: | n2 | Kd2 | ΔH2 | ΔS2 | CpG/Polymer |
| PAMAM G3 | 0.100 | 3.39E−7 | 742 | 2610 | ≥10 |
| PAMAM G3 50:50 | 0.100 | 4.07E−6 | 436 | 1570 | ≥10 |
| PAMAM G4 50:50 | 0.100 | 1.66E−6 | 1070 | 3710 | ≥10 |

Mixed Surface PAMAM Variants Bind Native Double-Stranded DNA and Inhibit Recognition of that DNA by an Anti-DNA Antibody

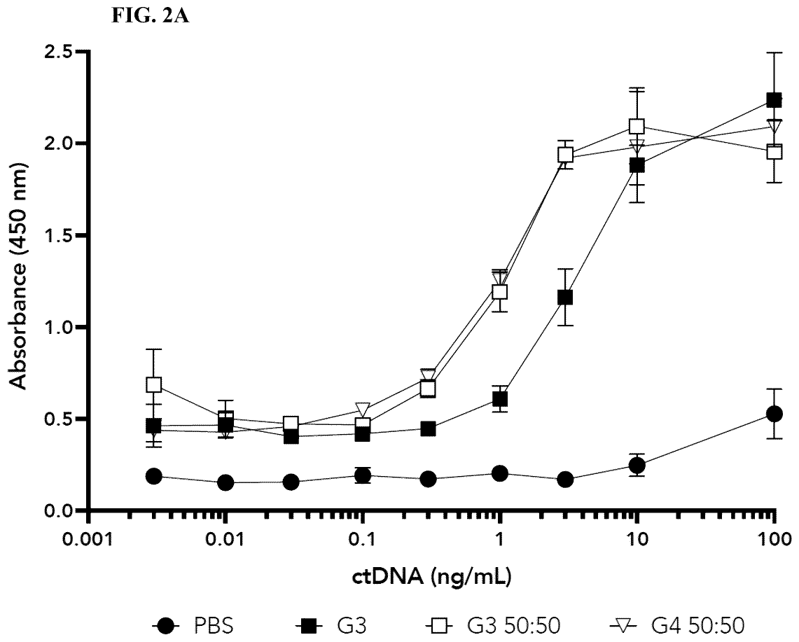

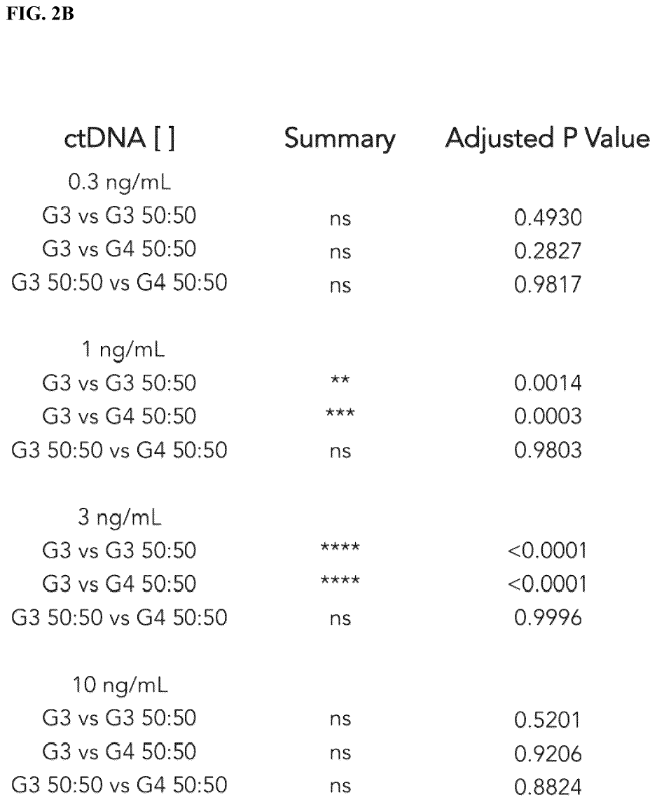

PAMAM G3 has been shown to bind DNA and inhibit the binding of anti-DNA antibodies, which is likely a factor in its effects in lupus-prone animals (Stearns, Lee et al. 2012, Holl, Shumansky et al. 2016). The mixed surface variants were compared to PAMAM G3 in enzyme-linked immunosorbent assays (ELISAs) that assessed the capture of double-stranded calf thymus DNA (ctDNA) and the inhibition of anti-DNA antibody binding to ctDNA. In the ELISA shown in FIG. 2, equal amounts by weight of each polymer (100 ng: 14.5 μmol G3, 14.4 μmol G3 50:50 and 7.0 μmol G4 50:50) were used to coat each well, a dilution series of ctDNA was applied, followed by washing and detection of captured ctDNA using the anti-DNA antibody Val-1205. The mixed surface polymers generated a dose-responsive signal with ctDNA concentrations at 0.3 ng/ml and greater while the fully cationic G3 required at least 1 ng/mL ctDNA for signal and was consistently less efficient in capturing DNA than the G3 50:50 variant. Selected results from a two-way ANOVA analysis shown in FIG. 2B reveal that between baseline signal and peak signal, the mixed polymers bind and capture DNA significantly better than parental PAMAM G3 in this ELISA format.

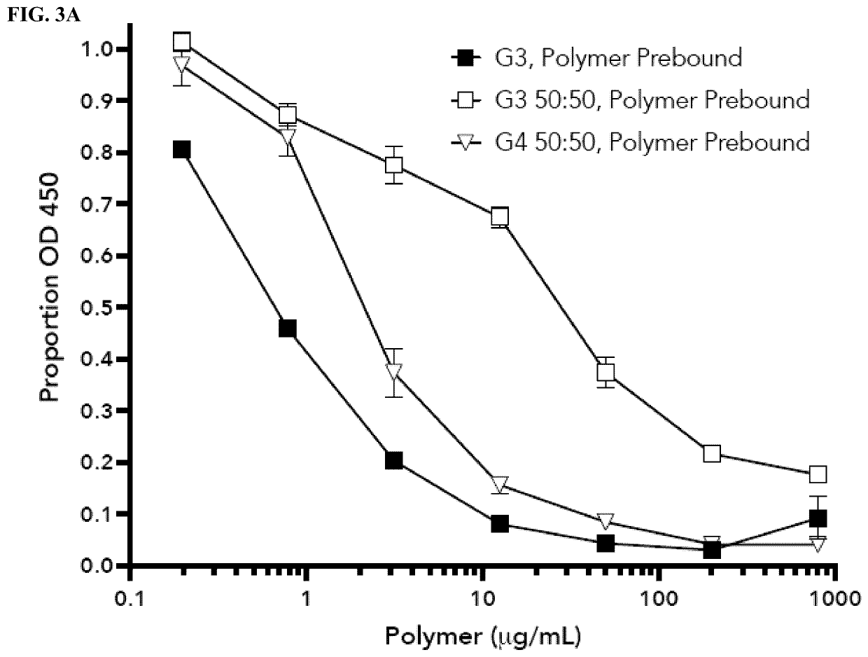

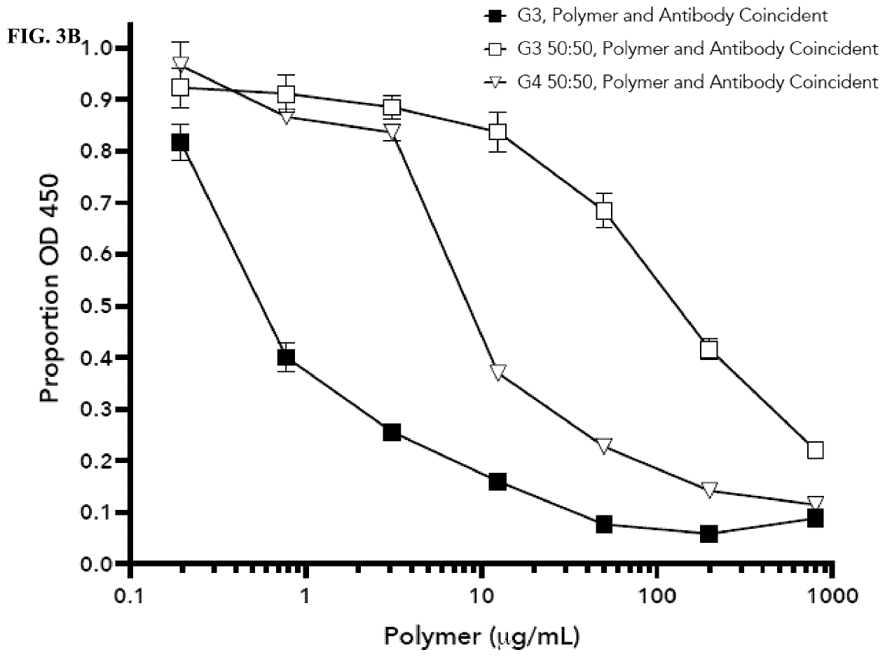

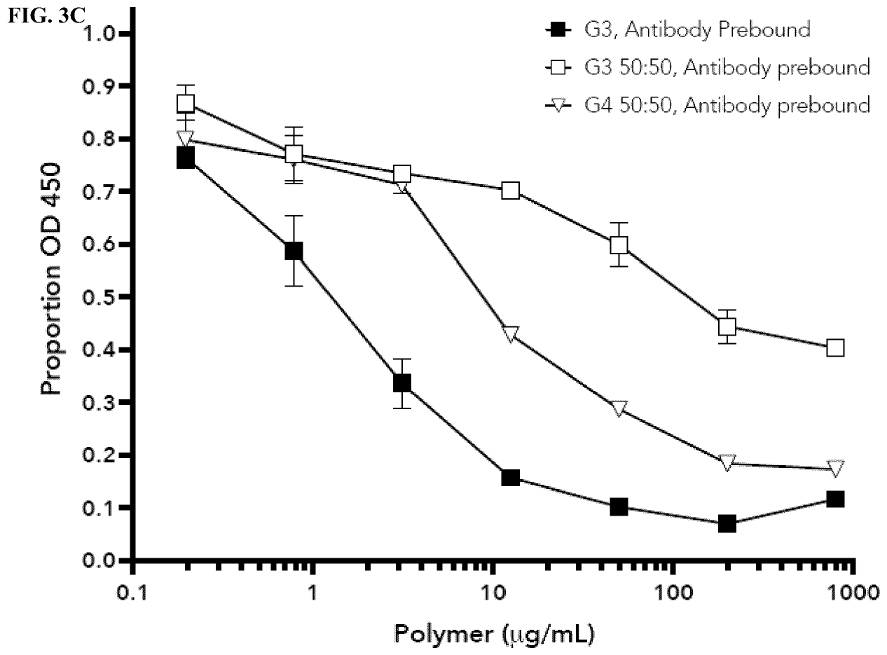

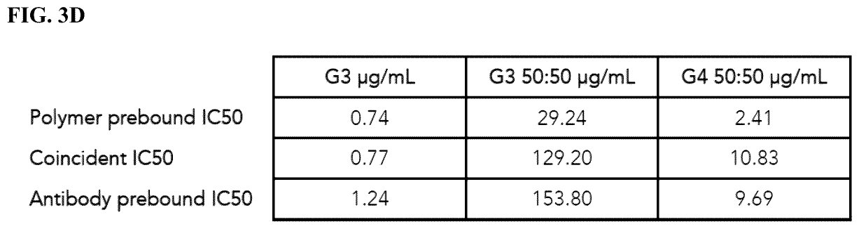

Mixed surface PAMAM variants were then assessed in comparison to PAMAM G3 for their effects on the interaction between DNA and the monoclonal anti-DNA Val-1205 antibody. A uniform amount of ctDNA (100 ng) was allowed to bind to each well. Polymers were then added either prior to the antibody (FIG. 3A), together with the antibody (FIG. 3B), or after the antibody (FIG. 3C). The concentrations at which the respective polymers interfered with antibody binding to DNA by 50% (IC50 values) are shown in FIG. 3D. Relatively low concentrations of PAMAM G3 were capable of limiting association of the antibody with DNA, with 50% antibody binding inhibition occurring with polymer concentrations on the order of 1 μg/mL. In addition, the blocking did not appear sensitive to the relative order of addition of the polymer and the antibody. About 10-fold more of the larger polymer, G4 50:50 was needed for similar inhibition of antibody binding, with less needed if the polymer was added prior to antibody addition. Notably, G3 50:50, with half the overall cationic charge of PAMAM G3, required 100-fold greater concentration than PAMAM-G3 to disrupt antibody-DNA binding and was less effective in competing against prior binding of antibody (FIG. 3C). The reduced ability of G3 50:50 to compete anti-DNA antibody to DNA binding is consistent with our ITC results (FIG. 1).

### Mixed Surface PAMAM Variants Show Significantly Reduced Cytotoxicity Compared to the Fully Cationic Forms

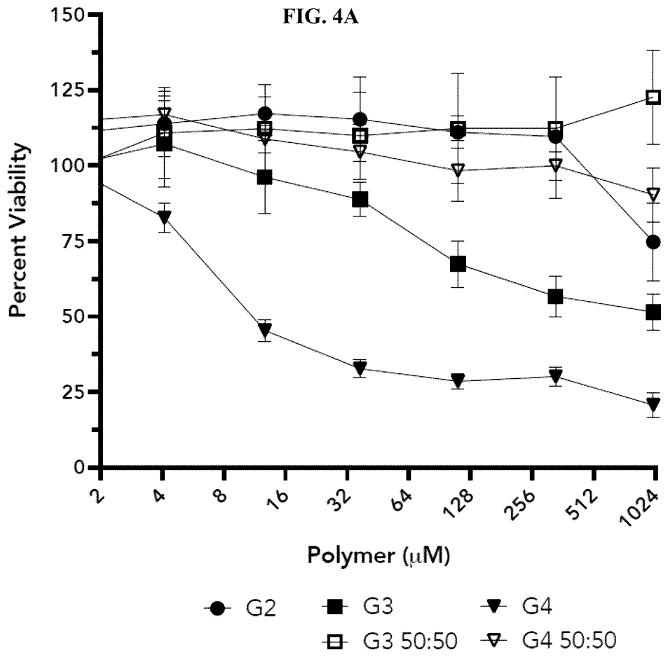

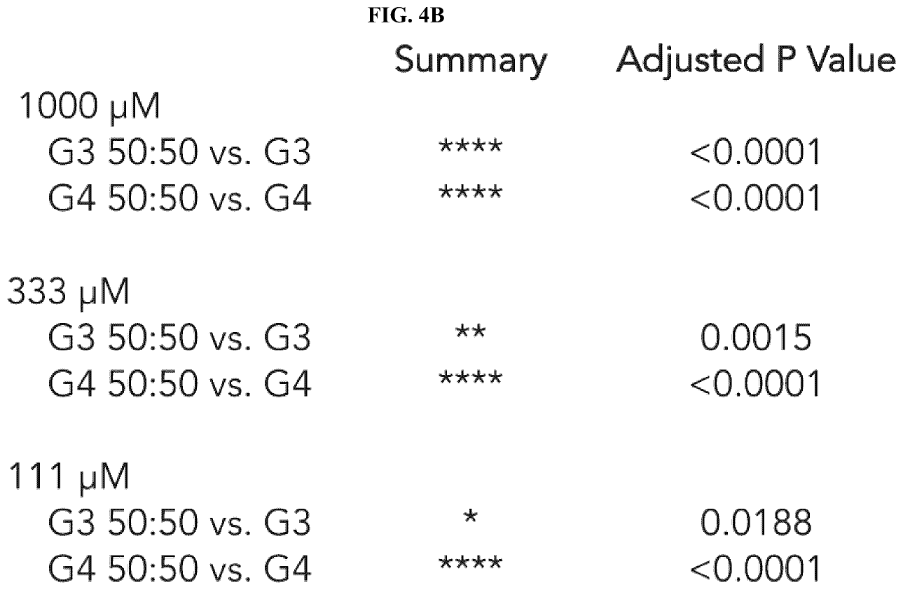

The mixed surface PAMAM variants with more diffuse surface charge were compared to the fully cationic forms for effects on cell viability in culture. HEK293 cells were exposed to the PAMAM variants for 24 hours and relative cytotoxicity assessed by comparing ATP levels using CellTiter-Glo®. Fully cationic PAMAMs G2, G3, and G4 reduced cell viability, with toxicity increasing by generation as previously reported (Malik, Wiwattanapatapee et al. 2000). In contrast, PAMAM G3 50:50 with half its 32 end groups replaced with hydroxyl groups caused no cytotoxicity even at concentrations exceeding 1 mM, while PAMAM G4 50:50, which shares molecular size with G4 but has half of its 64 end groups replaced with hydroxyl groups, caused no cytotoxicity to concentrations of 333 (M and was still less cytotoxic than PAMAM G2 at the highest dose tested (FIG. 4).

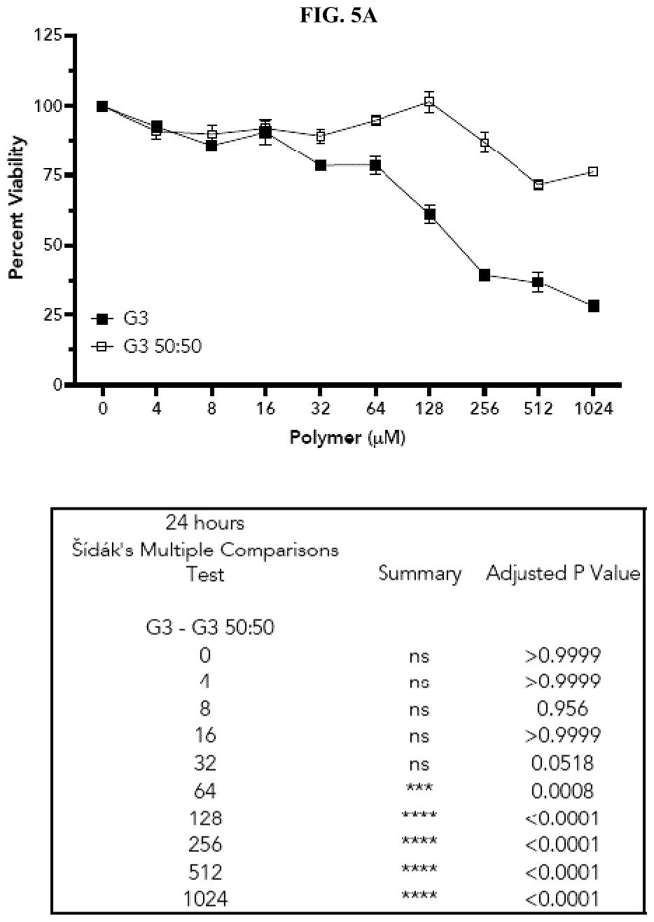

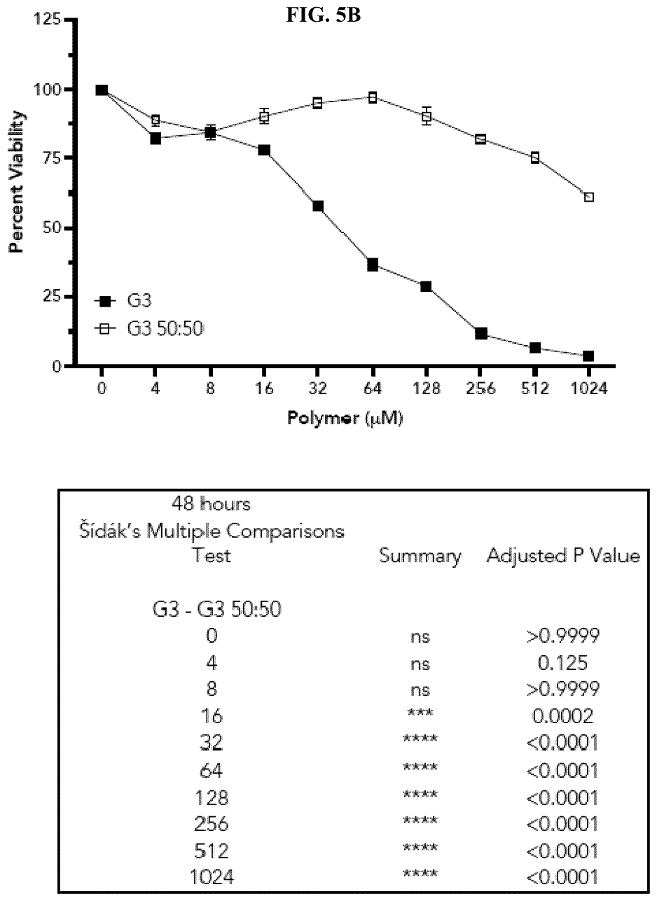

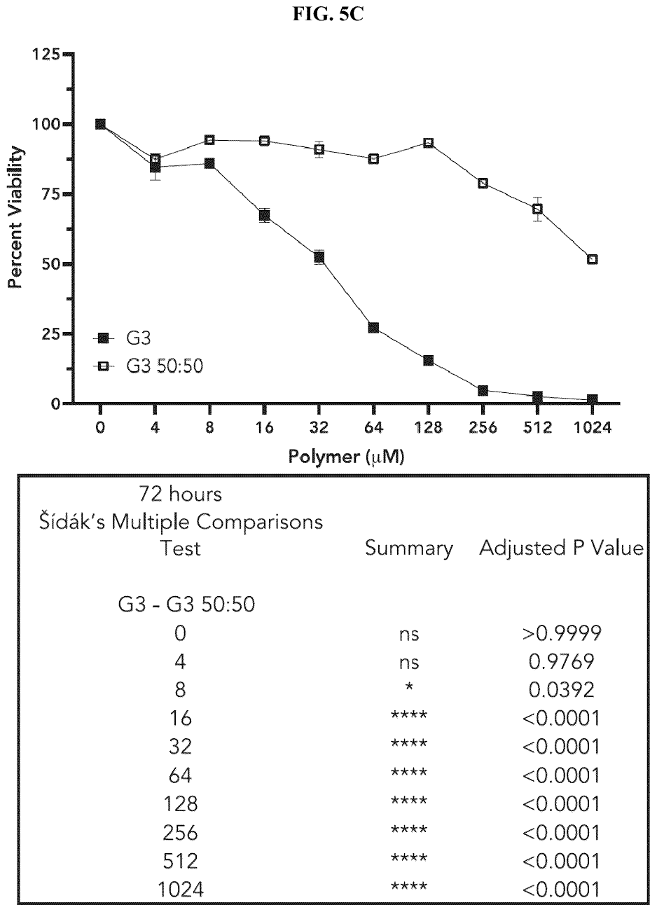

Considering that DAMP signaling is particularly prominent in immune cells and the scavenger benefits likely arise from sustained inhibition of inflammatory signaling, assessment of nucleic acid scavenger effects on the viability of an immune cell line over time is informative. We assessed toxicity using the murine macrophage cell line RAW 264.7. As shown in FIG. 5, from 24 hours (FIG. 5A), to 48 hours (FIG. 5B), to 72 hours (FIG. 5C), PAMAM G3 manifested toxicity at much lower concentrations than G3 50:50. Results from two-way mixed ANOVAs with post hoc analysis via Sidak's multiple comparison tests reveal that G3 50:50 was significantly less toxic than G3 over a wide concentration range. Even after 72 hours, at the highest concentration tested, essentially no cells treated with G3 were viable while 50% of G3 50:50 treated cells remained viable.

### Mixed Surface PAMAM Variants Circumvent In Vivo Toxicity of Cationic PAMAMs

Given the reduced toxicity associated with mixed surface polymers in cell culture, we next examined whether the mixed polymers would be better tolerated in vivo. In three short-term toxicity studies, 20 mg/kg (low dose) and 40 mg/kg (high dose) doses of polymer were delivered to young female C57BL/6J mice. The mice were dosed intraperitoneally every third day for four total doses (days 0, 3, 6, and 9) and sacrificed at day 10. To assess if any polymer effects were transient, an additional set of mice were treated at 40 mg/kg with the same dosing regimen as above, with half sacrificed at day 10 as part of one of the short-term toxicity cohorts and the remaining mice left for an additional 14 days with no further treatment, sacrificed at day 24, and designated as the recovery cohort. The 20 mg/kg and 40 mg/kg doses were chosen as we previously determined that the maximum tolerated dose of PAMAM G3 is 100-200 mg/kg and the 20 mg/kg dose was found to be therapeutic in lupus prone animals (Holl, Shumansky et al. 2016).

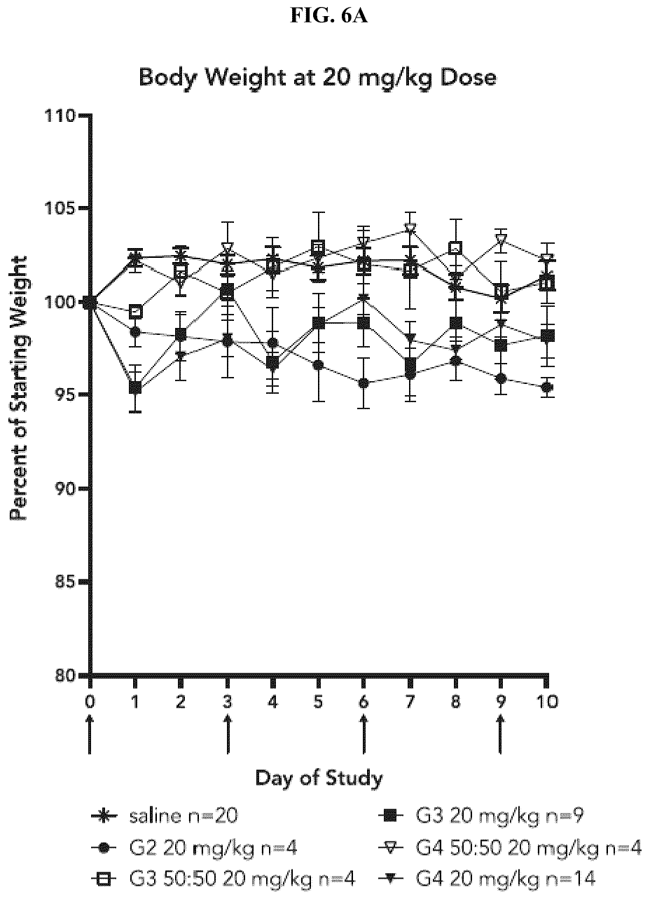

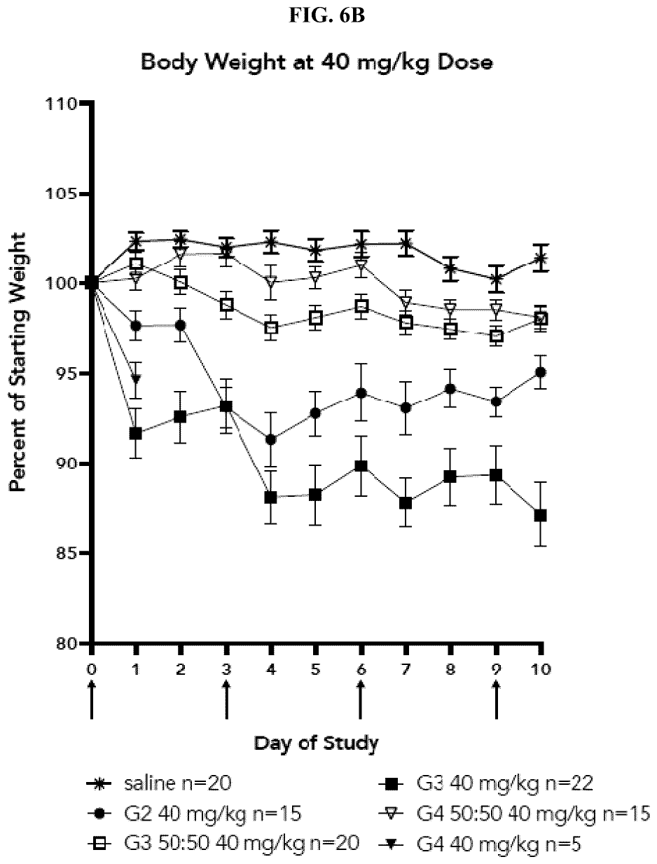

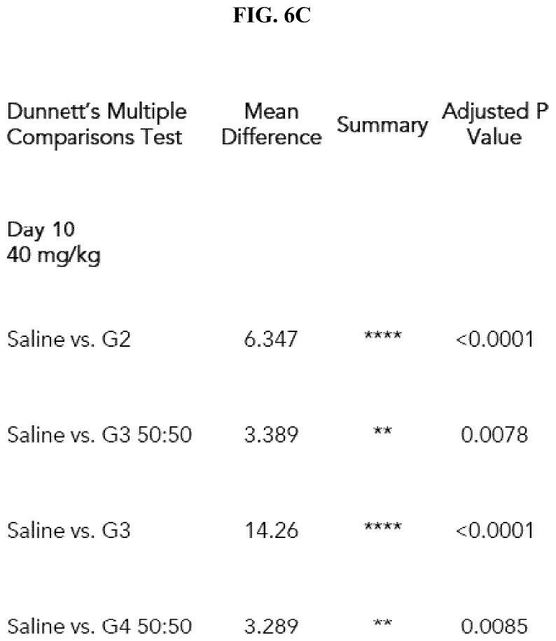

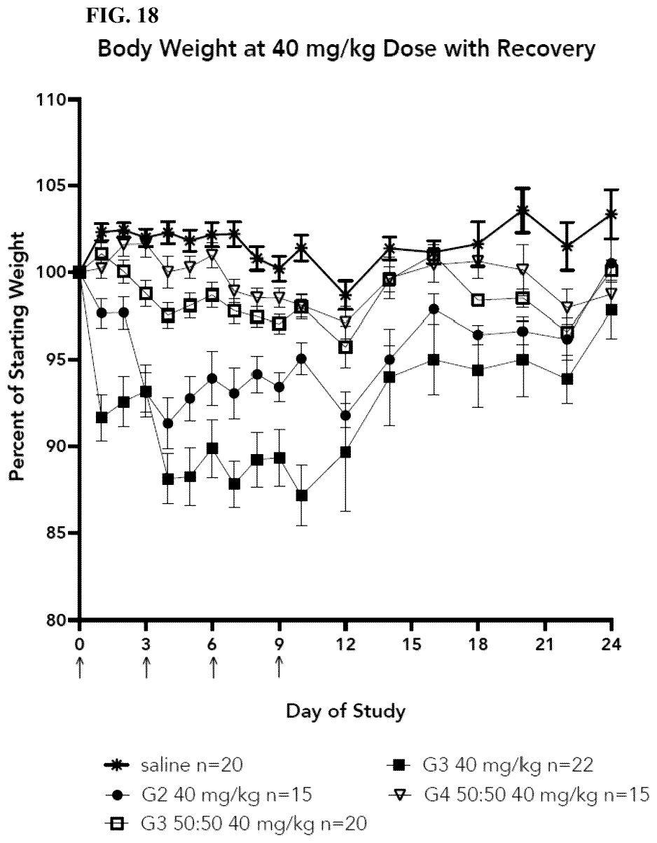

The fully cationic and mixed surface variants were first compared for their effects on mouse body weight. For all mice, weight was measured daily for the first 10 days (FIG. 6) and then for those mice in the recovery cohort every two days from day 10 to day 24 (FIG. 18). The only exception to the planned regimen was the PAMAM G4 40 mg/kg group that was sacrificed 24 hours after the first dose due to acute toxicity. A mixed-effects analysis of weight in the low dose treatment groups (20 mg/kg) was performed to determine the effect of polymer treatment over time. Polymer treatment had a significant effect on weight (P=0.0002). Posthoc comparison of polymer treatment to saline showed significant weight loss on several days for G2 (days 1, 6, 9 and 10), G3 (days 1, 2, 4 and 7) and G4 (days 1-4, 7, 8 and 10) treated mice. As well, PAMAM G3 and G4 treated mice exhibited a telling pattern with an obvious weight dip the day after each dosing and recovery through the next two days. In contrast, the mixed surface polymer treated mice showed no patterned response to dosing and there was very rarely difference in weight between them and saline treated mice (day 1 for G3 50:50 and day 9 for G4 50:50) (FIG. 6A).

In the high dose group (40 mg/kg, FIG. 6B) polymer was found to have a significant impact on weight in mixed-effects analysis (P<0.0001). In posthoc evaluation, G2 and G3 treated mice weighed less than the saline treated mice at all timepoints following treatment on day 0. G4 treated mice also weighed significantly less than the saline treated mice at day 1 but were excluded from the repeated measures analysis because of the acute toxicity which required sacrifice on day 1. G3 50:50 treated animals also weighed significantly less than the saline treated mice at all days but day 1, but the mean difference throughout was smaller than that for the fully cationic polymers at all timepoints, as demonstrated for day 10 (FIG. 6C). G4 50:50 treatment was the mildest in terms of weight loss compared to saline. Cessation of treatment for 14 days prior to sacrifice ameliorated weight loss in mice that had been treated with the PAMAM G2 and G3 at 40 mg/kg (FIG. 18). During this period, the mice in the recovery cohort that had been treated with the mixed surface polymers maintained a relatively stable body weight, while the mice that had been treated with cationic polymers G2 and G3 dramatically rebounded in weight, catching up to the other treatment groups. Thus, the PAMAM G3 50:50 and G4 50:50 variants demonstrate a much-reduced negative impact on animal weight compared to fully cationic PAMAM polymers.

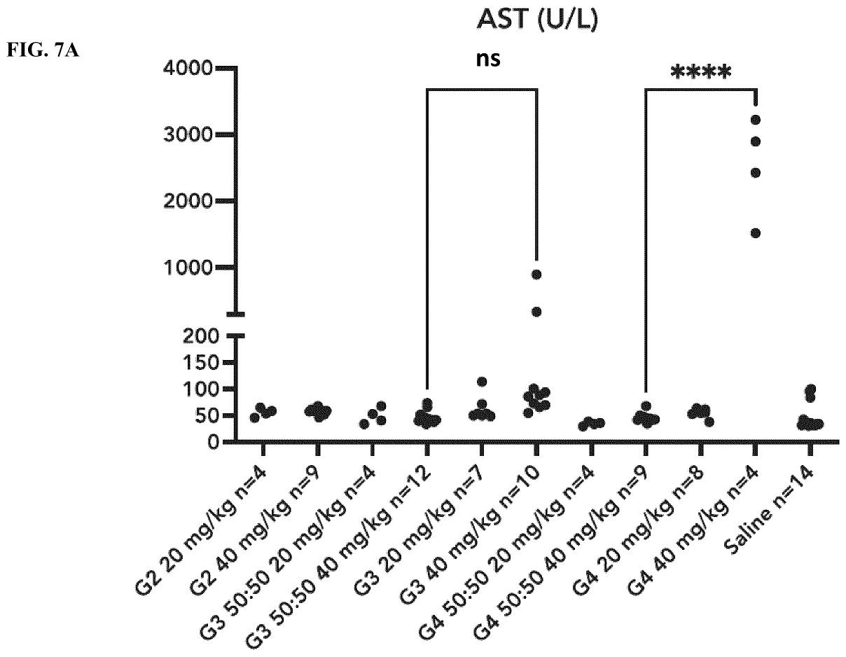

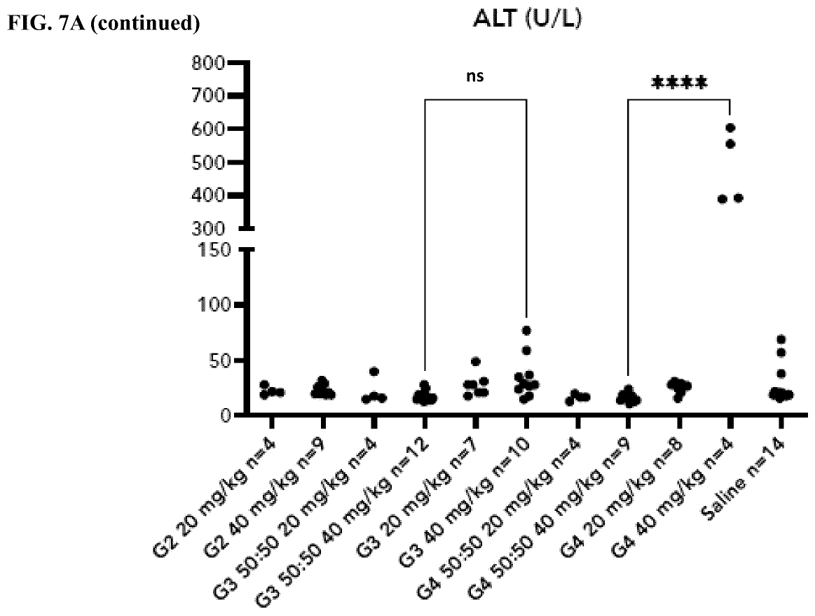

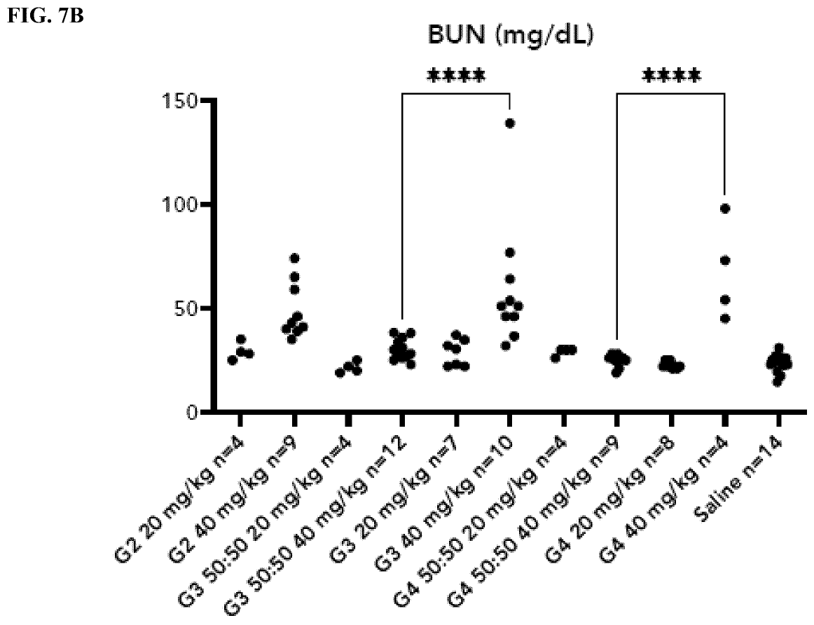

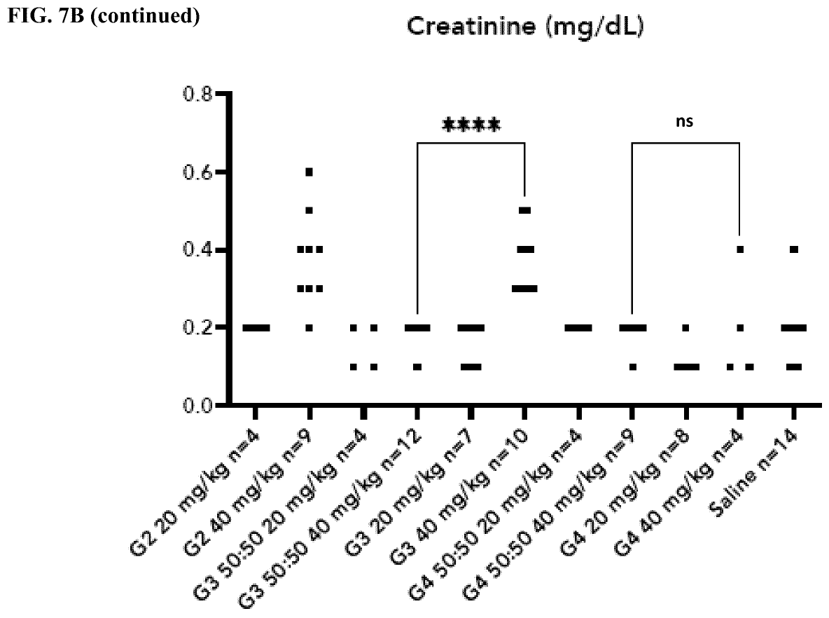

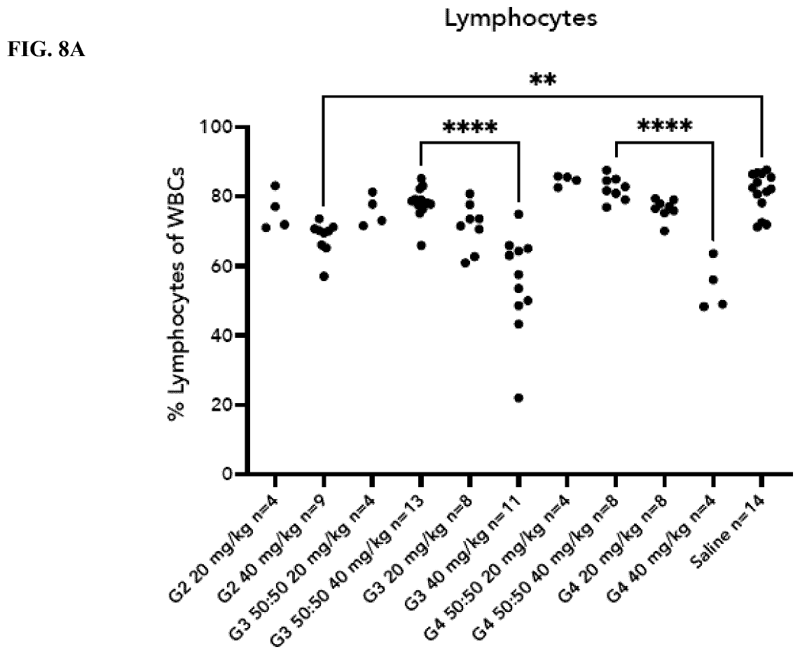

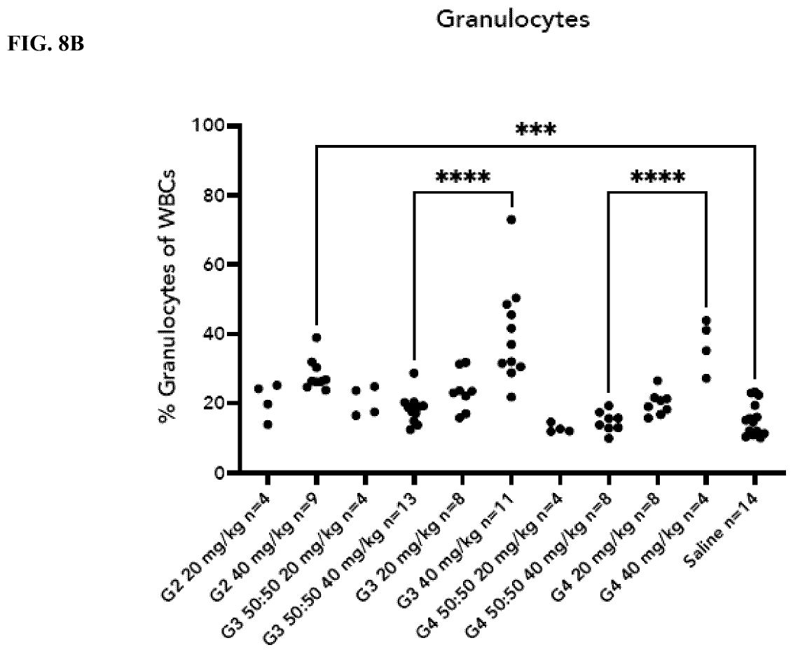

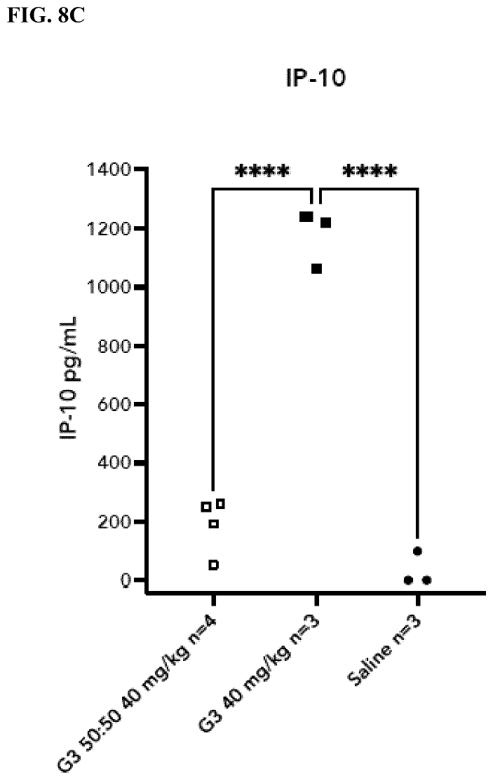

At time of sacrifice for the short-term toxicity experiments, (day 10 for all mice save for G4 at 40 mg/kg, which were sacrificed on day 1 due to acute toxicity), whole blood was collected for complete blood count and serum chemistry analysis, and organs were weighed and fixed. Liver and kidney function tests were performed using mouse serum (FIG. 7). Mice treated with the single 40 mg/kg dose of PAMAM G4 had marked elevations in AST and ALT, indicative of acute liver toxicity. All mice treated with the fully cationic PAMAMs at 40 mg/kg had mild elevations in both BUN and creatinine, indicative of kidney injury. By contrast, treatment with the PAMAM 50:50 variants, even at the higher dose, did not result in notable elevation in any of these liver and kidney toxicity markers. Complete blood count revealed relative lymphopenia (FIG. 8A) and granulocytosis (FIG. 8B) in mice treated with 40 mg/kg of cationic PAMAM G2, G3, and G4, suggesting an acute inflammatory response to treatment with these dendrimers; notably, these effects were much milder in the mice that had been dosed at 20 mg/kg. By contrast, no significant change in lymphocytes and granulocytes was observed in the mixed dendrimer treated animals. Further evaluation of the inflammatory consequences of PAMAM G3 led us to assess serum levels of inflammatory cytokines using multi-analyte flow cytometry. As shown in FIG. 8C, the cytokine IP-10 (CXCL10) was significantly elevated in G3 40 mg/kg treated mice relative to saline treated and G3 50:50 treated mice. IP-10 is a proinflammatory chemokine that causes migration of leukocytes, including monocytes, NK cells, T-cells, and dendritic cells (Dufour, Dziejman et al. 2002). Cytokine IL-6, which broadly stimulates inflammatory and autoimmune processes, and cytokine CXCL1 (KC), a potent neutrophil attractant, also trended higher in the G3 40 mg/kg treated mice; in G3 50:50 40 mg/kg mice they were appreciably lower, with amounts closer to those of saline mice. These cytokine results indicate that the mixed surface polymer G3 50:50 is less proinflammatory than G3 at 40 mg/kg.

Histologic Analysis Reveals that Serosal Inflammation is Triggered by the Fully Cationic Polymer G3 and Reduced in Mice Treated with Mixed Surface Polymers

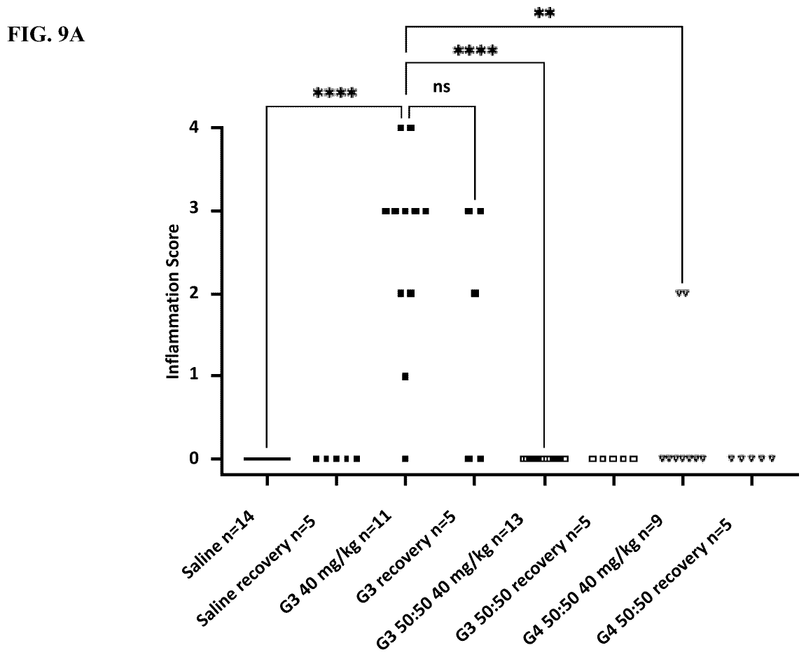

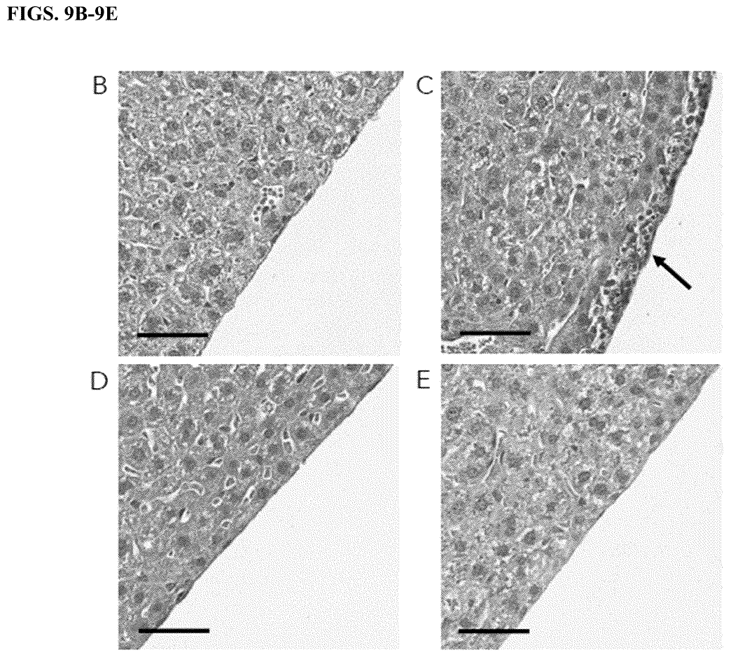

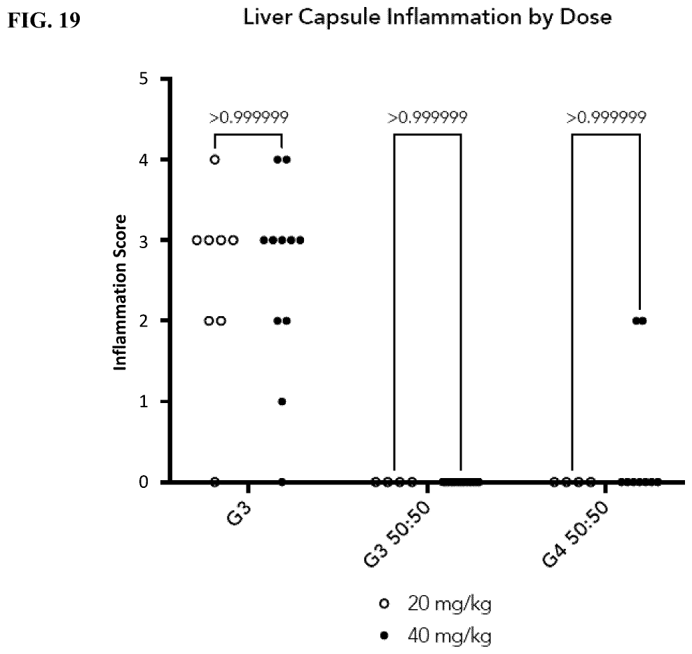

While there were no obvious differences between groups on necropsy or differences in organ weights (data not shown), we carried out histological analysis of the liver, diaphragm and kidneys of mice from G3, G3 50:50, and G4 50:50 treatment groups. Serosal inflammatory changes in mice treated intraperitoneally with fully cationic PAMAM G3 at 20 mg/kg and 40 mg/kg were evident in histological analysis. While PAMAM G3 treated mice exhibited inflammation of the liver capsule, no mice treated with PAMAM G3 50:50 and only two of the nine mice treated with PAMAM G4 50:50 at 40 mg/kg demonstrated this liver surface inflammation consisting of a pleocellular infiltration of neutrophils, macrophages, and lymphoid cells (FIG. 9A and compare 9B-9E). Notably, for the PAMAM G3 recovery cohort the effect was sustained even following the two week recovery period. Liver capsule inflammation was still significant in mice dosed at 20 mg/kg PAMAM G3, in keeping with earlier findings (Kelly, Olson et al. 2022), and shown in FIG. 19. Most obvious on the liver, this capsular inflammation is likely a compound surface property effect from direct contact of the material with cells during the intraperitoneal administration of the compound. Basophilic globules presumed to be dendrimer compound were noted in association with the cellular infiltrate. Other than the surface changes described above, no histologic signs of hepatocytic toxicity were observed in the deeper hepatic parenchyma.

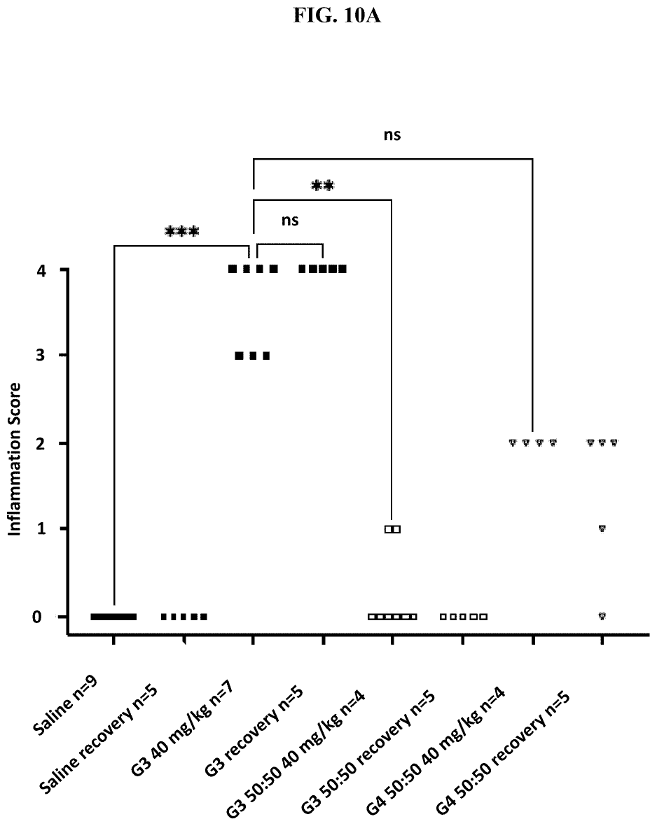

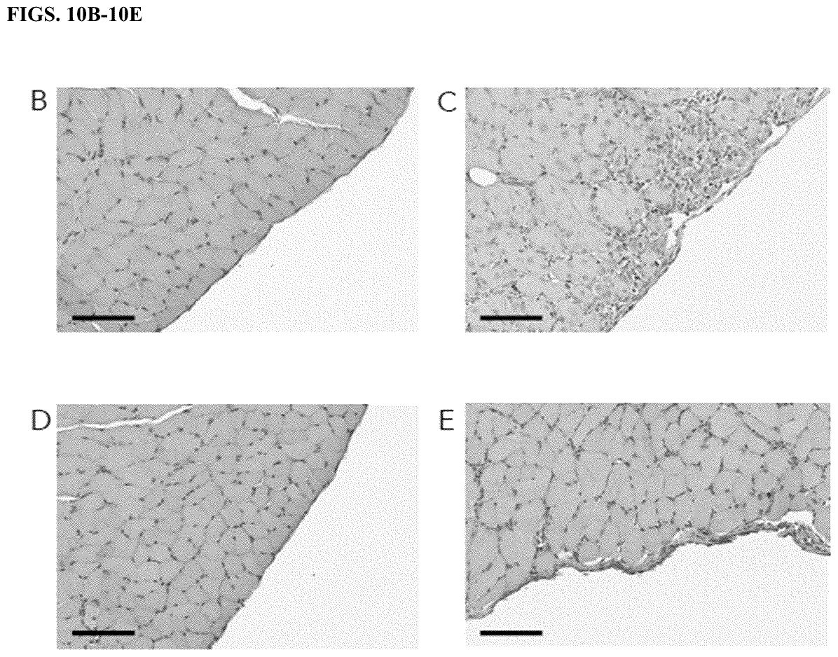

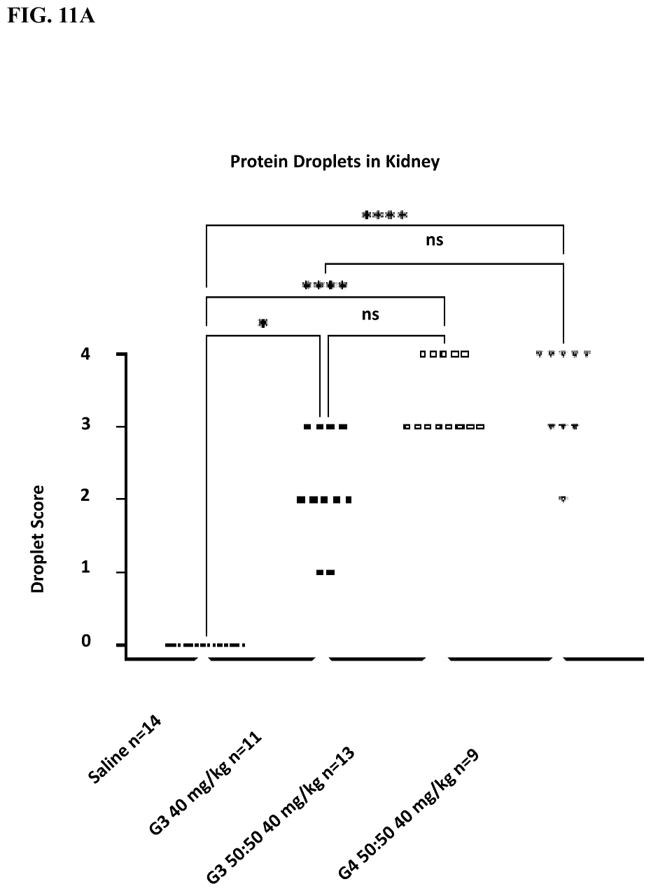

Fortuitous sampling followed by direct study of additional peritoneal organs revealed widespread inflammation of other serosal surfaces upon treatment with PAMAM G3 at 40 mg/kg. The mice treated with fully cationic G3 exhibited severe inflammation of the diaphragm surface and muscle particularly on the abdominal side (FIG. 10) and inflammation of the pancreatic interstitial tissue and peripancreatic fat (data not shown). The diaphragm inflammation seen in G3 treated mice was persistent, with no apparent alleviation in the recovery cohort. The diaphragm surface inflammation presumably is a similar direct contact-induced effect from where compound exits through the diaphragmatic lymphatics following IP injection (data not shown). Very mild inflammation of the diaphragm was seen acutely in some of the G3 50:50 mice at 10 days and was not seen at all in the G3 50:50 recovery cohort at 24 days. Mice treated with G4 50:50 exhibited moderate inflammation of the diaphragm, and occasional inflammation of the pancreas and associated fat as well, evident in both short term and recovery cohort mice. Overall, compared to fully cationic PAMAM G3, there was reduced inflammation with G4 50:50 treatment and almost none with G3 50:50 treatment.

### PAMAM Treatment Induces Kidney Lesions

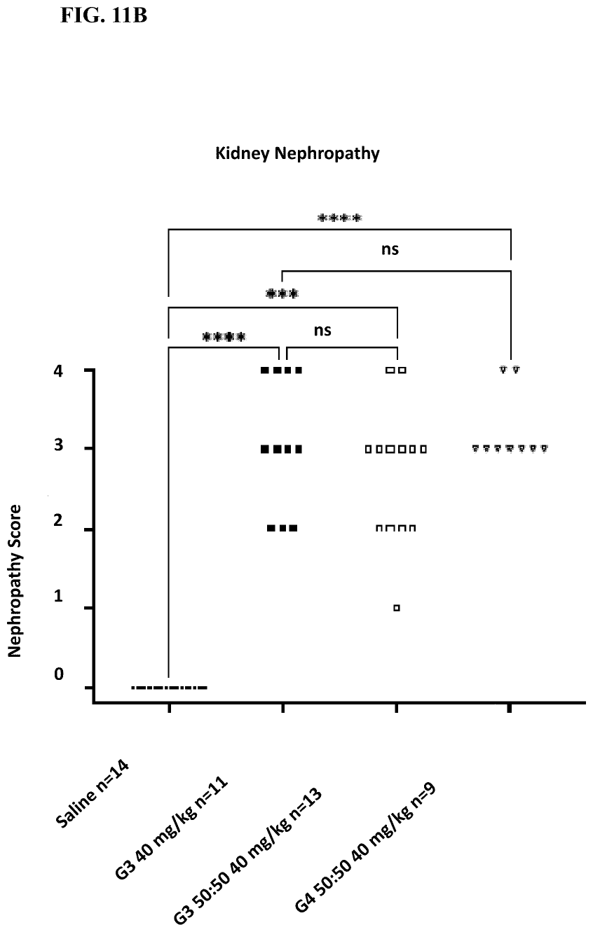

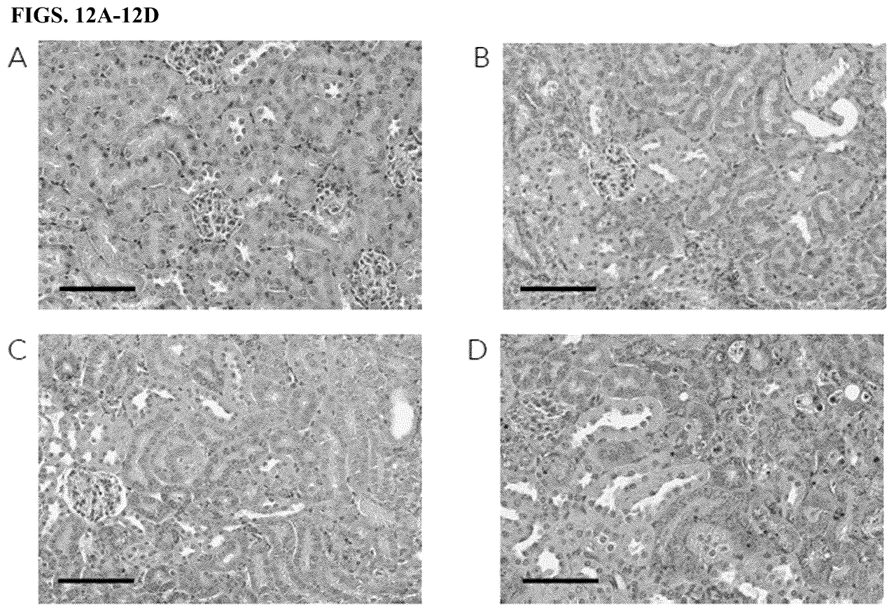

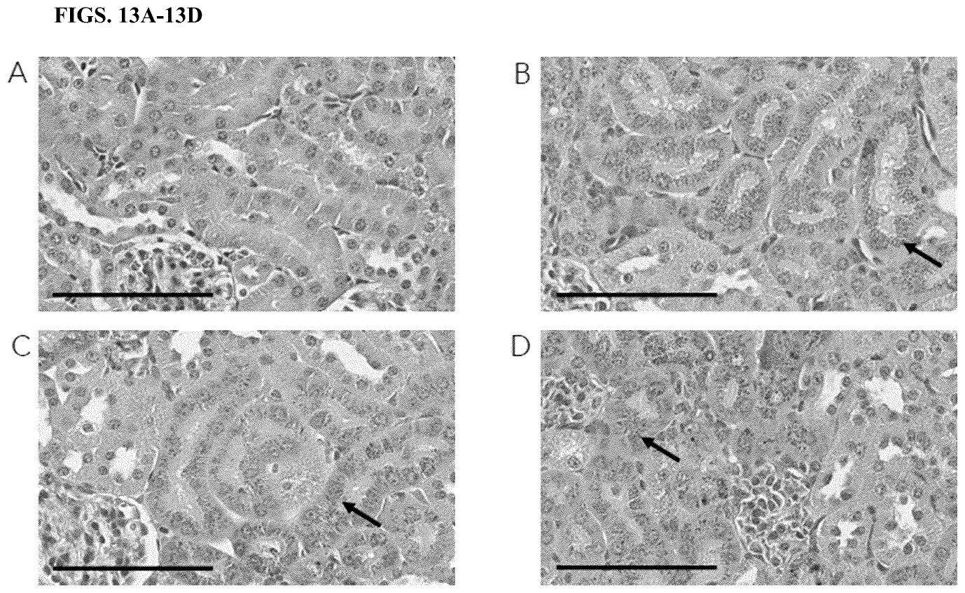

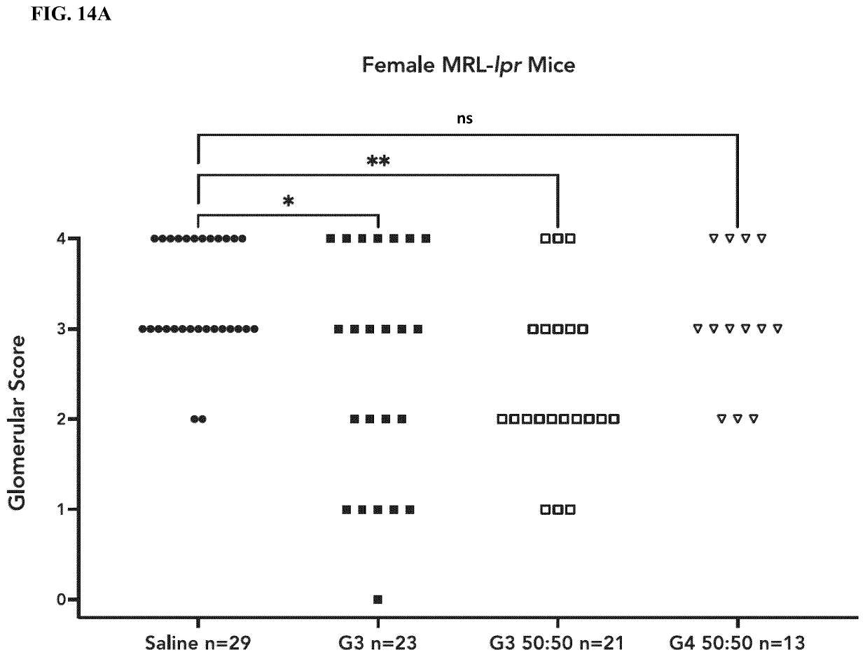

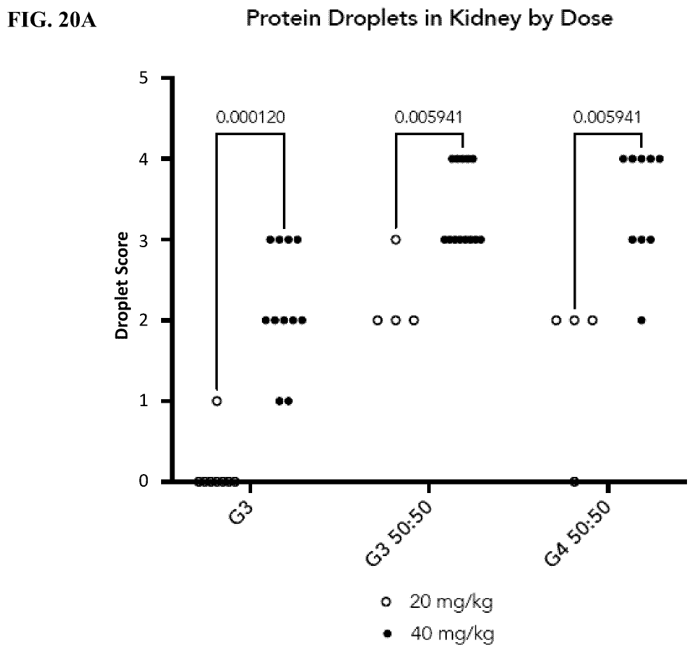

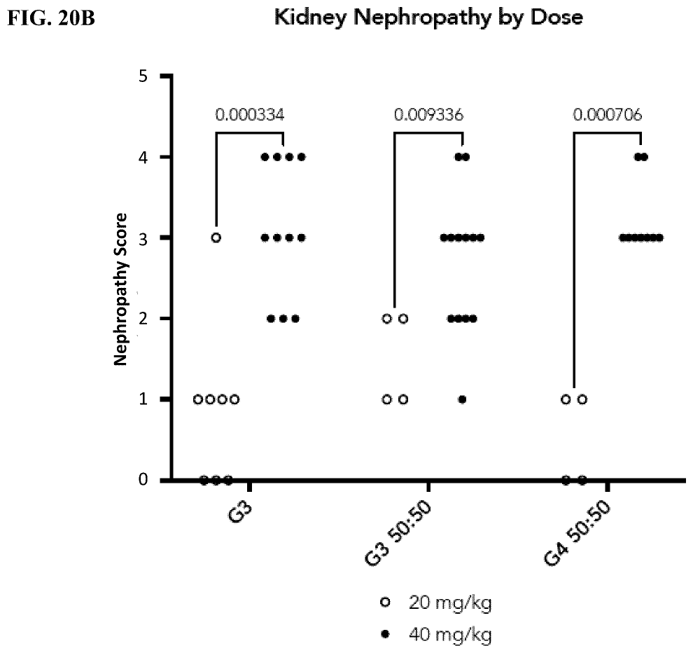

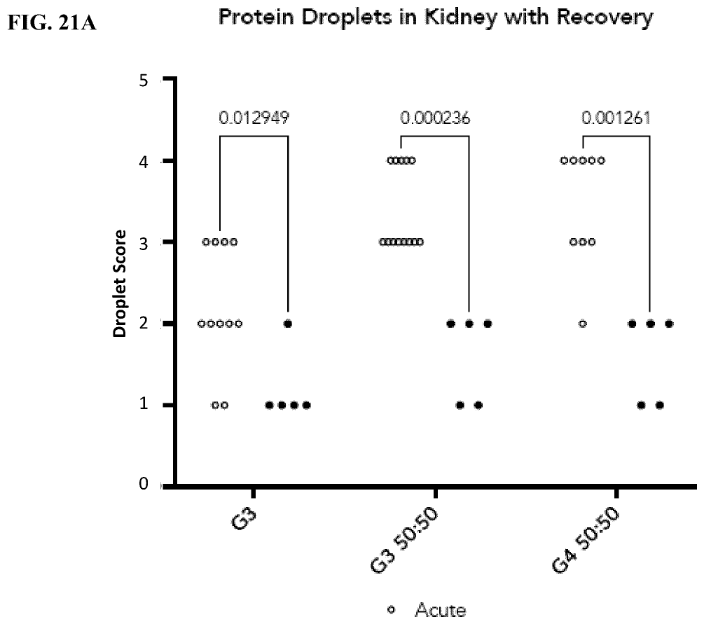

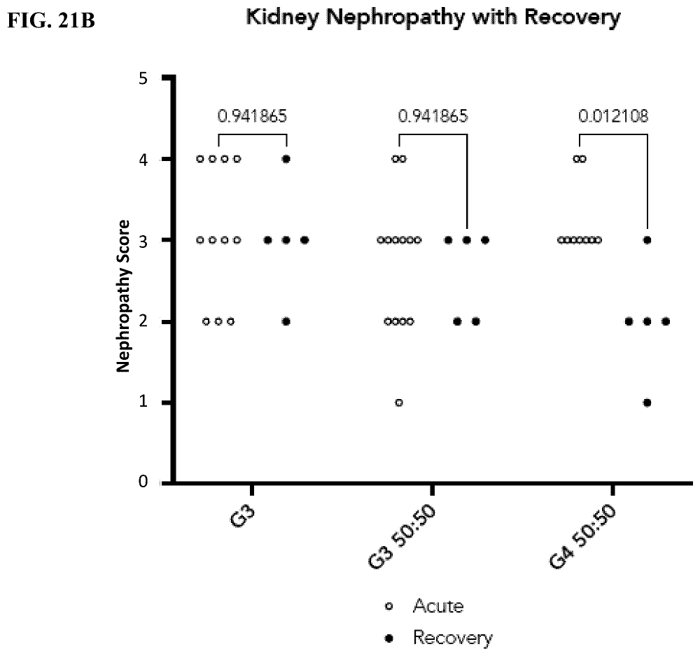

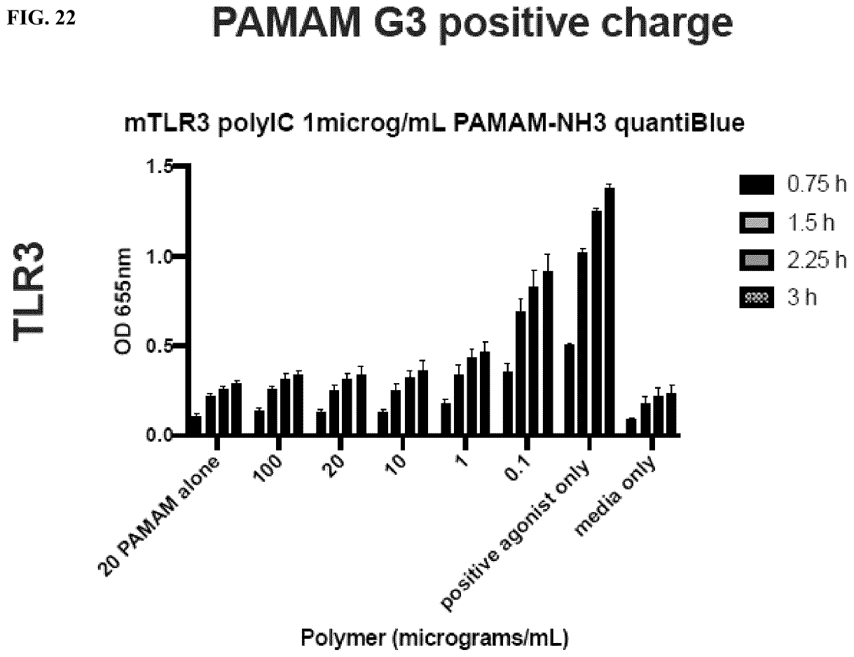

Renal cortical tubular changes were noted in all PAMAM-treated mice, whether treated with fully cationic PAMAM G3 or either of the mixed surface variants. Eosinophilic protein droplet accumulations and nephropathic changes (tubular basophilia and regeneration with hypertrophy and hyperplasia) were observed in treated animals and both tubular lesions were graded semi-quantitatively on a 0-4 scale, with 0 indicating normal kidney and 4 indicating marked change, as described in the methods. The quantitation of these lesions is shown by FIG. 11 and examples of these lesions are shown for all the dendrimer treatment groups in FIGS. 12 and 13. All three PAMAM variants elicit these changes with similar severity in contrast to their disparate effects on kidney function tests. Of note, for all three polymers, the protein droplet accumulation (FIG. 20A) and the nephropathy (FIG. 20B) were significantly less severe in mice dosed at 20 mg/kg (FIG. 20). Protein droplets significantly diminished after cessation of dosing with G3 50:50 and G4 50:50 for two weeks (FIG. 21A). As well, nephropathy scores trended downwards for all three polymers with recovery (FIG. 21B). Of note, small 1 to 5 micron basophilic droplets, believed to be dendrimer compound, are present in all renal sections with lesions graded moderate or greater. Thus, though the inflammatory effects elicited by the fully cationic polymer were reduced when using the mixed PAMAM variants (FIGS. 9 and 10, FIG. 19), the renal depositions characteristic of PAMAM G3 use were not. However, it is unclear what the phenotype if any these alterations may cause given that the mixed polymer treatments did not result in the elevation of kidney damage markers (FIG. 7B).

### Long Term PAMAM G3 50:50 Treatment Reduces Glomerulonephritis in Lupus-Prone MRL-1pr Mice

Following demonstration of the in vitro scavenging competency and overall reduced toxicity in cells and mice, we next assessed if the mixed surface PAMAM variants retained functionality in a mouse model of inflammatory disease. Our group has previously shown that 10 weeks of PAMAM G3 treatment (20 mg/kg twice weekly) beginning at 10 weeks of age reduces glomerulonephritis in MRL-1pr male mice (Holl, Shumansky et al. 2016). The MRL-1pr strain is a model of systemic lupus erythematosus; the mice have a genetic defect in Fas-mediated apoptosis of auto-reactive B and T cells, resulting in high levels of anti-DNA antibodies and spontaneous development of complex-mediated nephritis by 6 months of age. To assess the in vivo DAMP scavenging ability of mixed surface PAMAM variants, female and male MRL-1pr mice were treated twice weekly with 20 mg/kg PAMAM G3, 40 mg/kg G3 50:50, 40 mg/kg G4 50:50, or saline for 9 or 10 weeks beginning at 9 or 10 weeks of age respectively. Higher dosages of the 50:50 molecules were used to match total cationic end groups (G3 50:50) or number of molecules (G4 50:50) with 20 mg/kg cationic G3 treatment. Female mice began treatment and were sacrificed earlier because of the faster progression of lupus-like manifestations in this sex. At 18 or 20 weeks of age, mice were sacrificed and kidneys assessed for severity of glomerulonephritis.

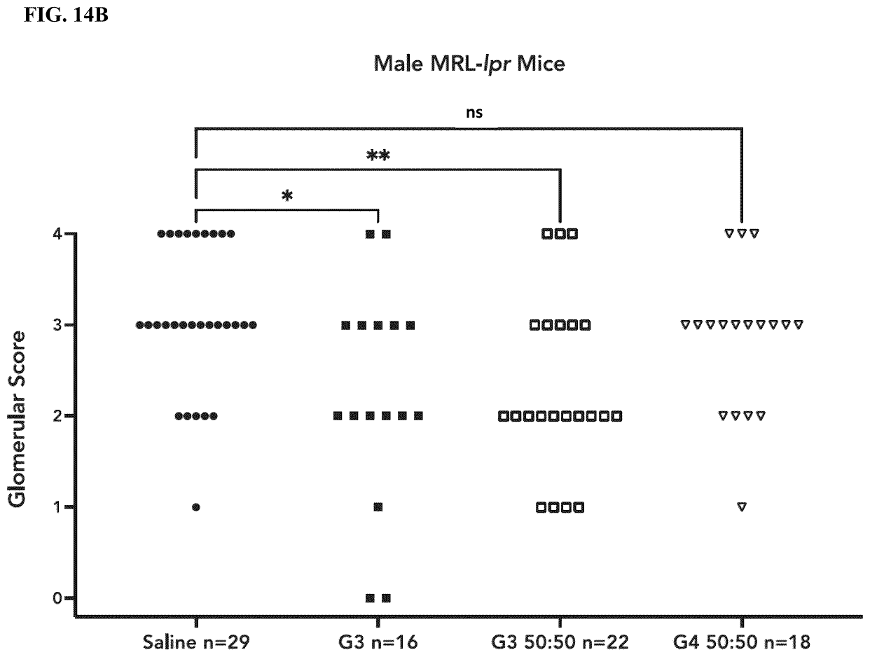

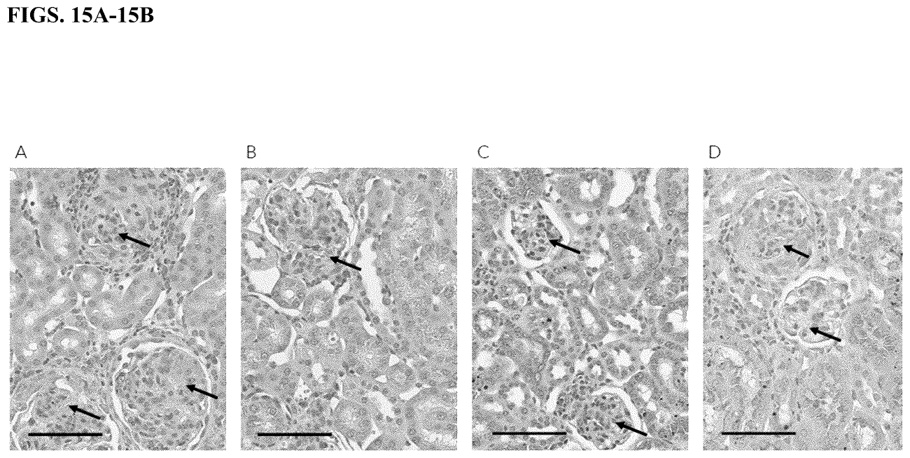

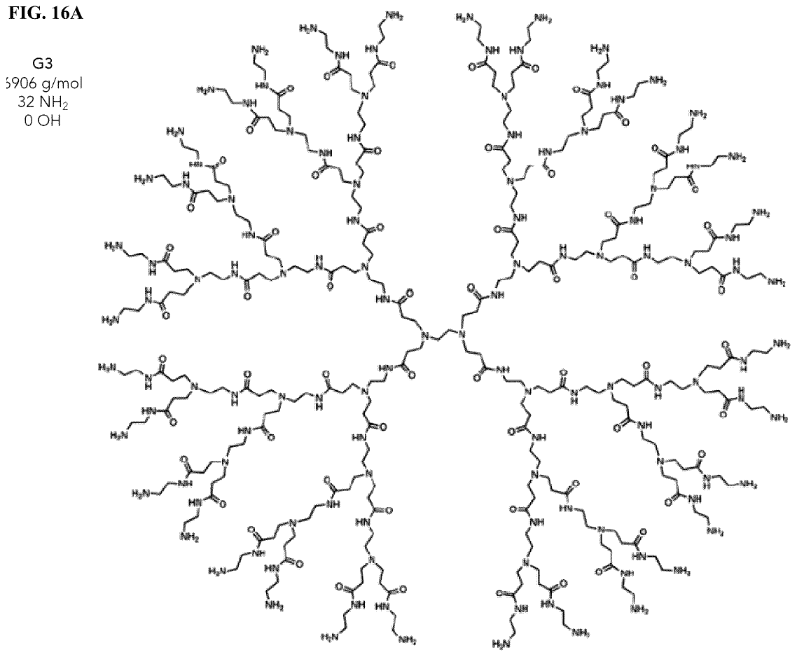

MRL-1pr mice develop a membranoproliferative glomerulonephritis, characterized by glomerular hypercellularity and thickening of the glomerular basement membrane. Lymphoproliferative changes and tubular changes were not scored as part of the lupus assessment. Glomerular change was scored in a masked fashion by a pathologist from 0 (normal), 1 (minimal), 2 (mild), 3 (moderate), through 4 (severe) and results displayed in FIG. 14 (females 14A, males 14B). Application of Kruskal-Wallis non parametric tests revealed that both the female treatment group medians varied significantly (P value=0.0033) and the male treatment group medians varied significantly (P value=0.0101). Post hoc evaluation via Dunn's multiple comparisons test involved the comparison of each polymer to the saline control, to evaluate whether any polymer mitigates the disease evident in the saline-treated mice. In female mice, PAMAM G3 50:50 treatment significantly improved glomerular scores compared to saline treatment (for G3 50:50 adjusted P value 0.0014) while treatment with PAMAM G3 just reached significance (for G3 adjusted P value 0.0441). Similarly in male mice, PAMAM G3 50:50 and PAMAM G3 each improved glomerular scores over saline treatment (for G3 50:50 adjusted P value 0.0100 and for G3 adjusted P value 0.0376). In contrast, the higher generation PAMAM G4 50:50 polymer failed to alleviate glomerulonephritis. Histological preparations of glomeruli of female MRL-1pr mice treated with saline, G3, G3 50:50, and G4 50:50 are shown in FIG. 15.

### Discussion

In this study, we performed experiments to explore whether the toxicity associated with the cationic PAMAM G3 can be reduced while retaining the nucleic-acid scavenging functionality that underpins its anti-inflammatory pharmacologic effects. Specifically, we designed and tested two mixed-surface PAMAM variants, PAMAM G3 50:50 and G4 50:50, which each have the molecular weight near that of their corresponding fully cationic PAMAM of the same generation but have half of their cationic amine end groups replaced with neutral hydroxyl groups. Our results indicate that these mixed surface polymers retain the nucleic acid scavenging capacity and in vivo functionality of PAMAM G3 while avoiding the cellular and most aspects of the in vivo toxicity associated with administration of the fully cationic PAMAMs.

While the generational increase in cationic PAMAM toxicity is well recognized (Roberts, Bhalgat et al. 1996, Malik, Wiwattanapatapee et al. 2000, Jain, Kesharwani et al. 2010, Fox, Richardson et al. 2018), the toxicity of PAMAM generations smaller than four has been less explored. In vivo toxicity data for unmodified, low generation PAMAM dendrimers is even more limited, particularly when the compounds are administered as free molecules not complexed with nucleic acids before injection. Unlike the initial in vivo studies of PAMAM, which suggested that low generation cationic PAMAMs have limited toxicity (Roberts, Bhalgat et al. 1996), our group and others have shown that cationic PAMAM G3 has significant in vivo toxicity, including embryonic toxicity, weight loss, and skin necrosis (Heiden, Dengler et al. 2007, Pryor, Harper et al. 2014, Kelly, Olson et al. 2022). In fact, to evaluate the effects of PAMAM G3 in mice, we first established the LD50 to be 100-200 mg/kg and utilized the compound at a 5-10 fold reduced concentration below this level for efficacy studies (Holl, Shumansky et al. 2016). This study recapitulates these findings by demonstrating generation- and dose-dependent in vitro cytotoxicity and in vivo toxicity in the form of weight loss and abnormalities in liver and kidney function tests at the 20-40 mg/kg dose. Furthermore, our histopathologic findings of substantial intraperitoneal surface inflammation following IP injection of PAMAM G3 suggest that one mechanism of PAMAM's in vivo toxicity is a direct, contact-induced damage to cellular membranes, which worsens with the increasing density of each generation's cationic surface charge. These findings also contextualize the contrasting reports of lethal thrombotic events following intravenous injection of cationic PAMAM G4 in healthy mice but anticoagulant effect of G3 in vascular injury models (Greish, Thiagarajan et al. 2012, Jain, Pitoc et al. 2012). Following injury, the large circulating number of procoagulant DAMPs released from the vascular endothelium, activated platelets or neutrophils are bound and sequestered by the positive surface charges of the scavenger. In the uninjured mouse, those unshielded charges can cause direct toxicity to the vascular endothelium, exposing the procoagulant DAMPs and subendothelium. In this regard, we have observed that diseased animals often tolerate DAMP/PAMP scavenger treatment better than healthy normal control animals just as treatment of cells in culture with cationic polymers bound to DNA or RNA often shows less toxicity than when cells are treated with cationic polymers alone.

This study also provides important biodistribution and excretion data for low generation PAMAMs. While studies of radiolabeled PAMAM have suggested kidney filtration and urinary excretion of smaller generation PAMAM and liver metabolism and distributed visceral organ distribution of generations 4 and larger (Lesniak, Mishra et al. 2013, Labieniec-Watala and Watala 2015), we have not found any other study that examined kidney histology in detail following treatment with low generation PAMAM. We report that treatment with PAMAM G3, or either of the mixed surface dendrimers, G3 50:50 and G4 50:50, results in deposition of protein droplets and nephropathic changes in the renal tubules in normal healthy mice. Interestingly, these changes do not correlate with serum kidney function tests in animals treated with the mixed PAMAM variants even though they do for the fully cationic PAMAMs. The tubular epithelial changes are reminiscent of hydrocarbon nephropathy in rodents where compound binds to proteins in proximal tubules and results in lysosomal overload, cell death and resultant cell proliferation. Although no mechanism can be inferred from the renal lesions in the current study, it would be of interest to examine protein-compound interactions and to see what tubular epithelial organelles are affected. Moreover, it will be important to determine if such protein droplets are less likely to form if high levels of nucleic acid containing DAMPs are present and block such protein interactions with the free polymers.

While the mechanism by which PAMAM G3 50:50 slows progression of glomerulonephritis in murine lupus is not completely defined, previous work has shown that PAMAM G3 can prevent complex formation by sequestering DNA and other negatively charged nuclear antigens, blocking their interactions with autoantibodies in vitro (Stearns, Lee et al. 2012); our studies herein reveal that PAMAM G3 50:50 can also capture DNA and compete with anti-DNA antibody binding albeit less efficiently than the fully modified variant. Since lupus nephritis is driven in part by a TLR-mediated glomerular and tubulointerstitial inflammatory response to renally-deposited immune complexes (Davidson, Berthier et al. 2013), nucleic acid-DAMP scavengers may also modulate complex-driven inflammation in the kidney itself. In this study, we found that the mixed surface PAMAM G3 50:50 scavenges nucleic acid-based TLR agonists comparably to fully modified PAMAM variants (FIG. 1) and significantly reduces glomerulonephritis (FIGS. 14 and 15).

In summary, we report that mixed-surface PAMAM variants are able to bind and neutralize pro-inflammatory nucleic acid-DAMPs in TLR activation assays equivalently to purely cationic polymers with the same number of positive charges with markedly reduced cellular toxicity. In vivo, these mixed-surface polymer variants induced less weight loss and resolved the serosal inflammatory organ injury seen with use of purely cationic PAMAM G3. In addition, the mixed surface polymer G3 50:50 relieved glomerulonephritis in lupus-prone MRL-1pr mice as well or better than fully cationic G3. This study also presents important in vivo toxicology data for the low generation cationic PAMAMs, which are broadly considered to be non-toxic and biocompatible even when administered alone and not as a complex with a nucleic acid. Collectively, these data reveal that mixed surface PAMAMs represent a new class of nucleic acid scavengers with inhibitory activity and improved biocompatibility. Thus the translational development of anti-inflammatory dendrimers is increasingly feasible for lupus and other indications where nucleic acid-DAMP scavenging has shown benefit including autoimmune diseases such as psoriasis (Liang, Yan et al. 2020, Yan, Liang et al. 2021), rheumatoid arthritis (Liang, Peng et al. 2018, Peng, Liang et al. 2019), and inflammatory bowel disease (Shi, Dawulieti et al. 2022), as well as infectious diseases, such as influenza, sepsis (Dawulieti, Sun et al. 2020, Liu, Sheng et al. 2021) and COVID-19 (Naqvi, Giroux et al. 2022), and cancer (Naqvi, Gunaratne et al. 2018, Eteshola, Landa et al. 2021, Holl, Frazier et al. 2021).

Notably, the variant PAMAM G3 50:50, similar in size as PAMAM G3 but with half the charge, was not toxic in cell culture, less associated with weight loss or serosal inflammation after parenteral administration, and remained effective in reducing glomerulonephritis in lupus-prone mice. Identification of such modified scavengers should facilitate their development as safe and effective anti-inflammatory agents.

### Materials and Methods:

### Materials Availability

Two mixed surface PAMAM variants were synthesized as a custom order for us by Dendritech, Inc.: G3 50:50 (16 terminal amines, 16 terminal hydroxyls, ~6.9 kD), and G4 50:50 (32 terminal amines, 32 terminal hydroxyls, ~14.2 kD).

### Experimental Model and Subject Details

### Animal Studies

### Short Term and Recovery Toxicity Studies

Female C57BL/6J mice (RRID: IMSR_JAX: 000664) were obtained from The Jackson Laboratory. Mice were purchased at eight weeks of age and allocated to cages at a density of four to five mice per cage. At ten weeks of age mice were ear punched, weighed, and treatment regimens initiated. At all times, mice received food and water ad libitum. Studies were conducted in accordance with the Guide for the Care and Use of Laboratory Animals and approved by the Duke University Institutional Animal Care and Use Committee (Protocols A201-18-08 and A156-21-07).

### SLE Long Term Treatment Study

Male and female MRL-1pr mice (RRID: IMSR_JAX: 000485) were obtained from The Jackson Laboratory to establish a breeding colony. Mice of this strain are homozygous for the lymphoproliferation spontaneous mutation Faslpr. Purchased female breeder mice produced up to two litters before debilitation from lupus, upon which they were euthanized. Purchased male breeders had had somewhat later onset of lupus, and were euthanized upon debilitation. The MRL-1pr progeny were weaned at three weeks, males and females separated, housed at a density of four mice per cage with random assignment of littermates, and aged. Upon reaching the age of nine weeks for females and ten weeks for males, the mice were ear punched, weighed, and treatment regimens initiated. At all times, mice received food and water ad libitum. The study was conducted in accordance with the Guide for the Care and Use of Laboratory Animals and approved by the Duke University Institutional Animal Care and Use Committee (Protocols A201-18-08 and A156-21-07).

### Cell Lines

### TLR Activation Assays and Cell Toxicity Assays

HEK-Blue hTLR 3, 4, 7, and 9 cells (RRID: CVCL_IM81, CVCL_IM82, CVCL_IM84, CVCL_IM86) derived from the human female HEK293 line were purchased from InvivoGen and maintained in culture according to manufacturer's instructions using a growth media of DMEM, 4.5 g/l glucose, 2 mM L-glutamine, 10% (v/v) fetal bovine serum, 100 U/ml penicillin, 100 μg/ml streptomycin, 100 μg/ml Normocin™. The RAW 264.7 macrophage cell line (RRID: CVCL_0493), derived from ascites induced by infection with the Abelson murine leukemia virus of a male BALB/c mouse, was purchased from the American Type Culture Collection through the Duke CCF Cell Lines Core. RAW 264.7 cells were maintained in culture according to collection's instructions using a growth media of DMEM with 4.5 g/L glucose, 2 mM L-glutamine, sodium pyruvate, and sodium bicarbonate (Sigma D6429-500 mL) supplemented with 10% FBS (not heat inactivated). All cells were grown at 37° C. in 5% CO₂ at 95% relative humidity.

### Method Details

### Polymer Design, Production, and Preparation

Three whole generation cationic polyamidoamine polymers, abbreviated as PAMAMs, were acquired from Dendritech, a commercial manufacturer and supplier of this dendrimer family: G2 (16 terminal amines, ~3.3 kD), G3 (32 terminal amines, ~6.9 kD, and G4 (64 terminal amines, ~14.2 kD). These dendrimers are made by repetitive reactions of ethylenediamine and methyl acrylate to build an organized, highly uniform polymer with low polydispersity and a large number of terminal groups on the dendrimer surface. Two mixed surface PAMAM variants were synthesized as a custom order for us by Dendritech, Inc.: G3 50:50 (16 terminal amines, 16 terminal hydroxyls, ~6.9 kD), and G4 50:50 (32 terminal hydroxyls, ~14.2 kD). In contrast to the usual terminal primary amine surfaces, mixed surfaces can be made that incorporate monoethanolamine in the process, resulting in both primary amidoamine (—NH2) and amidoalcohol (—OH) surface groups. These mixed surfaces can be produced in any desired ratio to tailor the performance properties of the dendrimers for a particular application. The functional groups are statistically distributed across the dendrimer surface, hence the descriptor “mixed” surfaces. Polymers were characterized by 13C NMR to confirm average distribution value.

Polymers were kept lyophilized at −80° C. for long term storage. In preparation for use they were resuspended to 100 mg/mL in sterile dH2O and stored at 4° protected from light. On each day of mouse treatment, polymers were dispersed into 3 mg/mL or 6 mg/mL working solutions with sterile physiological saline and filtered through a 0.2 micron RC membrane filter (VWR Avantor cat #: 28200-024).

### TLR Activation Assays

HEK-Blue hTLR 3, 4, and 9 cells were plated for activation assays in 96-well plates at 40,000 cells per well and allowed to settle overnight. Canonic agonists, high molecular weight Poly(I:C) (1 μg/mL), CpG 1668 oligonucleotide (1 μM), and lipopolysaccharide (LPS-B5, 1 ng/mL) for hTLR 3, 9 and 4 respectively, were purchased from InvivoGen and resuspended according to manufacturer's instructions. Media were then removed and fresh media including control agonists and varying concentrations of polymer in 100 μL total volume were added and allowed to incubate for 24 hours. With no polymer addition the pH of the DMEM medium was pH 7.3. As concentrations of G3, G3 50:50 and G4 50:50 rose, the associated pHs rose as well, but essentially in parallel. For instance, at the 16 μM concentrations the associated pHs were close to 7.6 and at the 32 μM concentrations the associated pHs remained similar to each other at around pH 7.7. After incubation, 40 μL of supernatant was removed from each well to a fresh 96 well plate. Quantiblue (InvivoGen) was resuspended according to manufacturer's instructions, 160 μL was added to wells and incubated for 3 hours at 37° C. The absorbance of the wells was read at 655 nM on a Spectramax i3 (Molecular Devices) plate reader and data presented as percent of maximum activation with canonic agonist alone with each data point the mean of three technical replicates.

### Isothermal Titration Calorimetry (ITC)

Isothermal titration calorimetry for assessing polymer and DNA interactions was conducted using the Nano ITC calorimeter and associated program ITCRun from TA Instruments. Reagents were prepared in PBS without calcium chloride and magnesium chloride (Sigma cat #: D8537). The reference cell was filled with 1 mL PBS. The nucleic acid was the CpG ODN 1668 (InvivoGen cat #: tlrl-1668) and, as outlined above, the polymers were sourced from Dendritech. One milliliter of nucleic acid in PBS (solution pH 7.2) was loaded into the sample cell and polymer in PBS (solution pH 7.3) was titrated into the cell at 10 μL per injection at a stirring rate of 350 rpm for a total of 25 injections at room temperature (25° C.). Spacing between injections was set to 240 seconds for the solution to reach equilibrium. Titrations of each polymer into the sample cell filled with plain PBS were performed to determine the background heat of dilution for the polymers. For the assessment of PAMAM G3 binding to DNA, the polymer was at 37.5 μM in the injection syringe and the oligo was at 13.158 μM in the sample cell. For the assessment of PAMAM G3 50:50 binding to DNA, the polymer was at 62.5 μM in the injection syringe and the oligo was at 13.158 μM in the sample cell. For the assessment of PAMAM G4 50:50 binding to DNA, the polymer was at 37.5 M in the injection syringe and the oligo was at 10.526 μM in the sample cell. calorimetry results were fitted using the TA Instruments provided program NanoAnalyze. For each analysis, the first injection value was omitted and background was removed by applying a linear model generated by the titration of each polymer to the PBS buffer. The thermograms were then fitted using a multiple site binding model constrained to two-sites.

### Enzyme-Linked Immunosorbent Assays (ELISAs)

All ELISA experiments used Immulon 2HB (high binding) flat-bottom, 96 well microtiter plates (Thermofisher 3455, VWR cat #62402-972). Typically, samples had a final volume of 100 μL/well with incubations at room temperature for 1 hour. Washes used phosphate buffered saline (PBS) free of cations at room temperature. Blocking used 200 μL/well of 2% bovine serum albumin, 0.05% Tween-20 in PBS for 2 hours. Calf thymus double stranded DNA (ctDNA) was sourced from Worthington Biochemical Corporation (cat #LS002105), resuspended in TE buffer (Tris 10 mM, EDTA 1 mM, pH 8.0), and extracted several times with phenol: chloroform: isoamyl alcohol (25:24:1; Millipore Sigma cat #77617) and chloroform: isoamyl alcohol (24:1 Millipore Sigma cat #25666). ctDNA was diluted in 1×SSC (150 mM NaCl, 15 mM Na citrate, pH 7). The anti-DNA antibody Val-1205 is a humanized and further modified version of the lupus mouse monoclonal 3E10 developed by Valerion Therapeutics (stock number VALE-915) and kindly provided by Drs. Robert Shaffer and Dustin Armstrong to co-author Dr. David Pisetsky. This primary antibody was typically used at 200 ng/ml and the secondary antibody linked to horseradish peroxidase was used at 1:2000 [anti-human IgG, gamma chain specific, conjugated to peroxidase (Millipore Sigma cat #A6029)]. As needed, antibodies were diluted in 0.1% bovine serum albumin, 0.05% Tween-20 in PBS. Peroxidase activity was assessed by incubation with the peroxidase substrate 3,3′,5,5′-Tetramethylbenzidine dihydrochloride or TMB (Millipore Sigma cat #860336) for 30 minutes, and the reaction stopped by the addition of 2 M sulfuric acid. Absorbance of each well was read at 450 nm using a Molecular Devices Spectra Max i3 plate reader and associated SoftMaxPro 6.3 software.

### Polymer Capture ELISAs

The ability of polymers to bind DNA was assayed by a modified sandwich ELISA composed of plate bound polymer incubated with a dilution series of ctDNA followed by the anti-DNA antibody Val-1205. Polymers were prepared in PBS at 1 μg/mL (solution pH 7.1), directly bound to microtiter plates, incubated with calf thymus DNA of various dilutions (ranging from 0.003 ng/mL to 1000 ng/mL), and the efficiency of DNA capture gauged by the signal arising from binding of the anti-DNA Val-1205 antibody (200 ng/ml).

### Polymer Blocking, Competition and Displacement ELISAs

The ability of polymers to compete with the anti-DNA antibody Val-1205 for the binding of DNA was assayed by ELISA. Microtiter plates were incubated overnight with calf thymus DNA at 5 μg/mL in 1×SSC. The polymers were prepared in ELISA dilution buffer and associated pHs modestly rose with polymer concentration but remained similar to one another: for instance, at 12.5 μg/mL the solution pHs were 7.2, and at 200 μg/mL the solution pHs were 7.5. For the blocking assay a polymer dilution series from 1600 μg/mL to 0 μg/mL was incubated with the DNA for 1 hour prior to addition of the Val-1205 antibody (400 ng/mL, 200 ng/ml final), incubated jointly for 1 hour, washed and then incubated with the secondary antibody. For the competition assay the same polymer dilution series as above was added together with the Val-1205 antibody (400 ng/mL, 200 ng/mL final) for an incubation period of 1 hour, washed and then incubated with the secondary antibody. For the displacement assay, the Val-1205 antibody (400 ng/mL, 200 ng/ml final) was added to the plate for one hour prior to the addition of the polymer dilution series, incubated jointly for 1 hour, washed and then incubated with the secondary antibody.

### Cell Toxicity Assays

HEK-Blue hTLR 3, 4, 7, and 9 cells were plated for toxicity assays in 96-well plates at 40,000 cells per well and allowed to settle overnight at 37° C. in 5% CO₂ at 95% relative humidity. Media were then removed and fresh media with varying concentrations of polymer in 100 μL total volume were added and allowed to incubate. Associated pHs of the culture media rose with polymer concentration, similarly for all the polymers tested: for instance, at 16 μM the solution pHs were 7.6, at 64 μM the solution pHs were 7.9, and at 256 μM the solution pHs were 8.3. The CellTiter-Glo® luminescent cell viability assay was purchased from Promega and prepared according to manufacturer's instructions at time of assay. After 24 hours, 100 μL of CellTiterGlo reagent was added to each well and contents mixed on an orbital shaker for 2 minutes and then incubated at room temperature for 10 minutes. The luminance of the wells was read on a Spectramax i3 (Molecular Devices) plate reader and data presented as percent of luminance for cells alone without added polymer. Data are the combined means of the 4 cell lines tested, with three technical replicates averaged to generate the data point for each cell line.

RAW 264.7 cells were plated for time course toxicity assays in 96-well plates at 7,000 cells per well and allowed to settle overnight at 37° C. in 5% CO₂ at 95% relative humidity. Media were removed and replaced with fresh media with varying concentrations of G3 or G3 50:50 polymers in 100 μL total volume added and allowed to incubate. Media pH increased uniformly as polymer concentrations increased, as described above for the HEK studies. The CellTiter-Glo® luminescent cell viability assay was purchased from Promega and prepared according to manufacturer's instructions at time of assay. At 24, 48, and 72 hours, 60 μL of CellTiterGlo reagent was added to each well and contents mixed on an orbital shaker for 2 minutes and then incubated at room temperature for 10 minutes. The luminance of the wells was read on a Spectramax i3 (Molecular Devices) plate reader. Data are the average of three technical replicates and presented as percent of luminance for cells alone without added polymer.

### General Notes on the Studies in Mice

No power calculation was used to determine group sizes; group sizes were determined by logistical feasibility and experience of authors gained from earlier studies.

### Short Term Toxicity Studies in Mice

Short-term toxicity was assessed over three studies under a repeated protocol but with varied polymers and doses. Female C57BL/6J mice received intraperitoneal injections of saline or 20 or 40 mg/kg of selected dendrimer polymers: PAMAM G2, G3 50:50, G3, G4 50:50, or G4 every three days for 4 total doses. Mice were evaluated and weighed daily and sacrificed 24 hours after final injection. At sacrifice, blood was collected for complete blood count and serum prepared for liver and kidney function tests and quantitation of inflammatory markers. Necropsy was performed at time of death by a veterinary pathologist and organs were weighed and fixed in 10% formalin and processed as FFPE samples for H&E staining. Livers were assessed for hepatic surface inflammation based on pleocellular infiltration of neutrophils, macrophages, and lymphoid cells. Kidneys were scored for protein droplet accumulations and for nephropathic changes on a semi-quantitative score of 0-4 with zero being normal kidney and 4 being the most severe change. Protein droplet scores were based on relative number and size of droplets and number of tubules involved. Nephropathy scores were based on % cortex involved as well as the degree of tubular epithelial change involving basophilia and regenerative hypertrophy and hyperplasia. Scores ranged from 0 (normal), 1 (minimal), 2 (mild), 3 (moderate), through 4 (severe). Serum chemistry tests were performed by IDEXX labs. The first study involved all polymers at both doses (20 mg/kg and 40 mg/kg) and used 4-5 mice per treatment group; the second study, done in conjunction with the “Recovery Study” described below, involved 40 mg/kg dosing of all polymers and used 4 to 5 mice per treatment group; the third study focused on G3 and G3 50:50 polymers and used 5 to 7 mice per treatment group. Numbers of animals in each group do show variability dependent on the particular assay: weight assessment, reflecting the numbers of mice at the start of the treatments, has the highest n; the histological analyses show a modest dropoff in numbers as the occasional animal expired during the study; and serum chemistry numbers are lower because serum was not acquired in all contexts, notably for the recovery mice described below.

Analysis of Inflammatory Cytokines using LEGENDplex

Quantitation of mouse cytokines in serum obtained from mice in the final short term toxicity study described above was accomplished using the LEGENDplex™ Mouse Anti-Virus Response Panel (BioLegend) and the associated LEGENDplex™v8 Data Analysis Software. Each serum sample was diluted 1:1 and then assessed in two wells. The assay and acquiring of the flow samples on a Beckman Coulter CytoFLEX flow cytometer followed the detailed instructions provided by the manufacturer's LEGENDplex™ manual. After data analysis, the rare samples where the cytokine concentration was determined to be below detection were assigned the value of zero.

### Recovery Study in Mice

To assess for transience of polymer-induced inflammation and kidney lesions, the above study's protocol was repeated with G2, G3, G3 50:50 and G4 50:50 dosed at 40 mg/kg, with the mice left untreated after final dosing for two additional weeks before sacrifice. Five mice per group were treated with 4 doses of polymer or saline, and weighed every other day for two weeks following final treatment, and then sacrificed with dissection of the liver, kidneys, and diaphragm from each mouse and processed as FFPE samples for H&E staining.

### SLE Long Term Treatment

Female and male MRL-1pr mice were weighed and received intraperitoneal injections of PAMAM-G3 (20 mg/kg, Nf=23, Nm=16), PAMAM-G3 50:50 (40 mg/kg, Nf=21, Nm=22), PAMAM G4 50:50 (40 mg/kg, Nf=13, Nm=18), or saline (Nf-29, Nm=29) twice per week; in the case of females for a period of 9 weeks starting at 9 weeks of age and in the case of males for a period of 10 weeks starting at 10 weeks of age. Mice were then sacrificed, and citrated plasma and tissue were collected for further analysis. For analysis of kidney disease, kidneys were collected and further processed as FFPE samples for H&E staining. Glomerulonephritis in the MRL-1pr mice was scored based on glomerular hypercellularity and thickening of glomerular basement membranes. Lymphoproliferative changes and tubular changes were not scored as part of the lupus assessment. Glomerular change was scored from 0 (normal), 1 (minimal), 2 (mild), 3 (moderate), through 4 (severe).

### Quantification and Statistical Analysis

Table 2 documents the statististical analyses used in the study. Much of the information as to the test used, n, and error, is also presented in the associated figure legend. For continuous data, one-way ANOVA, two-way ANOVA, mixed-effects models, and IC50 determinations were performed using GraphPad Prism v9.4.1. Significant ANOVAs were followed by Tukey's post hoc test for comparison of all groups to one another and mixed-effects models were followed by Dunnett's post hoc test for comparison of polymer groups to saline treated contols. Analysis of ordinal tissue score data arising from the toxicity studies in C57BL/6J females and from the treatment studies of lupus prone MRL-1pr females and males was guided by Meyerholz et al. (Meyerholz, Tintle et al. 2019), and used GraphPad Prism v9.4.1. The Kruskal-Wallis nonparametric test was applied to determine whether any treatment group median was different from the others. Upon evidence for difference, post hoc evaluations using the Dunn's multiple comparisons test were performed. For the toxicity studies, the comparisons generally compared all groups to one another. However, when comparing particular dosage or washout effects for each particular polymer, multiple Mann-Whitney tests with a false discovery rate set to 1% were employed with q values (false discovery rate adjusted P values) indicated. For the SLE treatment study of MRL-1pr mice, comparisons were made with respect to the control saline mice treatment group. For all tests, *P<0.05, ** P<0.01, *** P<0.001, and *** P<0.0001.

TABLE 2
Statistical summary for paper. Listing of the statistical tests used and associated parameters for the data presented.
Statistics for experiments in Olson et al.

| FIG. or | | Statistical | | | Points on |
|---|---|---|---|---|---|
| Table | Experiment | Method | Comparisons | n | Graph |
| FIG. 1 | TLR inactivation | IC50: log(inhibitor) | | 3 technical | Graph - |
| | | vs. normalized | | replicates | mean |
| | | response [symbol] Variable | | | |
| | | slope, fitting | | | |
| | | method is least | | | |
| | | squares regression | | | |
| Table 1 | Thermodynamic | Nano Analyze [symbol] | | | |
| | parameters | derived | | | |
| | | characteristics | | | |
| FIG. 2 | Capture ELISAs | Ordinary two-way | Within each row (DNA | 4 technical | Graph - |
| | | ANOVA, post hoc | concentration), compare | replicates | mean with |
| | | Tukey's multiple | columns (polymer type) [symbol] | | error bars |
| | | comparisons test | compare each cell mean | | SEM |
| | | | with every other cell | | |
| | | | mean on that row | | |
| FIG. 3 | Comparison | IC50: log(inhibitor) | | 4 technical | Graph - |
| | ELISAs | vs. normalized | | replicates | mean with |
| | | response - Variable | | | error bars |
| | | slope,fitting | | | SEM |
| | | method is least | | | |
| | | squares regression | | | |
| FIG. 4 | Cell [symbol] HEKs | Ordinary two-way | Within each row (viability | each data | Graph - |
| | | ANOVA, post hoc | at particular | point is the | mean with |
| | | Tukey's multiple | concentration), compare | mean of 4 | error bars |
| | | comparisons test | columns (polymer type); | HEK lines | SEM |
| | | | compare each cell mean | (each with 3 | |
| | | | with every other cell | technical | |
| | | | mean on that row | replicates) | |
| FIG. 5 | Cell [symbol] RAW | Ordinary two-way | Within each row (viability | 3 technical | Graph - |
| | 264.7 | ANOVA, post hoc | at particular | replicates | mean with |
| | | Sidak's multiple | concentration), compare | | error bars |
| | | comparisons test | columns (polymer type); | | SEM |
| | | | compare each cell mean | | |
| | | | with every other cell | | |
| | | | mean on that row | | |
| FIG. 6 | Body weight of | Mixed-effects | Each row represents a | n indicated | Graph - |
| panel A | mice low 20 | model, post hoc | different time point | for every | mean with |
| | mg/kg | Dunnett's testing | (repeated measures), so | group | error bars |
| | | for comparison of | matched values are | | SEM |
| | | polymer groups to | stacked into a | | |
| | | saline treated | subcolumn; compare | | |
| | | controls | each cell mean to the | | |
| | | | saline cell mean on that | | |
| | | | row | | |
| FIG. 6 | Body weight of | Mixed-effects | Each row represents a | n indicated | Graph - |
| panel B | mice high 40 | model, post hoc | different time point | for every | mean with |
| | mg/kg | Dunnett's testing | (repeated measures), so | group | error bars |
| | | for comparison of | matched values are | | SEM |
| | | polymer groups to | stacked into a | | |
| | | saline treated | subcolumn [symbol] compare | | |
| | | controls | each cell mean to the | | |
| | | | saline cell mean on that | | |
| | | | row | | |
| FIG. 7 | Serum | Ordinary one-way | Compare the mean of | n indicated | Graph - |
| panels A and | Chemistries | ANOVA, post hoc | each column with the | for every | Individual |
| B | AST, ALT, BUN, | Tukey's multiple | mean of every other | group | animals |
| | Creatinine | comparisons test | column | | shown |
| FIG. 8 | Whole Blood | Ordinary one-way | Compare the mean of | n indicated | Graph - |
| panels A and | lymphocyte and | ANOVA, post hoc | each column with the | for every | Individual |
| B | granulocyte % | Tukeys's multiple | mean of every other | group | animals |
| | of WBCs | comparisons test | column | | shown |
| FIG. 8 | Legendplex, | Ordinary one-way | Compare the mean of | n indicated | Graph - |
| panel C | specifically [symbol] | ANOVA, post hoc | each column with the | for every | Individual |
| | 10 chemokine | Tukey's multiple | mean of every other | group | animals |
| | | comparisons test | column | | shown |
| FIGS. 9A, | Liver, | Kruskal-Wallis test, | Compare the mean rank | n indicated | Graph - |
| 10A, 11A and | diaphragm and | post hoc Dunn's | of each column with the | for every | Individual |
| B | kidney histology | multiple | mean rank of every other | group | animals |
| | scores | comparisons test | column | | shown |
| FIG. 14 | MRL [symbol] kidney | Kruskal-Wallis test, | Compare the mean rank | n indicated | Graph - |
| | histology scores | post hoc Dunn's | of each column with the | for every | Individual |
| | | multiple | mean rank of a control | group | animals |
| | | comparisons test | column - Group A [symbol] Saline | | shown |
| Supplemental | Liver and kidney | Multiple Mann- | Individual ranks | n indicated | Graph - |
| FIGS. 4, 5, | histology scores | Whitney tests with | computed for each | for each | Individual |
| 6 | involving | false discovery rate | comparison (eg, low | group in | animals |
| | comparisons of | set to 1% | dose vs high dose for | figure | shown |
| | particular | | specified polymer) with | legend | |
| | polymer dose | | the pairwise comparison | | |
| | amounts and | | value being q (false | | |
| | acute versus | | discovery rate adjusted P | | |
| | recovery scores | | value) | | |

[symbol]

indicates data missing or illegible when filed

### REFERENCES

Anders, H.-J. and L. Schaefer (2014). “Beyond tissue injury-damage-associated molecular patterns, toll-like receptors, and inflammasomes also drive regeneration and fibrosis.” Journal of the American Society of Nephrology: JASN 25 (7): 1387-1400.
Anwar, M. A., M. Shah, J. Kim and S. Choi (2019). “Recent clinical trends in Toll-like receptor targeting therapeutics.” Medicinal research reviews 39 (3): 1053-1090.
Chauhan, A. S., P. V. Diwan, N. K. Jain and D. A. Tomalia (2009). “Unexpected in vivo anti-inflammatory activity observed for simple, surface functionalized poly (amidoamine) dendrimers.” Biomacromolecules 10 (5): 1195-1202.
Davidson, A., C. Berthier and M. Kretzler (2013). Chapter 18-Pathogenetic Mechanisms in Lupus Nephritis. Dubois' Lupus Erythematosus and Related Syndromes (Eighth Edition). D. J. Wallace and B. H. Hahn. Philadelphia, W.B. Saunders: 237-255.
Dawulieti, J., M. Sun, Y. Zhao, D. Shao, H. Yan, Y. H. Lao, H. Hu, L. Cui, X. Lv, F. Liu, C. W. Chi, Y. Zhang, M. Li, M. Zhang, H. Tian, X. Chen, K. W. Leong and L. Chen (2020). “Treatment of severe sepsis with nanoparticulate cell-free DNA scavengers.” Sci Adv 6 (22): eaay7148.
Dufour, J. H., M. Dziejman, M. T. Liu, J. H. Leung, T. E. Lane and A. D. Luster (2002). “IFN-gamma-inducible protein 10 (IP-10; CXCL10)-deficient mice reveal a role for IP-10 in effector T cell generation and trafficking.” J Immunol 168 (7): 3195-3204.
Eteshola, E. O. U., K. Landa, R. E. Rempel, I. A. Naqvi, E. S. Hwang, S. K. Nair and B. A. Sullenger (2021). “Breast cancer-derived DAMPs enhance cell invasion and metastasis, while nucleic acid scavengers mitigate these effects.” Mol Ther Nucleic Acids 26:1-10.
Fox, L. J., R. M. Richardson and W. H. Briscoe (2018). “PAMAM dendrimer—cell membrane interactions.” Advances in Colloid and Interface Science 257:1-18.
Gao, W., Y. Xiong, Q. Li and H. Yang (2017). “Inhibition of Toll-Like Receptor Signaling as a Promising Therapy for Inflammatory Diseases: A Journey from Molecular to Nano Therapeutics.” Frontiers in Physiology 8 (508).
Greish, K., G. Thiagarajan, H. Herd, R. Price, H. Bauer, D. Hubbard, A. Burckle, S. Sadekar, T. Yu, A. Anwar, A. Ray and H. Ghandehari (2012). “Size and surface charge significantly influence the toxicity of silica and dendritic nanoparticles.” Nanotoxicology 6 (7): 713-723.
Heiden, T. C. K., E. Dengler, W. J. Kao, W. Heideman and R. E. Peterson (2007). “Developmental toxicity of low generation PAMAM dendrimers in zebrafish.” Toxicology and applied pharmacology 225 (1): 70-79.
Holl, E. K., J. E. Bond, M. A. Selim, T. Ehanire, B. Sullenger and H. Levinson (2014). “The nucleic acid scavenger polyamidoamine third-generation dendrimer inhibits fibroblast activation and granulation tissue contraction.” Plast Reconstr Surg 134 (3): 420e-433e.
Holl, E. K., V. Frazier, K. Landa, D. Boczkowski, B. Sullenger and S. K. Nair (2021). “Controlling cancer-induced inflammation with a nucleic acid scavenger prevents lung metastasis in murine models of breast cancer.” Mol Ther 29 (5): 1772-1781.
Holl, E. K., K. L. Shumansky, L. B. Borst, A. D. Burnette, C. J. Sample, E. A. Ramsburg and B. A. Sullenger (2016). “Scavenging nucleic acid debris to combat autoimmunity and infectious disease.” Proceedings of the National Academy of Sciences 113 (35): 9728.
Jain, K., P. Kesharwani, U. Gupta and N. K. Jain (2010). “Dendrimer toxicity: Let's meet the challenge.” Int J Pharm 394 (1-2): 122-142.
Jain, S., G. A. Pitoc, E. K. Holl, Y. Zhang, L. Borst, K. W. Leong, J. Lee and B. A. Sullenger (2012). “Nucleic acid scavengers inhibit thrombosis without increasing bleeding.” Proceedings of the National Academy of Sciences 109 (32): 12938.
Kelly, L., L. B. Olson, R. E. Rempel, J. I. Everitt, D. Levine, S. K. Nair, M. E. Davis and B. A. Sullenger (2022). “beta-Cyclodextrin-containing polymer treatment of cutaneous lupus and influenza improves outcomes.” Mol Ther 30 (2): 845-854.
Labieniec-Watala, M. and C. Watala (2015). “PAMAM Dendrimers: Destined for Success or Doomed to Fail? Plain and Modified PAMAM Dendrimers in the Context of Biomedical Applications.” Journal of Pharmaceutical Sciences 104 (1): 2-14.
Lee, J., J. W. Sohn, Y. Zhang, K. W. Leong, D. Pisetsky and B. A. Sullenger (2011). “Nucleic acid-binding polymers as anti-inflammatory agents.” Proceedings of the National Academy of Sciences 108 (34): 14055.
Lesniak, W. G., M. K. Mishra, A. Jyoti, B. Balakrishnan, F. Zhang, E. Nance, R. Romero, S. Kannan and R. M. Kannan (2013). “Biodistribution of fluorescently labeled PAMAM dendrimers in neonatal rabbits: effect of neuroinflammation.” Molecular pharmaceutics 10 (12): 4560-4571.
Liang, H., B. Peng, C. Dong, L. Liu, J. Mao, S. Wei, X. Wang, H. Xu, J. Shen, H. Q. Mao, X. Gao, K. W. Leong and Y. Chen (2018). “Cationic nanoparticle as an inhibitor of cell-free DNA-induced inflammation.” Nat Commun 9 (1): 4291.
Liang, H., Y. Yan, J. Wu, X. Ge, L. Wei, L. Liu and Y. Chen (2020). “Topical nanoparticles interfering with the DNA-LL37 complex to alleviate psoriatic inflammation in mice and monkeys.” Sci Adv 6 (31): eabb5274.
Liu, F., S. Sheng, D. Shao, Y. Xiao, Y. Zhong, J. Zhou, C. H. Quek, Y. Wang, Z. Hu, H. Liu, Y. Li, H. Tian, K. W. Leong and X. Chen (2021). “A Cationic Metal-Organic Framework to Scavenge Cell-Free DNA for Severe Sepsis Management.” Nano Lett 21 (6): 2461-2469.
Malik, N., R. Wiwattanapatapee, R. Klopsch, K. Lorenz, H. Frey, J. W. Weener, E. W. Meijer, W. Paulus and R. Duncan (2000). “Dendrimers:: Relationship between structure and biocompatibility in vitro, and preliminary studies on the biodistribution of 125I-labelled polyamidoamine dendrimers in vivo.” Journal of Controlled Release 65 (1): 133-148.
Meyerholz, D. K., N. L. Tintle and A. P. Beck (2019). “Common Pitfalls in Analysis of Tissue Scores.” Vet Pathol 56 (1): 39-42.
Naqvi, I., N. Giroux, L. Olson, S. A. Morrison, T. Llanga, T. O. Akinade, Y. Zhu, Y. Zhong, S. Bose, S. Arvai, K. Abramson, L. Chen, L. Que, B. Kraft, X. Shen, J. Lee, K. W. Leong, S. K. Nair and B. Sullenger (2022). “DAMPs/PAMPs induce monocytic TLR activation and tolerance in COVID-19 patients; nucleic acid binding scavengers can counteract such TLR agonists.” Biomaterials 283:121393.
Naqvi, I., R. Gunaratne, J. E. McDade, A. Moreno, R. E. Rempel, D. C. Rouse, S. G. Herrera, D. S. Pisetsky, J. Lee, R. R. White and B. A. Sullenger (2018). “Polymer-Mediated Inhibition of Pro-invasive Nucleic Acid DAMPs and Microvesicles Limits Pancreatic Cancer Metastasis.” Mol Ther 26 (4): 1020-1031.
Naqvi, I., R. Gunaratne, J. E. McDade, A. Moreno, R. E. Rempel, D. C. Rouse, S. G. Herrera, D. S. Pisetsky, J. Lee, R. R. White and B. A. Sullenger (2018). “Polymer-Mediated Inhibition of Pro-invasive Nucleic Acid DAMPs and Microvesicles Limits Pancreatic Cancer Metastasis.” Molecular Therapy 26 (4): 1020-1031.
Peng, B., H. Liang, Y. Li, C. Dong, J. Shen, H. Q. Mao, K. W. Leong, Y. Chen and L. Liu (2019). “Tuned Cationic Dendronized Polymer: Molecular Scavenger for Rheumatoid Arthritis Treatment.” Angew Chem Int Ed Engl 58 (13): 4254-4258.
Pryor, J. B., B. J. Harper and S. L. Harper (2014). “Comparative toxicological assessment of PAMAM and thiophosphoryl dendrimers using embryonic zebrafish.” International journal of nanomedicine 9:1947-1956.
Roberts, J. C., M. K. Bhalgat and R. T. Zera (1996). “Preliminary biological evaluation of polyamidoamine (PAMAM) Starburst™ dendrimers.” Journal of Biomedical Materials Research 30 (1): 53-65.
Shi, C., J. Dawulieti, F. Shi, C. Yang, Q. Qin, T. Shi, L. Wang, H. Hu, M. Sun, L. Ren, F. Chen, Y. Zhao, F. Liu, M. Li, L. Mu, D. Liu, D. Shao, K. W. Leong and J. She (2022). “A nanoparticulate dual scavenger for targeted therapy of inflammatory bowel disease.” Sci Adv 8 (4): eabj2372.
Stearns, N. A., J. Lee, K. W. Leong, B. A. Sullenger and D. S. Pisetsky (2012). “The Inhibition of Anti-DNA Binding to DNA by Nucleic Acid Binding Polymers.” PLOS ONE 7 (7): e40862.
Tang, Y., Y. Han, L. Liu, W. Shen, H. Zhang, Y. Wang, X. Cui, Y. Wang, G. Liu and R. Qi (2015). “Protective effects and mechanisms of G5 PAMAM dendrimers against acute pancreatitis induced by caerulein in mice.” Biomacromolecules 16 (1): 174-182.
Tomalia, D. A., H. Baker, J. Dewald, M. Hall, G. Kallos, S. Martin, J. Roeck, J. Ryder and P. Smith (1985). “A New Class of Polymers: Starburst-Dendritic Macromolecules.” Polymer Journal 17 (1): 117-132.
Wu, Y.-w., W. Tang and J.-p. Zuo (2015). “Toll-like receptors: potential targets for lupus treatment.” Acta pharmacologica Sinica 36 (12): 1395-1407.
Yan, Y., H. Liang, X. Liu, L. Liu and Y. Chen (2021). “Topical cationic hairy particles targeting cell free DNA in dermis enhance treatment of psoriasis.” Biomaterials 276:121027.

1. A mixed-surface polyamidoamine (PAMAM) polymer comprising a plurality of surface groups, wherein the surface groups comprise cationic surface groups and neutral surface groups, wherein between about 30% and about 70% of the surface groups are neutral.

2. The PAMAM polymer of claim 1, wherein the surface groups consist of cationic surface groups and neutral surface groups, wherein between about 30% and about 70% of the surface groups are neutral.

3. The PAMAM polymer of claim 1, wherein the polymer is a G3 polymer or a G4 polymer.

4. The PAMAM polymer of claim 1, wherein about 50% of the surface groups are neutral.

5. The PAMAM polymer of claim 1, wherein the neutral surface groups comprise hydroxyl groups.

6.-11. (canceled)

12. A method of treating cancer in a subject comprising administering a therapeutically effective amount of the PAMAM polymer of claim 1 to the subject.

13. A method of treating cancer in a subject comprising contacting a bodily fluid from a subject with the PAMAM polymer of claim 1, wherein the contacting removes invasion-promoting or metastatic molecules or particles from the bodily fluid of the subject.

14. The method of claim 12, wherein the cancer is selected from the group consisting of pancreatic, ovarian, colon, liver, brain, breast, and lung cancer.

15. A method for reducing inflammatory mediators in a bodily fluid of a subject, the method comprising: contacting the bodily fluid with the PAMAM polymer of claim 1 wherein the contacting step reduces the concentration of the inflammatory mediators in the bodily fluid.

16. The method of claim 15, wherein the subject suffers from a condition associated with an abnormally high concentration of inflammatory mediators.

17. The method of claim 16, wherein the condition is an autoimmune disease, an infectious disease, or an inflammatory effect of radiation therapy or exposure to radiation.

18. The method of claim 13, wherein the contacting step is performed within the subject.

19. The method of claim 13, wherein the contacting step is performed outside the subject.

20. The method of claim 19, further comprising obtaining the bodily fluid from the subject before the contacting step.

21. The method of claim 20, further comprising returning the bodily fluid to the subject.

22. The method of claim 13, wherein the contacting step is performed during dialysis or in conjunction with extracorporeal membrane oxygenation.

23. The method of claim 13, wherein the bodily fluid is blood, lymph, plasma, serum, urine or cerebral spinal fluid.

24. The method of claim 12, wherein the PAMAM polymer is administered to the subject via a route selected from the group consisting of oral, topical, intranasal, intraperitoneal, parenteral, intravenous, intramuscular, subcutaneous, intrathecal, transcutaneous, nasopharyngeal, intratumoral, and transmucosal.

25. The method of claim 12, further comprising administering a therapeutic agent or radiation to the subject.

26.-51. (canceled)

52. A PAMAM composition comprising a plurality of nanofibers, the nanofibers comprising a plurality of mixed-surface polyamidoamine (PAMAM) polymers,
wherein the nanofibers are less than 2 μm in diameter, and
wherein each of the PAMAM polymers comprises cationic surface groups and neutral surface groups, wherein between about 30% and about 70% of the surface groups are neutral.

53.-64. (canceled)
